(19) United States
(12) Patent Application Publication
(10) Pub. No.: US 2025/0092418 A1
(43) Pub. Date: Mar. 20, 2025

(54) METHODS OF GENETICALLY ALTERING A PLANT NIN-GENE TO BE RESPONSIVE TO AUXIN

(71) Applicant: NIAB, Cambridge (GB)

(72) Inventors: Anindya KUNDU, Cambridge (GB); Richard J. HARRISON, Cambridge (GB)

(73) Assignee: NIAB, Cambridge (GB)

(21) Appl. No.: 18/816,990

(22) Filed: Aug. 27, 2024

Related U.S. Application Data
(60) Provisional application No. 63/579,266, filed on Aug. 28, 2023.

(51) Int. Cl. C12N 15/82; C07K 14/415

(52) U.S. Cl. CPC C12N 15/8294; C07K 14/415; C12N 15/8237

(57) ABSTRACT

Aspects of the present disclosure relate to genetically modified plants comprising NODULE INCEPTION (NIN) and NIN-LIKE PROTEIN (NLP) that have been genetically altered to be responsive to auxin so that the NIN or NLP protein can induce root nodulation upon appropriate signaling.

### CROSS REFERENCE TO RELATED APPLICATION

This application claims the benefit of U.S. Provisional Application No. 63/579,266, filed Aug. 28, 2023, which is hereby incorporated by reference in its entirety.

### REFERENCE TO AN ELECTRONIC SEQUENCE LISTING

The contents of the electronic sequence listing (794542002700seqlist.xml; Size: 1,069,688 bytes; and Date of Creation: Aug. 26, 2024) are herein incorporated by reference in their entirety.

### TECHNICAL FIELD

The present disclosure relates to genetically altered plants. In particular, the present disclosure relates to plants with NODULE INCEPTION (NIN) and NIN-LIKE PROTEIN (NLP) genes that have been genetically altered to be responsive to auxin so that the NIN or NLP protein can induce root nodulation upon appropriate signaling.

### BACKGROUND

Nodulating plant species, such as legumes, have evolved to form symbiotic relationships with nitrogen fixing bacteria. They form specialized organs called nodules to house these bacteria, which provide an optimal environment for the bacteria to fix nitrogen and provide it to the plant. In turn, the plant provides the bacteria with carbohydrates and other resources. The initial step of nodule formation is the recognition of the presence of symbiotic bacteria, for example by the detection of Nod factors produced by the bacteria. This recognition induces root hair curling, in which a tight curl is formed at the tip of the root hair. This tight curl establishes an infection chamber within which a bacterial colony can form that then extends an infection thread into the root. This infection thread triggers signaling within the root that ultimately results in the production of a new nodule.

Biological nitrogen fixation requires de novo organogenesis of root nodules where nitrogen fixing filamentous actinorhizal bacteria or rhizobia fix atmospheric nitrogen into ammonia. Genetic screens have identified genes involved in the nodule organogenesis process. Chief among these is the transcription factor NODULE INCEPTION (NIN), belonging to the NLP (NIN-Like Protein) superfamily, which has been shown to have a key role in nodule organogenesis across multiple plant species. While NLPs have recently been reported for their N-sensing capability and role in lateral root organogenesis, it is apparent that the nitrogen fixing plants neo-functionalized one of the NLPs (NIN) to act as a bridge between putative NLP functions and bacterial signal perception to initiate a nodule development program.

The precise mechanism of NIN involvement in organogenesis has remained elusive, because important components of NIN regulation and integration with nodule organogenesis processes were not known. More importantly, as NIN is the key player in nodule organogenesis, a successful nodule engineering approach needs to incorporate NIN. In order to successfully engineer nodulation, knowledge of NIN regulation will be required. There exists a clear need for identifying NIN regulatory regions that in combination with the NIN coding sequence will allow full regulation of NIN in engineering plants.

### BRIEF SUMMARY

In order to meet these needs, the present disclosure provides methods of introducing auxin responsive elements (AuxREs) into a regulatory region operably linked with the NIN coding sequence. The present disclosure further provides means of introducing cytokinin-responsive elements into plants operably linked with a NIN or NLP coding sequence that may be endogenous or heterologous.
1. A genetically altered plant or part thereof comprising one or more genetic alterations that increase activity of a NODULE INCEPTION (NIN) protein or a NIN-like protein (NLP protein) in response to auxin signaling as compared to a control plant without the one or more genetic alterations.
2. The genetically altered plant or part thereof of aspect 1, wherein the one or more genetic alterations comprise addition of one or more auxin response elements (AuxREs) operably linked to a nucleic acid encoding the NIN protein or the NLP protein.
3. The genetically altered plant or part thereof of aspect 2, wherein the one or more genetic alterations comprise addition of one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more AuxREs.
4. The genetically altered plant or part thereof of aspect 2 or aspect 3, wherein the one or more AuxREs comprise a TGTCTC motif or a TGTCTN motif.
5. The genetically altered plant or part thereof of any one of aspects 2-4, wherein the one or more AuxREs are added between a cyclops binding site and a transcriptional start site of the nucleic acid encoding the NIN protein or the NLP protein.
5b. The genetically altered plant or part thereof of any one of aspects 2-5, wherein the NIN protein or the NLP protein comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 649.
6. The genetically altered plant or part thereof of any one of aspects 2-5b, wherein the nucleic acid encodes a NIN protein, and wherein the NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588.
7. The genetically altered plant or part thereof of aspect 6, wherein the NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 578, SEQ ID NO: 579, SEQ ID NO: 553, and SEQ ID NO: 554.
8. The genetically altered plant or part thereof of any one of aspects 2-5b, wherein the nucleic acid encodes a NIN/NLP1 orthogroup protein, and wherein the NIN/NLP1 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588.
9. The genetically altered plant or part thereof of any one of aspects 2-5b, wherein the nucleic acid encodes a NLP2-3 orthogroup protein, and wherein the NLP2-3 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377.
10. The genetically altered plant or part thereof of any one of aspects 2-5b, wherein the nucleic acid encodes a NLP4 orthogroup protein, and wherein the NLP4 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524.
11. The genetically altered plant or part thereof of any one of aspects 2-5b, wherein the nucleic acid encodes a basal NIN/NLP orthogroup protein, and wherein the basal NIN/NLP orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550.
12. The genetically altered plant or part thereof of any one of aspects 2-11, wherein the one or more AuxREs are derived from a promoter of a nucleic acid encoding the NIN protein or the NLP protein in a legume or an actinorhizal plant species or wherein the one or more AuxREs are derived from a synthetic auxin responsive promoter, optionally wherein the synthetic auxin responsive promoter is pDR5.
13. The genetically altered plant or part thereof of any one of aspects 2-12, wherein the one or more AuxREs are heterologous or transfected.
14. The genetically altered plant or part thereof of any one of aspects 2-12, wherein the one or more Auxin REs are endogenous.
15. The genetically altered plant or part thereof of any one of aspects 2-14, wherein the nucleic acid encoding the NIN protein or the NLP protein is heterologous or transfected.
16. The genetically altered plant or part thereof of any one of aspects 2-14, wherein the nucleic acid encoding the NIN protein or the NLP protein is endogenous.
17. The genetically altered plant or part thereof of any one of aspects 1-16, wherein the genetically altered plant is a monocot, and wherein the genetically altered plant is selected from the group consisting of corn, rice, wheat, barley, sorghum, millet, oat, and rye.
18. The genetically altered plant or part thereof of any one of aspects 1-16, wherein the genetically altered plant is selected from the group consisting of apple, pear, plum, apricot, peach, almond, walnut, cherry, strawberry, raspberry, blackberry, red currant, black currant, melon, cucumber, pumpkin, squash, grape, hemp, hops, birch, beech, jujube, cassava, poplar, chestnut, citrus, potato, tomato, sweet potato, Datisca spp., Trema spp., and Jatropha spp.
19. The genetically altered plant or part thereof of aspect 17 or aspect 18, wherein the genetically altered plant is strawberry, barley, or Datisca spp. or the genetically altered plant is a non-nodulating plant and the increased activity of the NODULE INCEPTION (NIN) protein or the NIN-like protein (NLP protein) in response to auxin signaling induces formation of actinorhizal nodule-like structures.
20. A method of producing the genetically altered plant or part thereof of any one of aspects 1-19, comprising introducing a genetic alteration to the plant comprising a first nucleic acid sequence comprising one or more AuxREs.
21. The method of aspect 20, further comprising introducing a second nucleic acid sequence encoding the NIN protein or the NLP protein, wherein the first nucleic acid sequence and the second nucleic acid sequence are operably linked.
22. The method of aspect 20, wherein the first nucleic acid sequence is inserted into the genome of the plant so that the nucleic acid sequence is operably linked to an endogenous nucleic acid sequence encoding the NIN protein or the NLP protein.
23. The method of any one of aspects 20-22, wherein the one or more AuxREs comprise a TGTCTC motif or a TGTCTN motif.
24. The method of any one of aspects 20-23, wherein the first nucleic acid sequence comprises a synthetic auxin responsive promoter, optionally wherein the synthetic auxin responsive promoter is pDR5.
24b. The method of any one of aspects 21-24, wherein the NIN protein or the NLP protein comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 649.
25. The method of any one of aspects 21-24b, wherein the NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588; wherein the NIN/NLP1 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588; wherein the NLP2-3 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377; wherein the NLP4 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524; or wherein the basal NIN/NLP orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550.
26. A method of producing the genetically altered plant or part thereof of any one of aspects 1-19, comprising genetically modifying the plant or part thereof by transforming the plant or part thereof with one or more gene editing components that target an endogenous nuclear genome sequence between a cyclops binding site and a transcriptional start site of the nucleic acid encoding the NIN protein or the NLP protein and introduce one or more AuxREs.
27. The method of aspect 26, wherein the one or more gene editing components comprise a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.
28. The method of aspect 26 or aspect 27, wherein the one or more AuxREs comprise a TGTCTC motif or a TGTCTN motif.
28b. The method of any one of aspects 26-28, wherein the NIN protein or the NLP protein comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 649.
29. The method of any one of aspects 26-28b, wherein the NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588; wherein the NIN/NLP1 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588; wherein the NLP2-3 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377; wherein the NLP4 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524; or wherein the basal NIN/NLP orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550.
30. An expression vector, isolated DNA molecule, or recombinant nucleic acid comprising a nucleic acid sequence comprising one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more AuxREs from an endogenous plant promoter, or a synthetic promoter comprising one or more, two or more, three or more, four or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more AuxREs.
31. An expression vector, isolated DNA molecule, or recombinant nucleic acid comprising a nucleic acid sequence comprising one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more AuxREs operably linked to a second nucleic acid sequence encoding a NIN protein or a NLP protein.
32. The expression vector, isolated DNA molecule, or recombinant nucleic acid of aspect 30 or aspect 31, wherein the one or more AuxREs comprise a TGTCTC motif or a TGTCTN motif.
32b. The expression vector, isolated DNA molecule, or recombinant acid of aspect 31 or aspect 32, wherein the NIN protein or the NLP protein comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 649.
33. The expression vector, isolated DNA molecule, or recombinant nucleic acid of any one of aspects 31-32b, wherein the NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588; wherein the NIN/NLP1 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588; wherein the NLP2-3 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377; wherein the NLP4 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524; or wherein the basal NIN/NLP orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550.
34. A bacterial cell or an Agrobacterium cell comprising the expression vector, isolated DNA molecule, or recombinant nucleic acid of any one of aspects 30-33.
35. A genetically modified plant, plant part, plant cell, or seed comprising the expression vector, isolated DNA molecule, or recombinant nucleic acid of any one of aspects 30-33.
36. A composition or kit comprising the expression vector, isolated DNA molecule, or recombinant nucleic acid of any one of aspects 30-33, the bacterial cell or Agrobacterium cell of aspect 34, or the genetically modified plant, plant part, plant cell, or seed of aspect 35.
37. A non-regenerable part or cell of the genetically modified plant or part thereof of any one of aspects 1-19 or aspect 35.
38. A method of inducing symbiotic organogenesis, inducing symbiotic organogenesis in the absence of rhizobial bacteria recognized by the plant, and/or inducing infection of a symbiotic organ comprising introducing a genetic alteration via the expression vector, isolated DNA molecule, or recombinant nucleic acid of any one of aspects 30-33.
39. A genetically altered plant genome comprising (i) a first nucleic acid sequence encoding one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more auxin response elements (AuxREs) operably linked to a transfected or heterologous second nucleic acid encoding a NIN protein or an NLP protein, or (ii) genetic modifications adding one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more AuxREs to a promoter of an endogenous NIN protein or NLP protein.
40. A genetically altered plant genome of the plant of any one of aspects 1-19 or aspect 35.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIGS. 1A-1C show phylogenetically actinorhizal NINs as distant from the legume counterpart. FIG. 1A shows a maximum likelihood phylogenetic tree showing the NIN gene family within the NFN clade nodulators where the nodulators belonging to Fabales (legumes, bottom) branch away from the actinorhizal NINs (Rosales (top left), Cucurbitales (top center), and Fagales (top right)). All the NINs belonging to the Rosales clade cluster together (top left) indicating their unique identity among the actinorhizal NINs. FIG. 1B shows a box plot of relative expression of Datisca glomerata DgNIN (left) and DgNIN2 (right) in roots, early nodule, late nodule, and mature (senescent) nodule, normalized to DgActin. Values are the mean ΔCt values of 3 biological replicates. The X-axis indicates the tissue investigated; the Y-axis indicates relative expression. FIG. 1C shows a diagram of the NIN protein (top), comprising the N-terminal region (orange) followed by the DNA binding RWP-RK motif (green) and the protein-protein interacting PB1 domain (blue). A sequence alignment of a portion of the N-terminal region is shown below for legume NIN sequences (unshaded) and actinorhizal NIN sequences (shaded). The sequences are, from top to bottom beginning at “Glycine NIN extraction”, SEQ ID NO: 606, SEQ ID NO: 607, SEQ ID NO: 608, SEQ ID NO: 609, SEQ ID NO: 610, SEQ ID NO: 611, SEQ ID NO: 612, SEQ ID NO: 613, SEQ ID NO: 614, SEQ ID NO: 615, SEQ ID NO: 616, SEQ ID NO: 617, SEQ ID NO: 618, SEQ ID NO: 619, SEQ ID NO: 620, SEQ ID NO: 621, SEQ ID NO: 622, SEQ ID NO: 623, SEQ ID NO: 624, SEQ ID NO: 625, SEQ ID NO: 626, SEQ ID NO: 627, SEQ ID NO: 628, SEQ ID NO: 629, and SEQ ID NO: 630. SEQ ID NO: 631 is the Consensus sequence. The Serine whose phosphorylation status is known to be important for nuclear retention in Arabidopsis is shown in the yellow vertical box.

FIGS. 2A-2Q show nodulation-specific expression of actinorhizal NIN in Datisca glomerata. FIG. 2A shows expression of pDgNIN_5 kb:GUS in an uninoculated control plant in a stereo-fluorescence micrograph. The scale bar indicates 500 μm. FIG. 2B shows expression of pDgNIN_5 kb:GUS in early nodules of roots harvested 8 weeks after infection with clade 2 Frankia Dg1 in a stereo-fluorescence micrograph. The scale bar indicates 500 μm. FIG. 2C shows expression of pDgNIN_5 kb:GUS in a mature nodule of roots harvested 8 weeks after infection with clade 2 Frankia Dg1 in a stereo-fluorescence micrograph. The scale bar indicates 500 μm. FIG. 2D shows ultrastructure analysis of nodule sections stained with ruthenium red (pink coloration) of mature nodules with increased expression in the infection zone (iz) with vesicle formation. Meristem (m) and vascular bundle (vb, arrowhead) are indicated. The scale bar indicates 250 μm. FIG. 2E shows ultrastructure analysis of nodule sections stained with ruthenium red (pink coloration) of mature nodules with increased expression in the infection zone (iz) with vesicle formation. Uninfected cells (*) are also indicated. The scale bar indicates 250 μm. FIG. 2F shows expression of pDg2NIN_5 kb:GUS in an uninoculated control plant in a stereo-fluorescence micrograph. The scale bar indicates 500 μm. FIG. 2G shows expression of pDgNIN2_5 kb:GUS in early nodules of roots harvested 8 weeks after infection with clade 2 Frankia Dg1 in a stereo-fluorescence micrograph. The scale bar indicates 500 μm. FIG. 2H shows expression of pDgNIN2_5 kb:GUS in a mature nodule of roots harvested 8 weeks after infection with clade 2 Frankia Dg1 in a stereo-fluorescence micrograph. The scale bar indicates 500 μm. FIG. 2I shows ultrastructure analysis of nodule sections stained with ruthenium red (pink coloration) of mature nodules with increased expression in the infection zone (iz) with vesicle formation. Vascular bundle (vb, arrowhead) is indicated. The scale bar indicates 250 μm.

FIG. 2J shows ultrastructure analysis of nodule sections stained with ruthenium red (pink coloration) of mature nodules with increased expression in the infection zone (iz) with vesicle formation. The scale bar indicates 250 μm. FIG. 2K shows expression of pDryasNIN_5 kb:GUS in an uninoculated control plant in a stereo-fluorescence micrograph (top), with a micrograph of the transformation marker mCherry (bottom). The scale bar indicates 500 μm. FIG. 2L shows expression of pDryasNIN_5 kb:GUS in early nodule of roots harvested 8 weeks after infection with clade 2 Frankia Dg1 in a stereo-fluorescence micrograph. The scale bar indicates 500 μm.

FIG. 2M shows expression of pDryasNIN_5 kb:GUS in mature nodule of roots harvested 8 weeks after infection with clade 2 Frankia Dg1 in a stereo-fluorescence micrograph. The scale bar indicates 500 μm. FIG. 2N shows ultrastructure analysis of nodule sections from roots expressing pDryasNIN:GUS stained with ruthenium red (pink coloration) of matured nodules with expression in infected cells (pink). Vascular bundle (vb, arrowhead) and infection zone (iz) are indicated. The scale bar indicates 250 μm. FIG. 2O shows ultrastructure analysis of nodule sections from roots expressing pDryasNIN:GUS stained with ruthenium red (pink coloration) of matured nodules with expression in infected cells (pink). Vascular bundle (vb, arrowhead) and infection zone (iz) are indicated. The scale bar indicates 250 μm. FIG. 2P shows a stereomicrograph of GUS stained D. glomerata hairy-root transformed roots transformed with pDatisca_glomerata_NIN_5k:GUS. The top panel (P) shows bright field; the bottom panel (P′) shows mCherry fluorescence (transformation marker). The scale bar indicates 500 μm. FIG. 2Q shows semithin sections of GUS stained D. glomerata hairy-root transformed roots transformed with pDatisca_glomerata_NIN_5k:GUS counterstained with ruthenium red. The scale bar indicates 250 μm. Meristem is indicated by ‘m’, cortex is indicated by ‘c’, and vascular bundle is indicated by ‘vb’. FIG. 2R shows a stereomicrograph of GUS stained D. glomerata hairy-root transformed roots transformed with pDryas_drummondii_NIN_5k:GUS. The top panel (R) shows bright field; the bottom panel (R′) shows mCherry fluorescence (transformation marker). The scale bar indicates 500 μm. FIG. 2S shows semithin sections of GUS stained D. glomerata hairy-root transformed roots transformed with pDryas_drummondii_NIN_5k:GUS counterstained with ruthenium red. The scale bar indicates 250 μm. Meristem is indicated by ‘m’, cortex is indicated by ‘c’, and vascular bundle is indicated by ‘vb’.

FIGS. 3A-3I show that expression of actinorhizal NIN can match pMedicagoNIN promoter expression during nodulation in M. truncatula. FIG. 3A shows a stereo-fluorescence micrograph of A17 M. truncatula hairy-roots showing expression of pMtNINsyn:GUS at 2 weeks after infection (top), and a merged image of S. meliloti 2011 eGFP and transformation marker mCherry fluorescence (bottom, A′). The scale bar indicates 250 μm. FIG. 3B shows a stereo-fluorescence micrograph of A17 M. truncatula hairy-roots showing expression of pMtNINsyn:GUS at 4 weeks after infection (top), and a merged image of S. meliloti 2011 eGFP and the transformation marker mCherry fluorescence (right, B′). The scale bar indicates 500 μm.

FIG. 3C shows a section at lower (left, C) and higher magnification (right, C′) of A17 M. truncatula hairy-roots showing expression of pMtNINsyn:GUS at 4 weeks after infection, counterstained with Ruthenium red. The scale bar indicates 250 μm. FIG. 3D shows a stereo-fluorescence micrograph of A17 M. truncatula hairy-roots showing expression of pDatiscaNIN_5 kb:GUS at 2 weeks after infection (top), and a merged image of S. meliloti 2011 eGFP and transformation marker mCherry fluorescence (bottom, D′). The scale bar indicates 250 μm. FIG. 3E shows a stereo-fluorescence micrograph of A17 M. truncatula hairy-roots showing expression of pDatiscaNIN_5 kb:GUS at 4 weeks after infection (left), and a merged image of S. meliloti 2011 eGFP and the transformation marker mCherry fluorescence (right, E′). The scale bar indicates 500 μm. FIG. 3F shows a section at lower (left, F) and higher magnification (right, F′) of A17 M. truncatula hairy-roots showing expression of pDatiscaNIN_5 kb:GUS at 4 weeks after infection, counterstained with Ruthenium red. The scale bar indicates 250 μm. FIG. 3G shows a stereo-fluorescence micrograph of A17 M. truncatula hairy-roots showing expression of pDryasNIN_5 kb:GUS at 2 weeks after infection (top), and a merged image of S. meliloti 2011 eGFP and transformation marker mCherry fluorescence (bottom, G′). The scale bar indicates 250 μm. FIG. 3H shows a stereo-fluorescence micrograph of A17 M. truncatula hairy-roots showing expression of pDatiscaNIN_5 kb:GUS at 4 weeks after infection (top), and a merged image of S. meliloti 2011 eGFP and the transformation marker mCherry fluorescence (right, H′). The scale bar indicates 500 μm. FIG. 3I shows a section at lower (left, I) and higher magnification (right, I′) of A17 M. truncatula hairy-roots showing expression of pDryasNIN_5 kb:GUS at 4 weeks after infection, counterstained with Ruthenium red. The scale bar indicates 250 μm.

FIGS. 4A-4K show that exogenous treatment with a chitinous signaling molecule induces pDatiscaNIN and pDatiscaNIN2 expression. FIG. 4A shows a stereo micrograph of control untreated roots of D. glomerata hairy-root transformed plants carrying a pDgNIN-GUS construct. The bottom panel (A′) shows the boxed region from the top panel (A) at a higher magnification. The scale bar indicates 500 μm. FIG. 4B shows a stereo micrograph of roots of D. glomerata hairy-root transformed plants carrying a pDgNIN-GUS construct, 24 hours after treatment with 10⁻⁷M nonsulphated-LCO (NS-LCO). The bottom panel (B′) shows the boxed region from the top panel (B) at a higher magnification. The scale bar indicates 500 μm. FIG. 4C shows a stereo micrograph of roots of D. glomerata hairy-root transformed plants carrying a pDgNIN-GUS construct, 24 hours after treatment with 10⁻⁷M sulphated-LCO (S-LCO). The bottom panel (C′) shows the boxed region from the top panel (C) at a higher magnification. The scale bar indicates 500 μm. FIG. 4D shows a stereo micrograph of roots of D. glomerata hairy-root transformed plants carrying a pDgNIN-GUS construct, 24 hours after treatment with 10⁻⁷M CO4. The bottom panel (D′) shows the boxed region from the top panel (D) at a higher magnification. The scale bar indicates 500 μm. FIG. 4E shows a stereo micrograph of roots of D. glomerata hairy-root transformed plants carrying a pDgNIN-GUS construct, 24 hours after treatment with 10⁻⁷M CO8. The bottom panel (E′) shows the boxed region from the top panel (E) at a higher magnification. The scale bar indicates 500 μm. FIG. 4F shows a stereo micrograph of control untreated roots of D. glomerata hairy-root transformed plants carrying a pDgNIN2-GUS construct. The bottom panel (F′) shows the boxed region from the top panel (F) at a higher magnification. The scale bar indicates 500 μm. FIG. 4G shows a stereo micrograph of roots of D. glomerata hairy-root transformed plants carrying a pDgNIN2-GUS construct, 24 hours after treatment with 10⁻⁷M nonsulphated-LCO (NS-LCO). The bottom panel (G′) shows the boxed region from the top panel (G) at a higher magnification. The scale bar indicates 500 μm. FIG. 4H shows a stereo micrograph of roots of D. glomerata hairy-root transformed plants carrying a pDgNIN2-GUS construct, 24 hours after treatment with 10⁻⁷M sulphated-LCO (S-LCO). The bottom panel (H′) shows the boxed region from the top panel (H) at a higher magnification. The scale bar indicates 500 μm. FIG. 4I shows a stereo micrograph of roots of D. glomerata hairy-root transformed plants carrying a pDgNIN2-GUS construct, 24 hours after treatment with 10⁻⁷M CO4. The bottom panel (I′) shows the boxed region from the top panel (I) at a higher magnification. The scale bar indicates 500 μm. FIG. 4J shows a stereo micrograph of roots of D. glomerata hairy-root transformed plants carrying a pDgNIN2-GUS construct, 24 hours after treatment with 10⁻⁷M CO8. The bottom panel (J′) shows the boxed region from the top panel (J) at a higher magnification. The scale bar indicates 500 μm. FIG. 4K shows ultrastructure analysis of roots expressing a pDgNIN1-GUS construct (top) or a pDgNIN2-GUS construct (bottom), stained with ruthenium red (pink coloration), 24 hours after treatment with 10⁻⁷M S-LCO. The panels on the right show a higher magnification of the panels on the left. The scale bar indicates 500 μm.

FIGS. 5A-5G show that downregulation of DgNIN abolishes nodule development in D. glomerata. FIG. 5A shows boxplots representing the nodule number per root in transformed (green, left) and non-transformed (right, purple) hairy-roots of the transgenic plants expressing DgNIN-RNAi (second and fourth columns) as compared to empty vector transformed plants (first and third columns). The box indicates median (thick line) and second through third quartiles. Wilcoxon signed-rank test was used to determine statistical significance, shown by p-value and asterisks over brackets, where * indicates p<0.01; ** indicates p<0.002; *** indicates p<0.0003; indicates p<0.00006. n represents the number of transformed roots scored. The X-axis indicates the treatment; the Y-axis indicates the number of nodules per root. FIG. 5B shows a stereo-fluorescent micrograph (top, B) of empty vector transformed roots and nodules, and a corresponding image (bottom, B′) showing the eGFP transformation marker. The perforated box indicates the region shown at higher magnification in FIG. 5C. The scale bar indicates 500 μm.

FIG. 5C shows a stereo-fluorescent micrograph (top, C) of empty vector transformed roots and nodules, and a corresponding image (bottom, C′) showing the eGFP transformation marker. The scale bar indicates 500 μm. FIG. 5D shows a stereo-fluorescent micrograph (top, D) of DgNIN-RNAi transformed roots with no nodule formation, and a corresponding image (bottom, D′) showing the eGFP transformation marker. The scale bar indicates 500 μm. FIG. 5E shows a stereo-fluorescent micrograph (top, E) of DgNIN-RNAi transformed roots with cluster root formation, and a corresponding image (bottom, E′) showing the eGFP transformation marker. The perforated box indicates the region shown at higher magnification in FIG. 5F. The perforated line demarcates transformed roots from non-transformed roots. The scale bar indicates 500 μm. FIG. 5F shows a stereo-fluorescent micrograph (top, F) of DgNIN-RNAi transformed roots with cluster root formation, and a corresponding image (bottom, F′) showing the eGFP transformation marker. The perforated line demarcates transformed roots from non-transformed roots. The scale bar indicates 500 μm. FIG. 5G shows boxplots representing the relative expression of DgNIN (left) and DgNIN2 (right) in empty vector transformed roots (pK7_root), empty vector transformed nodule (pK7_nod), and four lines of roots transformed with DgNIN-RNAi (DgNIN_RNAi-2, DgNIN_RNAi-3, DgNIN_RNAi-5, DgNIN_RNAi-7) normalized to DgActin. Values are the mean ΔCt values of 3 biological replicates where T-test determined statistical significance, where * indicates p<0.01; ** indicates p<0.002; *** indicates p<0.0003; **** indicates p<0.00006. The X-axis indicates the construct; the Y-axis indicates relative expression of DgNIN or DgNIN normalized to DgActin.

FIGS. 6A-6C show spatial difference in expression of pLJUBI vs pDR5. FIG. 6A shows a stereo micrograph of control, uninoculated D. glomerata hairy roots transformed with pLJUBI:GUS. The panel on the right shows a region of the panel on the left at a higher magnification. The scale bar indicates 500 μm. FIG. 6B shows a stereo micrograph of control, uninoculated D. glomerata hairy roots transformed with pDR5:GUS. The panel on the right shows a region of the panel on the left at a higher magnification. The scale bar indicates 500 μm.

FIG. 6C shows a stereo micrograph of D. glomerata hairy roots transformed with pDR5:GUS eight weeks after infection with clade 2 Frankia Dg1. The panel on the right shows a region of the panel on the left at a higher magnification. The scale bar indicates 500 μm.

FIGS. 7A-7O show that ectopic expression of NIN disrupts lateral root development and nodulation homeostasis. FIG. 7A shows a stereo-fluorescence micrograph of empty vector transformed roots under control/non-inoculated conditions. The scale bar indicates 500 μm. FIG. 7B shows a stereo-fluorescence micrograph of empty vector transformed roots eight weeks after infection with clade 2 Frankia Dg1. The scale bar indicates 500 μm. FIG. 7C shows a stereo-fluorescence micrograph of a half-section of a nodule with an infection zone (iz) from empty vector transformed roots eight weeks after infection with clade 2 Frankia Dg1. The scale bar indicates 500 μm. FIG. 7D shows a stereo-fluorescence micrograph of pLJUBI: DatiscaNIN transformed roots under control/non-inoculated conditions (top, D), and a corresponding micrograph of the transformation marker mCherry (bottom, D′). The scale bar indicates 500 μm.

FIG. 7E shows a stereo-fluorescence micrograph of pLJUBI: DatiscaNIN transformed roots eight weeks after infection with clade 2 Frankia Dg1 (top, E), and a corresponding micrograph of the transformation marker mCherry (bottom, E′). The scale bar indicates 500 μm. FIG. 7F shows a stereo-fluorescence micrograph of a half-section of a proliferating structure from pLJUBI: DatiscaNIN transformed roots eight weeks after infection with clade 2 Frankia Dg1 (top, F), and a corresponding micrograph of the transformation marker mCherry (bottom, F′). The scale bar indicates 500 μm. FIG. 7G shows a stereo-fluorescence micrograph of pLJUBI: DatiscaNIN2 transformed roots under control/non-inoculated conditions (top, G), and a corresponding micrograph of the transformation marker mCherry (bottom, G′). FIG. 7H shows a stereo-micrograph of pLJUBI: DatiscaNIN2 transformed roots eight weeks after infection with clade 2 Frankia Dg1 (top, H), and a corresponding micrograph of the transformation marker mCherry (bottom, H′). The scale bar indicates 500 μm. FIG. 7I shows a stereo-fluorescence micrograph of a half-section of a proliferating section from pLJUBI: DatiscaNIN2 transformed roots eight weeks after infection with clade 2 Frankia Dg1 (top, I), and a corresponding micrograph of the transformation marker mCherry (bottom, I′). FIG. 7J shows a stereo-fluorescence micrograph of pLjUBI:DryasNIN transformed roots under control/non-inoculated conditions (top, J), and a corresponding micrograph of the transformation marker mCherry (bottom, J′). The scale bar indicates 500 μm. FIG. 7K shows a stereo-fluorescence micrograph of pLJUBI:DryasNIN transformed roots eight weeks after infection with clade 2 Frankia Dg1 (top, K), and a corresponding micrograph of the transformation marker mCherry (bottom, K′). The scale bar indicates 500 μm. FIG. 7L shows a stereo-fluorescence micrograph of a half-section of a proliferating structure from pLJUBI:DryasNIN transformed roots eight weeks after infection with clade 2 Frankia Dg1 (top, L), and a corresponding micrograph of the transformation marker mCherry (bottom, L′). The scale bar indicates 500 μm. FIG. 7M shows boxplots representing the number of the indicated structures per root of the transgenic plants under control/non-inoculated conditions for each indicated construct. The box indicates median (thick line) and second through third quartiles. From left to right, the graphs indicate the number of cluster roots per root, the number of nodules per root, and the number of pseudonodules per root. Within each graph, from left to right the box plots indicate the following constructs: empty vector, LjUBI: DatiscaNIN, LJUBI: DatiscaNIN2, and LJUBI:DryasNIN. The number at the bottom indicates the number of roots scored. Student's t-test followed by Bonferroni correction indicates statistically significant differences between the observations where **=p<0.001 and *=p<0.03. The values below each box represent the total number of transformed roots scored. FIG. 7N shows boxplots representing the number of the indicated structures per root of the transgenic plants eight weeks after infection with clade 2 Frankia Dg1 for each indicated construct. The box indicates median (thick line) and second through third quartiles. From left to right, the graphs indicate the number of cluster roots per root, the number of nodules per root, and the number of pseudonodules per root. Within each graph, from left to right the box plots indicate the following constructs: empty vector, LjUBI: DatiscaNIN, LJUBI: DatiscaNIN2, and LJUBI:DryasNIN. The number at the bottom indicates the number of roots scored. Student's t-test followed by Bonferroni correction indicates statistically significant differences between the observations where **=p<0.001 and *=p<0.03. The values below each box represent the total number of transformed roots scored. FIG. 7O shows Datisca glomerata hairy roots transformed with pDatiscaNIN:GUS. The top panel (O) shows a bright field image of the roots; the bottom panel (O′) shows an ImageQuant micrograph of mCherry fluorescence (pink, transformation marker) and non-transformed parts (blue mask). The scale bar indicates 2.4 cm. FIG. 7P shows Datisca glomerata hairy roots transformed with pLjUBI:DatiscaNIN. The top panel (P) shows a bright field image of the roots; the bottom panel (P′) shows an ImageQuant micrograph of mCherry fluorescence (pink, transformation marker) and non-transformed parts (blue mask). The scale bar indicates 2.4 cm. FIG. 7Q shows Datisca glomerata hairy roots transformed with pLjUBI:DatiscaNIN2. The top panel (Q) shows a bright field image of the roots; the bottom panel (Q′) shows an ImageQuant micrograph of mCherry fluorescence (pink, transformation marker) and non-transformed parts (blue mask). The scale bar indicates 2.4 cm.

FIGS. 8A-8P show that both Datisca and Dryas NIN can rescue nodulation in the M. truncatula nin1 mutant. FIG. 8A shows a cartoon illustrating categories of infection thread (IT) phenotypes—curl (left), where the bacteria is trapped in the shepherd's hook; extending (middle), where the infection thread extended to the epidermis but failed to progress further; and patch (right), where the infection thread is misdirected in the epidermal/upper cortical layer. FIG. 8B shows a stereo-fluorescence micrograph of nin1 mutant four weeks after infection with S. meliloti 2011 pNifH-eGFP transformed with empty vector control. The scale bar indicates 250 μm. FIG. 8C shows a stereo-fluorescence micrograph of nin mutant four weeks after infection with S. meliloti 2011 pNifH-eGFP transformed with pMtNINsyn:MtNIN, with infection thread curl phenotype visible as a merge between bright field and eGFP (purple). The scale bar indicates 250 μm. FIG. 8D shows a stereo-fluorescence micrograph of nin mutant four weeks after infection with S. meliloti 2011 pNifH-eGFP transformed with pMtNINsyn:MtNIN, with infection thread extending phenotype visible as a merge between bright field and eGFP (purple). The scale bar indicates 250 μm. FIG. 8E shows a stereo-fluorescence micrograph of nin mutant four weeks after infection with S. meliloti 2011 pNifH-eGFP transformed with pMtNINsyn:MtNIN, with infection thread patch phenotype visible as a merge between bright field and eGFP (purple). The scale bar indicates 250 μm. FIG. 8F shows a stereo-fluorescence micrograph of nin mutant four weeks after infection with S. meliloti 2011 pNifH-eGFP transformed with pMtNINsyn:MtNIN, with nodule phenotype visible as a merge between bright field and eGFP (purple). The scale bar indicates 250 μm. FIG. 8G shows a stereo-fluorescence micrograph of nin mutant four weeks after infection with S. meliloti 2011 pNifH-eGFP transformed with pMtNINsyn:DatiscaNIN, with infection thread curl phenotype visible as a merge between bright field and eGFP (purple). The scale bar indicates 250 μm. FIG. 8H shows a stereo-fluorescence micrograph of nin mutant four weeks after infection with S. meliloti 2011 pNifH-eGFP transformed with pMtNINsyn:DatiscaNIN, with infection thread extending phenotype visible as a merge between bright field and eGFP (purple). The scale bar indicates 250 μm. FIG. 8I shows a stereo-fluorescence micrograph of nin mutant four weeks after infection with S. meliloti 2011 pNifH-eGFP transformed with pMtNINsyn:DatiscaNIN, with infection thread patch phenotype visible as a merge between bright field and eGFP (purple). The scale bar indicates 250 μm. FIG. 8J shows a stereo-fluorescence micrograph of nin mutant four weeks after infection with S. meliloti 2011 pNifH-eGFP transformed with pMtNINsyn:DatiscaNIN, with nodule phenotype visible as a merge between bright field and eGFP (purple). The scale bar indicates 250 μm. FIG. 8K shows a stereo-fluorescence micrograph of nin mutant four weeks after infection with S. meliloti 2011 pNifH-eGFP transformed with pMtNINsyn:DryasNIN, with infection thread curl phenotype visible as a merge between bright field and eGFP (purple). The scale bar indicates 250 μm. FIG. 8L shows a stereo-fluorescence micrograph of nin mutant four weeks after infection with S. meliloti 2011 pNifH-eGFP transformed with pMtNINsyn:DryasNIN, with infection thread extending phenotype visible as a merge between bright field and eGFP (purple). The scale bar indicates 250 μm. FIG. 8M shows a stereo-fluorescence micrograph of nin mutant four weeks after infection with S. meliloti 2011 pNifH-eGFP transformed with pMtNINsyn:DryasNIN, with infection thread patch phenotype visible as a merge between bright field and eGFP (purple). The scale bar indicates 250 μm. FIG. 8N shows a stereo-fluorescence micrograph of nin mutant four weeks after infection with S. meliloti 2011 pNifH-eGFP transformed with pMtNINsyn:DryasNIN, with nodule phenotype visible as a merge between bright field and eGFP (purple). The scale bar indicates 250 μm. FIG. 8O shows boxplots representing the infection thread number with the given phenotype per root, for curl (left), extended (middle), and patch (right) phenotypes. Boxes show median (thick line) and second through third quartiles. Wilcoxon signed-rank test was used to determine statistical significance, shown by p-value and asterisks over brackets, where * indicates p<0.01; ** indicates p<0.002; *** indicates p<0.0003; **** indicates p<0.00006. n represents the number of transformed roots scored. The X-axis indicates the transformed construct; the Y-axis indicates the number of infection threads with the given phenotype per root. FIG. 8P shows boxplots representing the nodule number per root. Boxes show median (thick line) and second through third quartiles, with vertical lines to minimum and maximum. Wilcoxon signed-rank test was used to determine statistical significance, shown by p-value and asterisks over brackets, where * indicates p<0.01; ** indicates p<0.002; *** indicates p<0.0003; **** indicates p<0.00006. n represents the number of transformed roots scored. The X-axis indicates the background and transformed construct; the Y-axis indicates the number of nodules per root.

FIGS. 9A-9F show the presence of auxin responsive elements (AuxREs) in the promoter of NIN. FIG. 9A shows boxplots representing the relative expression of DgNIN (left) and DgNIN2 (right) in D. glomerata wild-type plant roots 10 days post treatment (DPT) with mock (control) versus 100⁻⁶M NAA, normalized to DgActin. Values are the mean ΔCt values of 3 biological replicates. Boxes show median (thick line) and second through third quartiles. T-test was used to determine statistical significance, shown by p-value and asterisks over brackets, where * indicates p<0.01; ** indicates p<0.002; *** indicates p<0.0003; **** indicates p<0.00006. The X-axis indicates the treatment; the Y-axis indicates the relative expression. FIG. 9B shows bar graphs representing the relative expression of MtNIN in M. truncatula roots treated with, from left to right, mock treatment, 0.1 μM IBA (Indole-3-butyric acid), 1 μM IBA, or 10 μM IBA for 16 hours (left panel) and 2 days (right panel). Significance and p-values are listed about the bars. FIG. 9C shows bar graphs representing the relative expression of MtNIN in M. truncatula roots treated with, from left to right, mock treatment, 0.1 μM 2,4D (2,4-Dichlorophenoxyacetic acid), 1 μM 2,4D, or 10 μM 2,4D for 16 hours (left panel) and 2 days (right panel). Significance and p-values are listed about the bars. FIG. 9D shows bar graphs representing the relative expression of MtNIN in M. truncatula roots treated with, from left to right, mock treatment, 0.1 μM NAA (1-Napthaleneacetic acid), 1 μM NAA, or 10 μM NAA for 16 hours (left panel) and 2 days (right panel). Significance and p-values are listed about the bars. Student's t-test followed by Bonferroni correction indicates statistically significant differences between the observations where ***=p<0.0003, **=p<0.003, and *=p<0.01. FIG. 9E shows a ridge plot of the region between the cyclops bindings site (PACE)(grey bar at 0) and the transcriptional start site ATG, showing the position of the two putative auxin response elements (AuxRE) in the promoter of the legume (top) and actinorhizal (bottom) NIN. The X-axis shows number of nucleotides from PACE. Within each plot, the TGTCTN (orange) motif is represented higher up than the TGTCTC motif (blue). FIG. 9F shows family-based plots for the AuxRE, indicating the presence of putative AuxREs in the following families from top to bottom: Vicioid clade, Loteae, Phaseoleae, Dalbergieae, Genisteae, Casuarinaceae, Betulaceae, Cannabaceae, Cucurbitaceae, Rhamnaceae, and Rosaceae. Each bar in the plot represents one motif, which are normalized in height depending upon the proximity of the next motif. The TGTCTN motif is shown in orange, and the TGTCTC motif is shown in blue. The sequences used for this analysis were SEQ ID NO: 589, SEQ ID NO: 590, SEQ ID NO: 591, SEQ ID NO: 592, SEQ ID NO: 593, SEQ ID NO: 594, SEQ ID NO: 595, SEQ ID NO: 596, SEQ ID NO: 597, SEQ ID NO: 598, SEQ ID NO: 599, SEQ ID NO: 600, SEQ ID NO: 601, SEQ ID NO: 602, SEQ ID NO: 603, SEQ ID NO: 604, and SEQ ID NO: 605.

FIGS. 10A-10O show that synthetic auxin responsive promoter-driven NIN can induce spontaneous nodulation in D. glomerata. FIG. 10A shows a cartoon representation of the design for the experimental constructs pDR5:DatiscaNIN (top) and pDR5:DryasNIN (bottom). FIG. 10B shows a stereo-fluorescence micrograph of roots transformed with an empty vector under control/non-inoculated conditions. FIG. 10C shows a stereo-fluorescence micrograph of normal nodules in roots transformed with an empty vector eight weeks after infection with clade 2 Frankia Dg1. FIG. 10D shows a stereo-fluorescence micrograph of spontaneous nodule-like structures forming in roots transformed with pDR5:DatiscaNIN under control/non-inoculated conditions (top, D), and a corresponding micrograph of the transformation marker mCherry (bottom, D′). The scale bar indicates 500 μm. FIG. 10E shows a stereo-fluorescence micrograph of merged structures forming in roots transformed with pDR5:DatiscaNIN under control/non-inoculated conditions (top, E), and a corresponding micrograph of the transformation marker mCherry (bottom, E′). The scale bar indicates 500 μm. FIG. 10F shows a stereo-fluorescence micrograph of spontaneous nodule-like structures forming in roots transformed with pDR5:DatiscaNIN eight weeks after inoculation with clade 2 Frankia Dg1 (top, F), and a corresponding micrograph of the transformation marker mCherry (bottom, F′). The scale bar indicates 500 μm. FIG. 10G shows a stereo-fluorescence micrograph of nodules in roots transformed with pDR5:DatiscaNIN eight weeks after inoculation with clade 2 Frankia Dg1 (top, G), and a corresponding micrograph of the transformation marker mCherry (bottom, G′). The scale bar indicates 500 μm. FIG. 10H shows boxplots representing the number of bumps per root under control (left; green) and inoculated (right; purple) conditions for roots transformed with Empty vector (first and fourth columns), pDR5:DatiscaNIN (second and fifth columns), and pDR5:DryasNIN (third and sixth columns). The box indicates median (thick line) and second through third quartiles. Wilcoxon signed-rank test was used to determine statistical significance, shown by p-value and asterisks over brackets, where * indicates p<0.01; ** indicates p<0.002; *** indicates p<0.0003; **** indicates p<0.00006. n represents the number of transformed roots scored. The X-axis indicates the treatment; the Y-axis indicates the number of bumps per root. FIG. 10I shows boxplots representing the number of merged structures per root under control (left; green) and inoculated (right; purple) conditions for roots transformed with Empty vector (first and fourth columns), pDR5:DatiscaNIN (second and fifth columns), and pDR5:DryasNIN (third and sixth columns). The box indicates median (thick line) and second through third quartiles, with vertical lines to minimum and maximum. Wilcoxon signed-rank test was used to determine statistical significance, shown by p-value and asterisks over brackets, where * indicates p<0.01; ** indicates p<0.002; *** indicates p<0.0003; **** indicates p<0.00006. n represents the number of transformed roots scored. The X-axis indicates the treatment; the Y-axis indicates the number of merged structures per root. FIG. 10J shows a stereo-fluorescence micrograph of spontaneous nodule-like structures forming in roots transformed with pDR5:DryasNIN under control/non-inoculated conditions (top, J), and a corresponding micrograph of the transformation marker mCherry (bottom, J′). The scale bar indicates 500 μm. FIG. 10K shows a stereo-fluorescence micrograph of merged structures forming in roots transformed with pDR5:DryasNIN under control/non-inoculated conditions (top, K), and a corresponding micrograph of the transformation marker mCherry (bottom, K′). The scale bar indicates 500 μm. FIG. 10L shows a stereo-fluorescence micrograph of spontaneous nodule-like structures forming in roots transformed with pDR5:DryasNIN eight weeks after inoculation with clade 2 Frankia Dg1 (top, L), and a corresponding micrograph of the transformation marker mCherry (bottom, L′). The scale bar indicates 500 μm. FIG. 10M shows a stereo-fluorescence micrograph of nodules in roots transformed with pDR5:DryasNIN eight weeks after inoculation with clade 2 Frankia Dg1 (top, M), and a corresponding micrograph of the transformation marker mCherry (bottom, M′). The scale bar indicates 500 μm. FIG. 10N shows boxplots representing the number of nodules per root under control (left; green) and inoculated (right; purple) conditions for roots transformed with Empty vector (first and fourth columns), pDR5:DatiscaNIN (second and fifth columns), and pDR5:DryasNIN (third and sixth columns). The box indicates median (thick line) and second through third quartiles. Wilcoxon signed-rank test was used to determine statistical significance, shown by p-value and asterisks over brackets, where * indicates p<0.01; ** indicates p<0.002; *** indicates p<0.0003; **** indicates p<0.00006. n represents the number of transformed roots scored. The X-axis indicates the treatment; the Y-axis indicates the number of nodules per root. FIG. 10O shows ultrastructure analysis of nodule like-structure induced by synthetic auxin responsive promoter driving NIN. Panels 1, 2, 4, 5, 7, and 8 show stereomicrographs of hairy-root transformed roots showing semi-thin sections stained with toluidine blue for: Panels 1-2: clade 2 Frankia Dg1 induced nodules in empty vector control (8 weeks post infection); Panels 4-5: nodule like-structures in roots transformed with pDR5:DatiscaNIN; Panels 7-8: nodule=like structures in roots transformed with pDR5:DryasNIN. Micrographs of nodules are shown in panel 3; micrographs of nodule-like structures are shown in panels 6 and 9. The scale bars indicate: Panels 1, 4, 7: 500 m; Panels 2, 3, 5, 6, 8, and 9: 250 μm.

FIGS. 11A-11J show pDR5:GUS promoter activity in D. glomerata versus M. truncatula A17 and nin1 mutant. FIG. 11A shows a stereo-fluorescence micrograph showing pDR5:GUS activity in hairy roots of D. glomerata under control/non-inoculated conditions. The scale bar indicates 500 μm. FIG. 11B shows a stereo-fluorescence micrograph showing pDR5:GUS activity in hairy roots of D. glomerata eight weeks after infection with clade 2 Frankia Dg1. The scale bar indicates 500 μm. FIG. 11C shows a stereo-fluorescence micrograph showing pDR5:GUS activity in hairy roots of M. truncatula under control/non-inoculated conditions. The scale bar indicates 500 μm. FIG. 11D shows a stereo-fluorescence micrograph showing pDR5:GUS activity in hairy roots of M. truncatula eight weeks after infection with S. meliloti 2011 eGFP. The scale bar indicates 500 μm. FIG. 11E shows a stereo-fluorescence micrograph showing pDR5:GUS activity in hairy roots of M. truncatula under control/non-inoculated conditions. The scale bar indicates 500 μm. FIG. 11F shows a stereo-fluorescence micrograph showing pDR5:GUS activity in hairy roots of M. truncatula eight weeks after infection with S. meliloti 2011 eGFP. The scale bar indicates 500 μm. FIG. 11G shows a stereo-fluorescence micrograph showing pDR5:GUS activity in hairy roots of M. truncatula nin1 under control/non-inoculated conditions. The scale bar indicates 500 μm. FIG. 11H shows a stereo-fluorescence micrograph showing pDR5:GUS activity in hairy roots of M. truncatula nin1 eight weeks after infection with S. meliloti 2011 eGFP. The scale bar indicates 500 μm. FIG. 11I shows a stereo-fluorescence micrograph showing pDR5:GUS activity in hairy roots of M. truncatula nin1 under control/non-inoculated conditions. The scale bar indicates 500 μm. FIG. 11J shows a stereo-fluorescence micrograph showing pDR5:GUS activity in hairy roots of M. truncatula nin1 eight weeks after infection with S. meliloti 2011 eGFP. The scale bar indicates 500p m.

FIGS. 12A-12J show that synthetic auxin responsive promoter-driven NIN affects infection in M. truncatula nin1 mutant. FIG. 12A shows a stereo-fluorescence micrograph of nin1 mutant four weeks after infection with S. meliloti 2011 pNifH-eGFP transformed with pMtNINsyn:MtNIN where infection thread progresses with the formation of a primordia. Infection is represented as a merge between bright field and eGFP (purple). FIG. 12B shows a stereo-fluorescence micrograph of nin1 mutant four weeks after infection with S. meliloti 2011 pNifH-eGFP transformed with pMtNINsyn:MtNIN where infection thread progresses with nodule formation. Infection is represented as a merge between bright field and eGFP (purple). FIG. 12C shows a stereo-fluorescence micrograph of nin1 mutant four weeks after infection with S. meliloti 2011 pNifH-eGFP transformed with pDryasNIN_5kb:DryasNIN where infection patch is visible. Infection is represented as a merge between bright field and eGFP (purple). FIG. 12D shows a stereo-fluorescence micrograph of nin1 mutant four weeks after infection with S. meliloti 2011 pNifH-eGFP transformed with pDryasNIN_5kb:DryasNIN where an extended infection thread reaching the epidermal layer is visible. Infection is represented as a merge between bright field and eGFP (purple). FIG. 12E shows a stereo-fluorescence micrograph of nin1 mutant four weeks after infection with S. meliloti 2011 pNifH-eGFP transformed with pDR5:MtNIN where infection curl is visible (arrowhead) in which bacteria failed to progress beyond the shepherd's hook. Infection is represented as a merge between bright field and eGFP (purple). FIG. 12F shows a stereo-fluorescence micrograph of nin1 mutant four weeks after infection with S. meliloti 2011 pNifH-eGFP transformed with pDR5:MtNIN where infection patch is visible. Infection is represented as a merge between bright field and eGFP (purple). FIG. 12G shows a stereo-fluorescence micrograph of nin1 mutant four weeks after infection with S. meliloti 2011 pNifH-eGFP transformed with pDR5:DryasNIN where infection curl is visible. Infection is represented as a merge between bright field and eGFP (purple, arrowhead). FIG. 12H shows a stereo-fluorescence micrograph of nin1 mutant four weeks after infection with S. meliloti 2011 pNifH-eGFP transformed with pDR5:DryasNIN where infection patch is visible. Infection is represented as a merge between bright field and eGFP (purple). FIG. 12I shows boxplots representing the number of infection threads per root for curl (left), extended (middle) and patch (right) infection thread phenotypes. The box indicates median (thick line) and second through third quartiles. Wilcoxon signed-rank test was used to determine statistical significance, shown by p-value and asterisks over brackets, where * indicates p<0.01; ** indicates p<0.002; *** indicates p<0.0003; **** indicates p<0.00006. n represents the number of transformed roots scored. The X-axis indicates the construct; the Y-axis indicates the number of infection threads of the given phenotype per root. FIG. 12J shows boxplots representing the number of nodules per root. The box indicates median (thick line) and second through third quartiles. Wilcoxon signed-rank test was used to determine statistical significance, shown by p-value and asterisks over brackets, where * indicates p<0.01; ** indicates p<0.002; *** indicates p<0.0003; **** indicates p<0.00006. n represents the number of transformed roots scored. The X-axis indicates the construct; the Y-axis indicates the number of nodules per root.

FIGS. 13A-13R show that synthetic auxin responsive promoter-driven NIN affect infection and nodulation in M. truncatula A17. FIG. 13A shows a stereo-fluorescence micrograph of an A17 plant at four weeks after infection with S. meliloti 2011 pNifH-eGFP transformed with pDR5:GUS, where infection thread formation is represented as merged bright field and eGFP (purple) (top, A), and a corresponding micrograph of eGFP+mCherry (transformation marker) (bottom, A′). FIG. 13B shows a stereo-fluorescence micrograph of an A17 plant at four weeks after infection with S. meliloti 2011 pNifH-eGFP transformed with pDR5:MtNIN, where infection thread formation is represented as merged bright field and eGFP (purple) (top, B), and a corresponding micrograph of eGFP+mCherry (transformation marker) (bottom, B′). FIG. 13C shows a stereo-fluorescence micrograph of an A17 plant at four weeks after infection with S. meliloti 2011 pNifH-eGFP transformed with pDR5:DryasNIN, where infection thread formation is represented as merged bright field and eGFP (purple) (top, C), and a corresponding micrograph of eGFP+mCherry (transformation marker) (bottom, C′). FIG. 13D shows a stereo-fluorescence micrograph of an A17 plant at four weeks after infection with S. meliloti 2011 pNifH-eGFP transformed with pDR5:DatiscaNIN, where infection thread formation is represented as merged bright field and eGFP (purple) (top, D), and a corresponding micrograph of eGFP+mCherry (transformation marker) (bottom, D′). FIG. 13E shows a stereo-fluorescence micrograph of nodules on an A17 plant at four weeks after infection with S. meliloti 2011 pNifH-eGFP transformed with pDR5:GUS, where nodules are represented as merged eGFP+mCherry. FIG. 13F shows a stereo-fluorescence micrograph of nodules on an A17 plant at four weeks after infection with S. meliloti 2011 pNifH-eGFP transformed with pDR5:MtNIN, where nodules are represented as merged eGFP+mCherry. FIG. 13G shows a stereo-fluorescence micrograph of nodules on an A17 plant at four weeks after infection with S. meliloti 2011 pNifH-eGFP transformed with pDR5:DryasNIN, where nodules are represented as merged eGFP+mCherry. FIG. 13H shows a stereo-fluorescence micrograph of nodules on an A17 plant at four weeks after infection with S. meliloti 2011 pNifH-eGFP transformed with pDR5:DatiscaNIN, where nodules are represented as merged eGFP+mCherry. FIG. 13I shows a stereo-fluorescence micrograph of A17:Enod11-GUS plants at four weeks after infection with S. meliloti 2011 pNifH-eGFP transformed with pDR5:GUS, where infection thread formation is visible at the expression site of pENOD11 representing successful primordia formation. FIG. 13J shows a stereo-fluorescence micrograph of A17:Enod11-GUS plants at four weeks after infection with S. meliloti 2011 pNifH-eGFP transformed with pDR5:MtNIN, where infection thread formation is visible at the expression site of pENOD11 representing successful primordia formation. FIG. 13K shows a stereo-fluorescence micrograph of A17:Enod11-GUS plants at four weeks after infection with S. meliloti 2011 pNifH-eGFP transformed with pDR5:DryasNIN, where infection thread formation is visible at the expression site of pENOD11 representing successful primordia formation. FIG. 13L shows a stereo-fluorescence micrograph of A17:Enod11-GUS plants at four weeks after infection with S. meliloti 2011 pNifH-eGFP transformed with pDR5:DatiscaNIN, where infection thread formation is visible at the expression site of pENOD11 representing successful primordia formation. FIG. 13M shows a stereo-fluorescence micrograph of A17:Enod11-GUS plants at four weeks after infection with S. meliloti 2011 pNifH-eGFP transformed with pDR5:GUS, where nodulation formation is visible. FIG. 13N shows a stereo-fluorescence micrograph of A17:Enod11-GUS plants at four weeks after infection with S. meliloti 2011 pNifH-eGFP transformed with pDR5:MtNIN, where nodulation formation is visible. FIG. 13O shows a stereo-fluorescence micrograph of A17:Enod11-GUS plants at four weeks after infection with S. meliloti 2011 pNifH-eGFP transformed with pDR5:DryasNIN, where nodulation formation is visible. FIG. 13P shows a stereo-fluorescence micrograph of A17:Enod11-GUS plants at four weeks after infection with S. meliloti 2011 pNifH-eGFP transformed with pDR5:DatiscaNIN, where nodulation formation is visible. FIG. 13Q shows boxplots representing number of infection threads per root for plants transformed with Empty vector (first column), pDR5:MtNIN (second column), pDR5:DryasNIN (third column), and pDR5:DatiscaNIN (fourth column). The box indicates median (thick line) and second through third quartiles, with vertical lines to minimum and maximum. The X-axis indicates the construct; the Y-axis indicates the number of infection threads per root scored. FIG. 13R shows boxplots representing number of mature nodules per root (left; blue) or number of primordia per root (right; grey) for plants transformed with Empty vector (first and fifth columns), pDR5:MtNIN (second and sixth columns), pDR5:DryasNIN (third and seventh columns), and pDR5:DatiscaNIN (fourth and eighth columns). The box indicates median (thick line) and second through third quartiles, with vertical lines to minimum and maximum. The X-axis indicates the construct; the Y-axis indicates the number of nodules per root scored. Two way ANOVA followed by Tukey-Kramer pairwise comparison was performed to calculate significant different. Significant interactions are represented as single letter codes, where different codes represent significant differences.

FIGS. 14A-14U show that auxin responsive elements (AuxRE) in the NIN promoter are essential for NIN expression. FIG. 14A shows a stereo-fluorescence micrograph of nodules of Mt nin1 mutant hairy roots transformed with pMtNINsyn-MtNIN four weeks after infection. The upper panel shows bright field and S. meliloti 2011 (pink coloration); the lower panel shows mCherry (red, transformation marker) and S. meliloti 2011 (green coloration). The scale bar=500 μm. FIG. 14B shows a stereo-fluorescence micrograph of a nodule in Mt nin1 mutant hairy roots transformed with pMtNINsyn-MtNIN four weeks after infection. The upper panel shows bright field and S. meliloti 2011 (pink coloration); the lower panel shows mCherry (red, transformation marker) and S. meliloti 2011 (green coloration). The scale bar=250 μm. FIG. 14C shows a stereo-fluorescence micrograph of primordia in Mt nin1 mutant hairy roots transformed with pMtNINsyn-MtNIN four weeks after infection. The upper panel shows bright field and S. meliloti 2011 (pink coloration); the lower panel shows mCherry (red, transformation marker) and S. meliloti 2011 (green coloration). The scale bar=250 μm. FIG. 14D shows a stereo-fluorescence micrograph of extending infection in Mt nin1 mutant hairy roots transformed with pMtNINsyn-MtNIN four weeks after infection. The upper panel shows bright field and S. meliloti 2011 (pink coloration); the lower panel shows mCherry (red, transformation marker) and S. meliloti 2011 (green coloration). The scale bar=250 μm. FIG. 14E shows a stereo-fluorescence micrograph of infection patches and curls in Mt nin1 mutant hairy roots transformed with pMtNINsyn-MtNIN four weeks after infection. The upper panel shows bright field and S. meliloti 2011 (pink coloration); the lower panel shows mCherry (red, transformation marker) and S. meliloti 2011 (green coloration). The scale bar=250 μm. FIG. 14F shows a stereo-fluorescence micrograph of Mt nin1 mutant hairy roots transformed with pMtNINsyn_AuxREmut-MtNIN four weeks after infection. The upper panel shows bright field and S. meliloti 2011 (pink coloration); the lower panel shows mCherry (red, transformation marker) and S. meliloti 2011 (green coloration). The scale bar=500 μm. FIG. 14G shows a stereo-fluorescence micrograph of a nodule in Mt nin1 mutant hairy roots transformed with pMtNINsyn_AuxREmut-MtNIN four weeks after infection. The upper panel shows bright field and S. meliloti 2011 (pink coloration); the lower panel shows mCherry (red, transformation marker) and S. meliloti 2011 (green coloration). The scale bar=250 μm. FIG. 14H shows a stereo-fluorescence micrograph of primordia in Mt nin1 mutant hairy roots transformed with pMtNINsyn_AuxREmut-MtNIN four weeks after infection. The upper panel shows bright field and S. meliloti 2011 (pink coloration); the lower panel shows mCherry (red, transformation marker) and S. meliloti 2011 (green coloration). The scale bar=250 μm. FIG. 14I shows a stereo-fluorescence micrograph of extending infection thread in Mt nin1 mutant hairy roots transformed with pMtNINsyn_AuxREmut-MtNIN four weeks after infection. The upper panel shows bright field and S. meliloti 2011 (pink coloration); the lower panel shows mCherry (red, transformation marker) and S. meliloti 2011 (green coloration). The scale bar=250 μm. FIG. 14J shows a stereo-fluorescence micrograph of infection patches and curls in Mt nin1 mutant hairy roots transformed with pMtNINsyn_AuxREmut-MtNIN four weeks after infection. The upper panel shows bright field and S. meliloti 2011 (pink coloration); the lower panel shows mCherry (red, transformation marker) and S. meliloti 2011 (green coloration). The scale bar=250 μm. FIG. 14K shows a stereomicrograph of GUS staining of lateral root primordia in an M. truncatula nin1 mutant root transformed with pDR5-GUS under control uninoculated conditions. FIG. 14L shows a stereomicrograph of GUS staining of early stages of infection of an M. truncatula nin1 root transformed with pMtNINsyn-MtNIN-pDR5-GUS four weeks after infection. FIG. 14M shows a stereomicrograph of GUS staining of mature nodule development of an M. truncatula nin1 root transformed with pMtNINsyn-MtNIN-pDR5-GUS four weeks after infection. FIG. 14N shows a stereomicrograph of GUS staining of primordia of an M. truncatula nin1 root transformed with pMtNINsyn_AuxREmut-MtNIN-pDR5-GUS four weeks after infection. FIG. 14O shows a stereomicrograph of GUS staining of more mature nodule development of an M. truncatula nin1 root transformed with pMtNINsyn_AuxREmut-MtNIN-pDR5-GUS four weeks after infection. FIG. 14P shows a stereomicrograph of GUS staining of an M. truncatula nin1::ENOD11-GUS mutant root under control uninoculated conditions. FIG. 14Q shows a stereomicrograph of GUS staining of early stages of infection of an M. truncatula nin1::ENOD11-GUS root transformed with pMtNINsyn-MtNIN four weeks after infection. FIG. 14R shows a stereomicrograph of GUS staining of mature nodule development of an M. truncatula nin1::ENOD11-GUS root transformed with pMtNINsyn-MtNIN four weeks after infection. FIG. 14S shows a stereomicrograph of GUS staining of early stages of infection of an M. truncatula nin.::ENOD11-GUS root transformed with pMtNINsyn_AuxREmut-MtNIN four weeks after infection. FIG. 14T shows a stereomicrograph of GUS staining of more mature nodule development of an M. truncatula nin1::ENOD11-GUS root transformed with pMtNINsyn_AuxREmut-MtNIN four weeks after infection. For FIGS. 14K-14T, grey shading indicates GUS staining and darker grey coloration indicates S. meliloti 2011 pNifH-GFP; the scale bar=250 μm. FIG. 14U shows bar graphs of the number of infection events per transformed root for the indicated constructs. From top to bottom, the constructs are: pMtNINsyn_AuxREmut:MtNIN-pDR5:GUS, pMtNNsyn_AuxREmut:MtNIN, pMtNINsyn:MtNIN-pDR5:GUS, and pMtNINsyn:MtNIN. From left to right, the graphs show the number of the following infection events per transformed root:top:curl, extended, patch; bottom:primordia, nodule. Significance is indicated by p-value. Statistical significance between the observations is calculated using Student's t-test followed by Bonferroni correction, where **=p<0.002 and *=p<0.02. FIG. 14V shows a ridge plot of the region between the cyclops bindings site (PACE)(grey bar at 0) and the transcriptional start site ATG, showing the position of the two putative auxin response elements (AuxRE) in the promoter of the legume NIN. The X-axis shows number of nucleotides from PACE. The TGTCTN (orange) motif is represented higher up than the TGTCTC motif (blue). Below the graph are sequences with the auxin responsive elements (AuxREs) from the wildtype promoter (pMtNINsyn, top; left: SEQ ID NO: 643, right: SEQ ID NO: 645) and a mutated form of the promoter (pMtNINsynAuxREmut, bottom; left: SEQ ID NO: 644, right: SEQ ID NO: 646).

FIGS. 15A-15E show that NIN overexpression is toxic for plants, leading to senescence. FIG. 15A shows images of a Nicotiana benthamiana leaf transiently overexpressing N-terminal tagged pLjUBI-Staygold-NIN-tNOS-p35S-mCherry-t35S, three days post infiltration. From left to right, images are brightfield, mCherry, Staygold, and UV. The scale bar=2.4 cm.

FIG. 15B shows images of a Nicotiana benthamiana leaf transiently overexpressing N-terminal tagged pMtNINsyn-GUS-tNOS-p35S-mCherry-t35S, three days post infiltration. From left to right, images are brightfield, mCherry, Staygold, and UV. The scale bar-2.4 cm. FIG. 15C shows images of a Nicotiana benthamiana leaf transiently overexpressing helper plasmid p19 three days post infiltration. From left to right, images are brightfield, mCherry, Staygold, and UV. The scale bar=2.4 cm. FIG. 15D shows a microscopy image of an N. benthamiana leaf transiently overexpressing N-terminal tagged pMtNINsyn-GUS-tNOS-p35S-mCherry-t35S (left) or N-terminal tagged pLjUBI-Staygold-NIN-tNOS-p35S-mCherry-t35S (right), three days post infiltration. The scale bar=250 μm. FIG. 15E shows box plots of the ratio of infiltrated area/senescent area for N. benthamiana leaves three days after infiltration with the indicated constructs. From top to bottom, constructs are: pLjUBI-Staygold-NIN-tNOS-p35S-mCherry-t35S, pMtNINsyn-GUS-tNOS-p35S-mCherry-t35S, and p19.

FIGS. 16A-16F show that nitrogen (N) and phosphate (P) deficiency can trigger auxin signaling in Fragaria vesca (strawberry). FIG. 16A shows a stereo-fluorescence micrograph of stable transgenic lines of F. vesca expressing pDR5:GUS under control conditions (+N+P), where GUS staining represents the readout for auxin signaling (top, A), and a magnified image of the same region (bottom, A′). The scale bar indicates 500 μm. FIG. 16B shows a stereo-fluorescence micrograph of stable transgenic lines of F. vesca expressing pDR5:GUS under conditions with exogenous auxin (+N+P+NAA), where GUS staining represents the readout for auxin signaling (top, B), and a magnified image of the same region (bottom, B′). The scale bar indicates 500 μm. FIG. 16C shows a stereo-fluorescence micrograph of stable transgenic lines of F. vesca expressing pDR5:GUS under nitrogen deficient conditions (−N+P), where GUS staining represents the readout for auxin signaling (top, C), and a magnified image of the same region (bottom, C′). The scale bar indicates 500 μm. FIG. 16D shows a stereo-fluorescence micrograph of stable transgenic lines of F. vesca expressing pDR5:GUS under nitrogen deficient conditions with exogenous auxin (−N+P+NAA), where GUS staining represents the readout for auxin signaling (top, D), and a magnified image of the same region (bottom, D′). The scale bar indicates 500 μm. FIG. 16E shows a stereo-fluorescence micrograph of stable transgenic lines of F. vesca expressing pDR5:GUS under phosphate deficient conditions (+N−P), where GUS staining represents the readout for auxin signaling (top, E), and a magnified image of the same region (bottom, E′). The scale bar indicates 500 μm. FIG. 16F shows a stereo-fluorescence micrograph of stable transgenic lines of F. vesca expressing pDR5:GUS under phosphate deficient conditions with exogenous auxin (+N−P+NAA), where GUS staining represents the readout for auxin signaling (top, F), and a magnified image of the same region (bottom, F′). The scale bar indicates 500 μm.

FIGS. 17A-17M show that DryasNIN driven by a synthetic auxin responsive promoter can trigger organogenesis of nodule-like structures (NLS) in F. vesca in the presence of auxin. FIG. 17A shows a stereo-fluorescence micrograph of stable transgenic lines of F. vesca expressing pDR5:GUS (control), treated under control conditions (+N+P) for four weeks on plates (top, A), and a corresponding micrograph of the transformation marker mCherry (bottom, A′). FIG. 17B shows a stereo-fluorescence micrograph of stable transgenic lines of F. vesca expressing pDR5:GUS (control), treated with exogenous auxin (+N+P+NAA) for four weeks on plates (top, B), and a corresponding micrograph of the transformation marker mCherry (bottom, B′). Few nodule-like structures are visible, well-spaced from one another. FIG. 17C shows a stereo-fluorescence micrograph of stable transgenic lines of F. vesca expressing pDR5:GUS (control), treated under nitrogen deficient conditions (−N+P) for four weeks on plates (top, C), and a corresponding micrograph of the transformation marker mCherry (bottom, C′). FIG. 17D shows a stereo-fluorescence micrograph of stable transgenic lines of F. vesca expressing pDR5:GUS (control), treated under nitrogen deficient conditions with exogenous auxin (−N+P+NAA) for four weeks on plates (top, D), and a corresponding micrograph of the transformation marker mCherry (bottom, D′). Few nodule-like structures are visible, well-spaced from one another. FIG. 17E shows a stereo-fluorescence micrograph of stable transgenic lines of F. vesca expressing pDR5:GUS (control), treated under phosphate deficient conditions (+N−P) for four weeks on plates (top, E), and a corresponding micrograph of the transformation marker mCherry (bottom, E′). FIG. 17F shows a stereo-fluorescence micrograph of stable transgenic lines of F. vesca expressing pDR5:GUS (control), treated under phosphate deficient conditions with exogenous auxin (+N−P+NAA) for four weeks on plates (top, F), and a corresponding micrograph of the transformation marker mCherry (bottom, F′). Few nodule-like structures are visible, well-spaced from one another. FIG. 17G shows a stereo-fluorescence micrograph of stable transgenic lines of F. vesca expressing pDR5:DryasNIN treated under control conditions (+N+P) for four weeks on plates (top, G), and a corresponding micrograph of the transformation marker mCherry (bottom, G′). FIG. 17H shows a stereo-fluorescence micrograph of stable transgenic lines of F. vesca expressing pDR5:DryasNIN, treated with exogenous auxin (+N+P+NAA) for four weeks on plates (top, H), and a corresponding micrograph of the transformation marker mCherry (bottom, H′). Numerous nodule-like structures are visible, often losing spatial distribution. FIG. 17I shows a stereo-fluorescence micrograph of stable transgenic lines of F. vesca expressing pDR5:DryasNIN treated under nitrogen deficient conditions (−N+P) for four weeks on plates (top, I), and a corresponding micrograph of the transformation marker mCherry (bottom, I′). FIG. 17J shows a stereo-fluorescence micrograph of stable transgenic lines of F. vesca expressing pDR5:DryasNIN, treated under nitrogen deficient conditions with exogenous auxin (−N+P+NAA) for four weeks on plates (top, J), and a corresponding micrograph of the transformation marker mCherry (bottom, J′). Numerous nodule-like structures are visible, often losing spatial distribution. FIG. 17K shows a stereo-fluorescence micrograph of stable transgenic lines of F. vesca expressing pDR5:DryasNIN treated under phosphate deficient conditions (+N−P) for four weeks on plates (top, K), and a corresponding micrograph of the transformation marker mCherry (bottom, K′). FIG. 17L shows a stereo-fluorescence micrograph of stable transgenic lines of F. vesca expressing pDR5:DryasNIN, treated under phosphate deficient conditions with exogenous auxin (+N−P+NAA) for four weeks on plates (top, L), and a corresponding micrograph of the transformation marker mCherry (bottom, L′). Numerous nodule-like structures are visible, often losing spatial distribution. FIG. 17M shows boxplots of primordia density (number of primordia/root length) for two independent control lines (first and second sets of six boxplots) and three independent pDR5:DryasNIN lines (third through fifth sets of six boxplots). Each set of six boxplots includes, from left to right, control conditions (+N+P), nitrogen deficient (_N+P), phosphate deficient (+N_P), exogenous auxin (+N+P+NAA), nitrogen deficient with exogenous auxin (_N+P+NAA), and phosphate deficient with exogenous auxin (+N_P+NAA). The X-axis indicates the treatment; the Y-axis indicates the primordia density (primordia/root length in cm). Two way ANOVA followed by Tukey-Kramer pairwise comparison was used to calculate significant difference. Significant interactions are represented as single letter code where different codes represent significant differences. FIG. 17N shows boxplots of lateral root number for two independent control lines (first and second boxplots of each set of five) and three independent pDR5:DryasNIN lines (third through fifth boxplots of each set of five), under the following conditions from left to right: control conditions (+N+P), nitrogen deficient (_N+P), phosphate deficient (+N_P), exogenous auxin (+N+P+NAA), nitrogen deficient with exogenous auxin (_N+P+NAA), and phosphate deficient with exogenous auxin (+N_P+NAA). The X-axis indicates the treatment; the Y-axis indicates the lateral root number. Two way ANOVA followed by Tukey-Kramer pairwise comparison was used to calculate significant difference. Significant interactions are represented as single letter code where different codes represent significant differences. FIG. 17O shows boxplots of lateral root density (number of lateral roots/root length) for two independent control lines (first and second boxplots of each set of five) and three independent pDR5:DryasNIN lines (third through fifth boxplots of each set of five), under the following conditions from left to right: control conditions (+N+P), nitrogen deficient (_N+P), phosphate deficient (+N_P), exogenous auxin (+N+P+NAA), nitrogen deficient with exogenous auxin (_N+P+NAA), and phosphate deficient with exogenous auxin (+N_P+NAA). The X-axis indicates the treatment; the Y-axis indicates the lateral root density (lateral roots/root length in cm). Two way ANOVA followed by Tukey-Kramer pairwise comparison was used to calculate significant difference. Significant interactions are represented as single letter code where different codes represent significant differences.

FIGS. 18A-18S show ultrastructure analysis of the nodule-like structures (NLS), indicating the presence of multiple vascular bundles. FIG. 18A shows a stereo-fluorescence micrograph of F. vesca roots expressing pDR5:GUS (control) treated under control conditions (+N+P) for four weeks on plates followed by clearing with 5% hypochlorite solution. The scale bar indicates 500 μm. FIG. 18B shows a stereo-fluorescence micrograph of F. vesca roots expressing pDR5:GUS (control) treated with exogenous auxin (+N+P+NAA) for four weeks on plates followed by clearing with 5% hypochlorite solution, (left, B), and the same structure under higher magnification (right, B′). The scale bar indicates 500 μm. FIG. 18C shows a section stained with Toluidine blue of F. vesca roots expressing pDR5:GUS (control) treated with exogenous auxin (+N+P+NAA) for four weeks on plates followed by clearing with 5% hypochlorite solution. The scale bar indicates 250 μm. FIG. 18D shows a stereo-fluorescence micrograph of F. vesca roots expressing pDR5:GUS (control) treated under nitrogen deficient conditions (−N+P) for four weeks on plates followed by clearing with 5% hypochlorite solution. The scale bar indicates 500 μm. FIG. 18E shows a stereo-fluorescence micrograph of F. vesca roots expressing pDR5:GUS (control) treated under nitrogen deficient conditions with exogenous auxin (−N+P+NAA) for four weeks on plates followed by clearing with 5% hypochlorite solution, (left, E), and the same structure under higher magnification (right, E′). The scale bar indicates 500 μm. FIG. 18F shows a section stained with Toluidine blue of F. vesca roots expressing pDR5:GUS (control) treated under nitrogen deficient conditions with exogenous auxin (−N+P+NAA) for four weeks on plates followed by clearing with 5% hypochlorite solution. The scale bar indicates 250 μm. FIG. 18G shows a stereo-fluorescence micrograph of F. vesca roots expressing pDR5:GUS (control) treated under phosphate deficient conditions (+N−P) for four weeks on plates followed by clearing with 5% hypochlorite solution. The scale bar indicates 500 μm. FIG. 18H shows a stereo-fluorescence micrograph of F. vesca roots expressing pDR5:GUS (control) treated under phosphate deficient conditions with exogenous auxin (+N−P+NAA) for four weeks on plates followed by clearing with 5% hypochlorite solution, (left, H), and the same structure under higher magnification (right, H′). The scale bar indicates 500 μm. FIG. 18I shows a section stained with Toluidine blue of F. vesca roots expressing pDR5:GUS (control) treated under phosphate deficient conditions with exogenous auxin (+N−P+NAA) for four weeks on plates followed by clearing with 5% hypochlorite solution. The scale bar indicates 250 μm. FIG. 15J shows a stereo-fluorescence micrograph of F. vesca roots expressing pDR5:DryasNIN treated under control conditions (+N+P) for four weeks on plates followed by clearing with 5% hypochlorite solution. The scale bar indicates 500 μm. FIG. 18K shows a stereo-fluorescence micrograph of F. vesca roots expressing pDR5:DryasNIN treated with exogenous auxin (+N+P+NAA) for four weeks on plates followed by clearing with 5% hypochlorite solution, (left, K), and the same structure under higher magnification (right, K′). The scale bar indicates 500 μm. FIG. 18L shows a section stained with Toluidine blue of F. vesca roots expressing pDR5:DryasNIN treated with exogenous auxin (+N+P+NAA) for four weeks on plates followed by clearing with 5% hypochlorite solution. Significant presence of peripheral and multiple vascular bundles is visible. The scale bar indicates 250 μm. FIG. 18M shows a stereo-fluorescence micrograph of F. vesca roots expressing pDR5:DryasNIN treated under nitrogen deficient conditions (−N+P) for four weeks on plates followed by clearing with 5% hypochlorite solution. The scale bar indicates 500 μm. FIG. 18N shows a stereo-fluorescence micrograph of F. vesca roots expressing pDR5:DryasNIN treated under nitrogen deficient conditions with exogenous auxin (−N+P+NAA) for four weeks on plates followed by clearing with 5% hypochlorite solution, (left, N), and the same structure under higher magnification (right, N′). The scale bar indicates 500 μm. FIG. 18O shows a section stained with Toluidine blue of F. vesca roots expressing pDR5:DryasNIN treated under nitrogen deficient conditions with exogenous auxin (−N+P+NAA) for four weeks on plates followed by clearing with 5% hypochlorite solution. Significant presence of peripheral and multiple vascular bundles is visible. The scale bar indicates 250 μm. FIG. 18P shows a stereo-fluorescence micrograph of F. vesca roots expressing pDR5:DryasNIN treated under phosphate deficient conditions (+N−P) for four weeks on plates followed by clearing with 5% hypochlorite solution. The scale bar indicates 500 μm. FIG. 18Q shows a stereo-fluorescence micrograph of F. vesca roots expressing pDR5:DryasNIN treated under phosphate deficient conditions with exogenous auxin (+N−P+NAA) for four weeks on plates followed by clearing with 5% hypochlorite solution, (left, Q), and the same structure under higher magnification (right, Q′). The scale bar indicates 500 μm. FIG. 18R shows a section stained with Toluidine blue of F. vesca roots expressing pDR5:DryasNIN treated under phosphate deficient conditions with exogenous auxin (+N−P+NAA) for four weeks on plates followed by clearing with 5% hypochlorite solution. Significant presence of peripheral and multiple vascular bundles is visible. The scale bar indicates 250 μm. FIG. 18S shows boxplots of primordial size for two independent control lines (first and second sets of five boxplots) and three independent pDR5:DryasNIN lines (third through fifth sets of five boxplots), under the following conditions from top to bottom: phosphate deficient with exogenous auxin (+N_P+NAA), nitrogen deficient with exogenous auxin (_N+P+NAA), exogenous auxin (+N+P+NAA), phosphate deficient (+N_P), nitrogen deficient (_N+P), and control conditions (+N+P). The X-axis indicates the treatment; the Y-axis indicates primordia area in μm. Two way ANOVA followed by Tukey-Kramer pairwise comparison was used to calculate significant difference. Significant interactions are represented as single letter code where different codes represent significant differences.

FIGS. 19A-19B show that pDR5:DryasNIN in strawberry induces nodule like structures associated with a significant increase in DryasNIN expression. FIG. 19A shows box plots of primordia area in m for control strawberry plants (‘Control’) compared to strawberry plants expressing pDR5:DryasNIN (‘pDR5:DdNIN’), grown under the following conditions from left to right: +N+P, −N+P, +N+P+NAA, and −N+P+NAA. Statistical significance between the observations is calculated using two-way ANOVA followed by Tukey-Kramer pairwise comparison test, and significant interactions are represented as single letter codes, where different codes represent significant differences. Dots of different colors represent independent lines; the data involves 3 biological replicates and error bars represent standard error of the mean. FIG. 19B shows box plots of relative expression of DryasNIN for control strawberry plants (‘Control’) compared to strawberry plants expressing pDR5:DryasNIN (‘pDR5:DdNIN’), grown under the following conditions from left to right: +N+P, −N+P, +N+P+NAA, and −N+P+NAA. Statistical significance between the observations is calculated using two-way ANOVA followed by Tukey-Kramer pairwise comparison test, and significant interactions are represented as single letter codes, where different codes represent significant differences. Dots of different shades represent independent lines; the data involves 3 biological replicates and error bars represent standard error of the mean.

FIGS. 20A-20C show RNAseq analysis of pDR5:DryasNIN strawberry lines, indicating significant number of differentially expressed genes post NAA (auxin) treatment. FIG. 20A shows volcano plots comparing RNAseq results from strawberry roots transformed with pDR5:DryasNIN and control strawberry lines grown under the indicated conditions. From top to bottom, left to right, the conditions are: +N+P, −N+P, +N+P+NAA, and −N+P+NAA. The x-axis indicates the change in expression in log₂(fold change); the y-axis indicates the level of significance as −log₁₀(adjusted p-value). Vertical dashed lines represent differentially expressed gene (DEG) thresholds (|log₂(FC)|>1.5; adj. p-value<=0.05). DEGs are indicated in darker color, with upregulated DEGs shown in red to the right of the vertical lines and downregulated genes shown in blue to the left of the downregulated lines. The number of DEGs in each direction is indicated in parentheses. FIG. 20B shows an UpSet plot of differentially expressed genes in strawberry roots transformed with pDR5:DryasNIN compared to control strawberry lines under the indicated conditions. From top to bottom, the conditions are +N+P+NAA, −N+P+NAA, −N+P, and +N+P. Bar charts represent the size of the DEG sets in each condition (lateral bar charts) and of the intersecting sets (main bar chart). Intersecting sets are shown in a matrix with the rows corresponding to the conditions and columns representing the intersection between the conditions. Intersecting conditions are connected by a solid line. FIG. 20C shows a network plot of KEGG biosynthesis of secondary metabolites indicating specific sets of pathways that are differentially affected under auxin (NAA) treatment. The colors of nodes and edges represent different functional categories. Pathways containing DEG enzymes are indicated in bold nodes and edges. The specific DEG enzyme in each pathway is indicated by a black solid line over the edges. Up-pointing triangles indicate upregulated genes, and down-pointing triangles indicate downregulated genes; red pathways and triangles indicate genes differentially expressed under −N+P+NAA conditions, and blue pathways and triangles indicate genes differentially expressed under +N+P+NAA conditions. A selection of metabolic pathways are labeled and enclosed within boxes.

FIGS. 21A-21E show RNAseq analysis of Medicago truncatula under different conditions. FIG. 21A shows a volcano plot of a mycorrhization RNAseq dataset (Mock vs Myc). The x-axis shows the expression change in log₂(fold change), and the y-axis shows the significance level as −log₁₀(adjusted P-value). Genes that are downregulated are shown to the left of the dotted vertical lines; genes that are upregulated are shown to the right of the dotted vertical lines. FIG. 21B shows a volcano plot of a lateral root development RNAseq dataset. The x-axis shows the expression change in log₂(fold change), and the y-axis shows the significance level as −log₁₀(adjusted P-value). Genes that are downregulated are shown to the left of the dotted vertical lines; genes that are upregulated are shown to the right of the dotted vertical lines. FIG. 21C shows a volcano plot of an S. meliloti nodulation RNAseq dataset. The x-axis shows the expression change in log₂(fold change), and the y-axis shows the significance level as −log₁₀(adjusted P-value). Genes that are downregulated are shown to the left of the dotted vertical lines; genes that are upregulated are shown to the right of the dotted vertical lines. FIG. 21D shows a volcano plot of a NIN overexpression RNAseq dataset. The x-axis shows the expression change in log₂(fold change), and the y-axis shows the significance level as −log₁₀(adjusted P-value). Genes that are downregulated are shown to the left of the dotted vertical lines; genes that are upregulated are shown to the right of the dotted vertical lines. FIG. 21E shows a volcano plot of a spd1 (spontaneous nodule development 1) susceptible zone RNAseq dataset. The x-axis shows the expression change in log₂(fold change), and the y-axis shows the significance level as −log₁₀(adjusted P-value). Genes that are downregulated are shown to the left of the dotted vertical lines; genes that are upregulated are shown to the right of the dotted vertical lines. For FIGS. 21A-21E vertical dashed lines represent differentially expressed gene (DEG) thresholds (|log 2(FC)|>1.5; adj. p-value<=0.05).

FIGS. 22A-22B show that gene set enrichment analysis indicates specific sets of pathways that are differentially affected in M. truncatula throughout mycorrhization and nodule development. FIG. 22A shows network plots of KEGG biosynthesis of secondary metabolites for five different RNAseq datasets—from left to right, Mock vs Myc 27 days after infection, lateral root development 72 hours post transfer, spot inoculation with S. meliloti after 24 hours, NIN overexpression, and spd1 (spontaneous nodule development) susceptible zone. Differentially expressed genes in the same pathway are joined by solid lines. The colors of nodes and edges represent different functional categories. Pathways containing DEG enzymes are indicated in bold nodes and edges. The specific DEG enzyme in each pathway is indicated by a black solid line over the edges. FIG. 22B shows a table of pathways of genes differentially expressed in M. truncatula mycorrhization and nodulation RNAseq datasets and strawberry nodule-like structure RNAseq datasets (+N+P+NAA and −N+P+NAA). Columns indicate the pathways; from left to right: AA, terpenoid backbone, monolignol biosynthesis, glavanone, gibberelin, B-carotene, castasterone, abscisic acid, sterol biosynthesis, jasmonic acid biosynthesis, riboflavin, and heme biosynthesis. Rows indicate RNAseq datasets: from top to bottom: lateral root development, AMF, spot inoculation with S. meliloti, spd1 (spontaneous nodule development 1) susceptible zone, NIN overexpression, F. vesca pDR5::DryasNIN +N+P+NAA, and F. vesca pDR5::DryasNIN −N+P+NAA.

FIGS. 23A-23F show differential expression of pDR5:GUS in barley STARTS lines post auxin (NAA) treatment. FIG. 23A shows a micrograph of barley roots of the control line before treatment. The scale bar=500 μm. FIG. 23B shows micrographs of barley roots transformed with pOsUBI-GUS (intronless) before treatment. The panel on the right (B′; scale bar=250 μm) shows the boxed region from the panel on the left (B; scale bar=500 μm) with higher magnification. FIG. 23C shows micrographs of barley roots transformed with pDR5-GUS (intronless) before treatment. The panel on the right (C′; scale bar=250 μm) shows the boxed region from the panel on the left (C; scale bar=500 μm) with higher magnification. FIG. 23D shows a micrograph of barley roots of the control line 3 weeks post treatment with 100 μM NAA. The scale bar=500 μm. FIG. 23E shows micrographs of barley roots transformed with pOsUBI-GUS (intronless) 3 weeks post treatment with 100 μM NAA. The panel on the right (E′; scale bar=250 μm) shows the boxed region from the panel on the left (E; scale bar=500 μm) with higher magnification. FIG. 23F shows micrographs of barley roots transformed with pDR5-GUS (intronless) 3 weeks post treatment with 100 μM NAA. The panel on the right (F′; scale bar=250 μm) shows the boxed region from the panel on the left (F; scale bar=500 μm) with higher magnification.

FIGS. 24A-24J show differential expression of pDR5:NLS-eGFP in barley STARTS lines post auxin (2,4D; 2,4-Dichlorophenoxyacetic acid) treatment. FIG. 24A shows CLSM (confocal laser scanning microscope) micrographs of a barley root transformed with pOsUBI::GUS under control conditions. Panel A1 shows the root on a GFP channel; Panel A2 shows the root on an mCherry channel; Panel A3 shows the root on a transmitted light channel. The scale bar=100 μm. FIG. 24B shows CLSM micrographs of a barley root transformed with pOsUBI::GUS after treatment with auxin (50 μM 2,4D). Panel B1 shows the root on a GFP channel; Panel B2 shows the root on an mCherry channel; Panel B3 shows the root on a transmitted light channel. The scale bar=100 μm. FIG. 24C shows CLSM micrographs of a barley root transformed with pOsUBI::NLS-eGFP under control conditions. Panel C1 shows the root on a GFP channel; Panel C2 shows the root on an mCherry channel; Panel C3 shows the root on a transmitted light channel. The scale bar=10 μm. FIG. 24D shows CLSM micrographs of a barley root transformed with pOsUBI::NLS-eGFP after treatment with auxin (50M 2,4D). Panel D1 shows the root on a GFP channel; Panel D2 shows the root on an mCherry channel; Panel D3 shows the root on a transmitted light channel. The scale bar=10 μm. FIG. 24E shows CLSM micrographs of a barley root transformed with pOsUBI::NLS-eGFP under control conditions. Panel E1 shows the root on a GFP channel; Panel E2 shows the root on an mCherry channel; Panel E3 shows the root on a transmitted light channel. The scale bar=10 μm. FIG. 24F shows CLSM micrographs of a barley root transformed with pOsUBI::NLS-eGFP after treatment with auxin (50 μM 2,4D). Panel F1 shows the root on a GFP channel; Panel F2 shows the root on an mCherry channel; Panel F3 shows the root on a transmitted light channel. The scale bar=10 μm. FIG. 24G shows CLSM micrographs of a barley root transformed with pDR5::NLS-eGFP under control conditions. Panel G1 shows the root on a GFP channel; Panel G2 shows the root on an mCherry channel; Panel G3 shows the root on a transmitted light channel. The scale bar=100 μm. FIG. 24H shows CLSM micrographs of a barley root transformed with pDR5::NLS-eGFP after treatment with auxin (50M 2,4D). Panel H1 shows the root on a GFP channel; Panel H2 shows the root on an mCherry channel; Panel H3 shows the root on a transmitted light channel. The scale bar=100 μm. FIG. 24I shows CLSM micrographs of a barley root transformed with pDR5::NLS-eGFP under control conditions. Panel I1 shows the root on a GFP channel; Panel 12 shows the root on an mCherry channel; Panel 13 shows the root on a transmitted light channel. The scale bar=10 μm. FIG. 24J shows CLSM micrographs of a barley root transformed with pDR5::NLS-eGFP after treatment with auxin (50 μM 2,4D). Panel J1 shows the root on a GFP channel; Panel J2 shows the root on an mCherry channel; Panel J3 shows the root on a transmitted light channel. The scale bar=10 μm.

FIGS. 25A-25O shows that pDR5:DryasNIN induces nodule-like structures in barley with auxin (NAA) treatment. FIG. 25A shows a stereo-fluorescence micrograph of wildtype barley roots under control (+N+P) conditions. The scale bar indicates 500 μm. FIG. 25B shows micrographs of wildtype barley with auxin treatment, at lower (panel B) and higher (panel B′) magnification. The scale bar indicates 500 μm. FIG. 25C shows ultrastructure analysis of nodule sections of wildtype barley stained with toluidine blue three weeks after treatment under control conditions with exogenous auxin (+N+P+NAA). The scale bar indicates 250 μm. FIG. 25D shows a micrograph of wildtype barley under nitrogen deficient conditions. The scale bar indicates 500 μm. FIG. 25E shows micrographs of wildtype barley under nitrogen deficient conditions with auxin treatment, at lower (panel E) and higher (panel E′) magnification. The scale bar indicates 500 μm. FIG. 25F shows ultrastructure analysis of nodule sections of wildtype barley stained with toluidine blue three weeks after treatment under nitrogen deficient conditions with exogenous auxin (−N+P+NAA). The scale bar indicates 250 μm. FIG. 25G shows a stereo-fluorescence micrograph of T1 barley roots transformed with pDR5-DryasNIN under control (+N+P) conditions. The scale bar indicates 500 μm. FIG. 25H shows micrographs of T1 barley roots expressing pDR5:DryasNIN under conditions with auxin treatment (+N+P+NAA), at lower (panel H) and higher (panel H′) magnification. The scale bar indicates 500 μm. FIG. 25I shows ultrastructure analysis of nodule sections of pDR5:DryasNIN barley stained with toluidine blue three weeks after treatment under control conditions with exogenous auxin (+N+P+NAA), at lower (panel I) and higher (panel I′) magnification. The scale bar indicates 250 μm. FIG. 25J shows a stereo-fluorescence micrograph of TO barley roots transformed with pDR5-DryasNIN under nitrogen deficient (−N+P) conditions. The scale bar indicates 500 μm. FIG. 25K shows micrographs of TO barley roots expressing pDR5:DryasNIN under nitrogen deficient conditions with auxin treatment (−N+P+NAA), at lower (panel K) and higher (panel K′) magnification. The scale bar indicates 500 μm. FIG. 25L shows ultrastructure analysis of nodule sections of pDR5:DryasNIN TO barley stained with toluidine blue three weeks after treatment under nitrogen deficient conditions with exogenous auxin (−N+P+NAA), at lower (panel L) and higher (panel L′) magnification. The scale bar indicates 250 μm. FIG. 25M shows a stereo-fluorescence micrograph of T1 barley roots transformed with pDR5-DryasNIN under nitrogen deficient (−N+P) conditions. The scale bar indicates 500 μm. FIG. 25N shows micrographs of T1 barley roots expressing pDR5:DryasNIN under nitrogen deficient conditions with auxin treatment (−N+P+NAA), at lower (panel N) and higher (panel N′) magnification. The scale bar indicates 500 μm. FIG. 25O shows ultrastructure analysis of nodule sections of pDR5:DryasNIN T1 barley stained with toluidine blue three weeks after treatment under nitrogen deficient conditions with exogenous auxin (−N+P+NAA), at lower (panel O) and higher (panel O′) magnification. The scale bar indicates 250 μm.

FIG. 26 shows a sequence alignment of the RWP-RK domain of members of the NIN and NLP family of proteins. The sequences from top to bottom are: M. truncatula NIN (SEQ ID NO: 554), Manihot esculenta NIN (SEQ ID NO: 571), Glycine max NIN (SEQ ID NO: 575), Datisca glomerata NINs (SEQ ID NO: 578, 579), Glycine max NIN/NLP1 orthogroup protein (SEQ ID NO: 62), Manihot esculenta NIN/NLP1 orthogroup protein (SEQ ID NO: 102), Medicago truncatula NIN/NLP1 orthogroup protein (SEQ ID NO: 109), Sorghum NIN/NLP1 orthogroup proteins (SEQ ID NOs: 183, 184), Zea mays NIN/NLP1 orthogroup proteins (SEQ ID NOs: 214, 220), Glycine max NLP2-3 orthogroup protein (SEQ ID NO: 312), Manihot esculenta NLP2-3 orthogroup protein (SEQ ID NO: 333), Medicago truncatula NLP2-3 orthogroup protein (SEQ ID NO: 336), Sorghum NLP2-3 orthogroup protein (SEQ ID NO: 360), Glycine max NLP4 orthogroup protein (SEQ ID NO: 438), Manihot esculenta NLP4 orthogroup protein (SEQ ID NO: 456), Medicago truncatula NLP4 orthogroup protein (SEQ ID NO: 460), Sorghum NLP4 orthogroup protein (SEQ ID NO: 491), and Zea mays NLP4 orthogroup protein (SEQ ID NO: 513). Positions that are 100% conserved across the alignment are indicated by asterisks (*); positions where 100% of residues are biochemically very similar are indicated by a colon (:); positions where 100% of residues are biochemically similar are indicated by a period (.).

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Genetically Modified Plants and Related Methods

In certain embodiments, the plant part may be a seed, pod, fruit, leaf, flower, stem, root, any part of the foregoing or a cell thereof, or a non-regenerable part or cell of a genetically modified plant part. As used in this context, a “non-regenerable” part or cell of a genetically modified plant or part thereof is a part or cell that itself cannot be induced to form a whole plant or cannot be induced to form a whole plant capable of sexual and/or asexual reproduction. In certain embodiments, the non-regenerable part or cell of the plant part is a part of a transgenic seed, pod, fruit, leaf, flower, stem or root or is a cell thereof.

Processed plant products that contain a detectable amount of a nucleotide segment, expressed RNA, and/or protein comprising a genetic modification disclosed herein are also provided. Such processed products include, but are not limited to, plant biomass, oil, meal, animal feed, flour, flakes, bran, lint, hulls, and processed seed. The processed product may be non-regenerable. The plant product can comprise commodity or other products of commerce derived from a transgenic plant or transgenic plant part, where the commodity or other products can be tracked through commerce by detecting a nucleotide segment, expressed RNA, and/or protein that comprises distinguishing portions of a genetic modification disclosed herein.

A control as described herein can be a control sample or a reference sample from a wild-type, an azygous, or a null-segregant plant, species, or sample or from populations thereof. A reference value can be used in place of a control or reference sample, which was previously obtained from a wild-type, azygous, or null-segregant plant, species, or sample or from populations thereof or a group of a wild-type, azygous, or null-segregant plant, species, or sample. A control sample or a reference sample can also be a sample with a known amount of a detectable composition or a spiked sample.

### Plant Breeding Methods

Plant breeding begins with the analysis of the current germplasm, the definition of problems and weaknesses of the current germplasm, the establishment of program goals, and the definition of specific breeding objectives. The next step is the selection of germplasm that possess the traits to meet the program goals. The selected germplasm is crossed in order to recombine the desired traits and through selection, varieties or parent lines are developed. The goal is to combine in a single variety or hybrid an improved combination of desirable traits from the parental germplasm. These important traits may include higher yield, field performance, improved fruit and agronomic quality, resistance to biological stresses, such as diseases and pests, and tolerance to environmental stresses, such as drought and heat.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.). Promising advanced breeding lines are thoroughly tested and compared to appropriate standards in environments representative of the commercial target area(s) for three years at least. The best lines are candidates for new commercial cultivars; those still deficient in a few traits are used as parents to produce new populations for further selection. These processes, which lead to the final step of marketing and distribution, usually take five to ten years from the time the first cross or selection is made.

The choice of breeding or selection methods depends on the mode of plant reproduction, the heritability of the trait(s) being improved, and the type of cultivar used commercially (e.g., F₁ hybrid cultivar, inbred cultivar, etc.). For highly heritable traits, a choice of superior individual plants evaluated at a single location will be effective, whereas for traits with low heritability, selection should be based on mean values obtained from replicated evaluations of families of related plants. The complexity of inheritance also influences the choice of the breeding method. Backcross breeding is used to transfer one or a few genes for a highly heritable trait into a desirable cultivar (e.g., for breeding disease-resistant cultivars), while recurrent selection techniques are used for quantitatively inherited traits controlled by numerous genes, various recurrent selection techniques are used. Commonly used selection methods include pedigree selection, modified pedigree selection, mass selection, and recurrent selection.

Pedigree selection is generally used for the improvement of self-pollinating crops or inbred lines of cross-pollinating crops. Two parents which possess favorable, complementary traits are crossed to produce an F₁. An F₂ population is produced by selfing one or several F₁s or by intercrossing two F₁s (sib mating). Selection of the best individuals is usually begun in the F₂ population; then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding (i.e., recurrent selection) may be used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs, which are also referred to as Microsatellites), Fluorescently Tagged Inter-simple Sequence Repeats (ISSRs), Single Nucleotide Polymorphisms (SNPs), Genotyping by Sequencing (GbS), and Next-generation Sequencing (NGS).

Molecular markers, or “markers”, can also be used during the breeding process for the selection of qualitative traits. For example, markers closely linked to alleles or markers containing sequences within the actual alleles of interest can be used to select plants that contain the alleles of interest. The use of markers in the selection process is often called genetic marker enhanced selection or marker-assisted selection. Methods of performing marker analysis are generally known to those of skill in the art.

Mutation breeding may also be used to introduce new traits into plant varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development: Theory and Technique, Walter Fehr (1991), Agronomy Books, 1 (https://lib.dr.iastate.edu/agron_books/1).

The production of double haploids can also be used for the development of homozygous lines in a breeding program. Double haploids are produced by the doubling of a set of chromosomes from a heterozygous plant to produce a completely homozygous individual. For example, see Wan, et al., Theor. Appl. Genet., 77:889-892, 1989.

Additional non-limiting examples of breeding methods that may be used include, without limitation, those found in Principles of Plant Breeding, John Wiley and Son, pp. 115-161 (1960); Principles of Cultivar Development: Theory and Technique, Walter Fehr (1991), Agronomy Books, 1 (https://lib[dot]dr[dot]iastate[dot]edu/agron_books/1), which are herewith incorporated by reference.

### Molecular Biological Methods to Produce Genetically Modified Plant Cells, Plant Parts, and Plants

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401-408 (1998), and Broothaerts, et al. Nature 433:629-633 (2005). The choice of method varies with the type of plant to be transformed, the particular application, and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (Kay et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466), the maize ubiquitin promoter (Christensen & Quail, Transgenic Res, 5, 213-8, 1996), the polyubiquitin promoter (Ljubql, Maekawa et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S (Franck et al., Cell (1980) 21, 285-294) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the F and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in root epidermal cells or root cortex cells. In preferred embodiments, LysM receptor promoters will be used. Non-limiting examples include NFR1 promoters, NFR5 promoters, LYK3 promoters, NFP promoters, the Lotus japonicus NFR5 promoter (SEQ ID NO: 638), the Lotus japonicus NFR1 promoter (SEQ ID NO: 642), the Medicago truncatula NFP promoter (SEQ ID NO: 639), the Lotus japonicus CERK6 promoter (SEQ ID NO: 640), and the Medicago truncatula LYK3 promoter (SEQ ID NO: 641). In additional preferred embodiments, root specific promoters will be used. Non-limiting examples include the promoter of the maize allothioneine (De Framond et al, FEBS 290, 103.-106, 1991 Application EP 452269), the chitinase promoter (Samac et al. Plant Physiol 93, 907-914, 1990), the glutamine synthetase soybean root promoter (Hirel et al. Plant Mol. Biol. 20, 207-218, 1992), the RCC3 promoter (PCT Application WO 2009/016104), the rice antiquitine promoter (PCT Application WO 2007/076115), the LRR receptor kinase promoter (PCT application WO 02/46439), the maize ZRP2 promoter (U.S. Pat. No. 5,633,363), the tomato LeExt1 promoter (Bucher et al. Plant Physiol. 128, 911-923, 2002), and the Arabidopsis pCO2 promoter (Heidstra et al, Genes Dev. 18, 1964-1969, 2004). These plant promoters can be combined with enhancer elements, they can be combined with minimal promoter elements, or can comprise repeated elements to ensure the expression profile desired.

Examples of constitutive promoters that are often used in plant cells are the cauliflower mosaic (CaMV) 35S promoter (Kay et al. Science, 236, 4805, 1987), and various derivatives of the promoter, virus promoter vein mosaic cassava (International Application WO 97/48819), the maize ubiquitin promoter (Christensen & Quail, Transgenic Res, 5, 213-8, 1996), the polyubiquitin promoter (Ljubql, Maekawa et al. Mol Plant Microbe Interact. 21, 375-82, 2008), and the Arabidopsis UBQ10 promoter (Norris et al. Plant Mol. Biol. 21, 895-906, 1993).

In some embodiments, a plant-expressible promoter can be an inducible promoter, i.e. a promoter directing a higher level of expression in response to an environmental or external cue. In some embodiments, a plant-expressible promoter can be a plant promoter that is inducible upon exposure to a plant hormone, such as auxin. Examples of inducible promoters that can be used in plant cell are the DR5 promoter (pDR5) (Ulmasov et al. Plant Cell. 9(11), 1963-71, 1997); auxin-response elements E1 promoter fragment (AuxREs) in the soybean (Glycine max L.) (Liu Plant Physiol. 115:397-407, 1997); the auxin-responsive Arabidopsis GST6 promoter (also responsive to salicylic acid and hydrogen peroxide) (Chen Plant J. 10: 955-966, 1996); and the auxin-inducible parC promoter from tobacco (Sakai 37:906-913, 1996). In some embodiments, pDR5 is used. As disclosed herein, ectopic expression of NIN downstream to the synthetic DR5 promoter resulted in spontaneous organogenesis. The potential of the pDR5:NIN was demonstrated in non-nodulating species (e.g., strawberry and barley) where stable lines expressing the construct formed nodule-like structures (NLS) in the presence of exogenous auxin. pDR5 can be combined with other promoter elements as described herein, such as minimal promoter elements, enhancer, or repeated elements to ensure the desired expression profile.

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “overexpression” refers to increased expression (e.g., of mRNA, polypeptides, etc.) relative to expression in a wild type organism (e.g., plant) as a result of genetic modification and can refer to expression of heterologous genes at a sufficient level to achieve the desired result such as increased yield. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores: 25, Max alignments: 15, and Matrix: blosum62; and for BLASTN: Gap opening penalty: 5, Gap extension penalty:2, Nucleic match: 1, Nucleic mismatch-3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions, but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

### NIN and NLP Proteins

NODULE INCEPTION (NIN) is a key regulatory transcription factor in nodulation, controlling nodule organogenesis, bacterial intracellular infection (e.g., rhizobial infection), and nodule functioning in legumes and nodulating non-legumes. NIN is part of a small gene family of NIN-LIKE PROTEINS (NLPs). Most NLP genes (including NIN orthologs in non-nodulating species) are constitutively expressed in root tissue. The encoded NLP proteins act as nitrate sensors, which upon high nitrate concentrations re-locate from the cytoplasm into the cell nucleus. In contrast, symbiotic NIN is transcriptionally induced specifically during root nodule symbiosis, and the encoded protein localizes to the cell nucleus independent of nitrate concentration. This difference underlines that the NIN protein gained essential adaptations; however, the signature of these adaptations remains unknown.

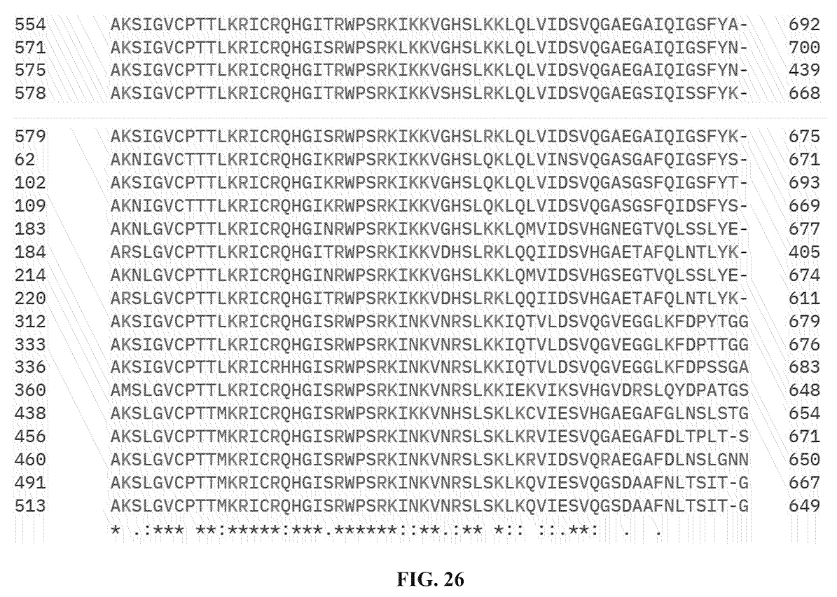

NIN and NLP proteins contain a RWP-RK domain and a Phox and Bem1 (PB1) domain (Mu and Luo (2019) Cellular and Molecular Life Sciences 76:3753-3764. Conservation of the RWP-RK domain is shown in FIG. 26. In some embodiments, the NIN protein or the NLP protein comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 649.

Exemplary NIN proteins include, but are not limited to, SEQ ID NOs: SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588. In some embodiments, a genetically modified NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588.

Exemplary NIN/NLP1 orthogroup proteins include, but are not limited to, SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236. In some embodiments, a genetically modified NIN or NLP protein is a NIN/NLP1 orthogroup protein, wherein the NIN/NLP1 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, and SEQ ID NO: 236.

Exemplary NLP2-3 orthogroup proteins include, but are not limited to, SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377. In some embodiments, a genetically modified NIN or NLP protein is a NLP2-3 orthogroup protein, wherein the NLP2-3 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377.

Exemplary NLP4 orthogroup proteins include, but are not limited to, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524. In some embodiments, a genetically modified NIN or NLP protein is a NLP4 orthogroup protein, wherein the NLP4 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524.

Exemplary basal NIN/NLP orthogroup proteins include, but are not limited to, SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550. In some embodiments, a genetically modified NIN or NLP protein is a basal NIN/NLP orthogroup protein, wherein the basal NIN/NLP orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550.

NIN or NLP can regulate, induce, promote, or increase organogenesis, nodulation, infection, neocortical cell division activity, or lateral root formation. For example, activities of NIN or NLP can include inducing nodule primordium formation. As another example, NIN or NLP can activate gibberellin biosynthesis genes because NIN activates these genes during rhizobial infection. As yet another example, NIN or NLP can induce a nodule developmental program, such as NIN activating LBD16 and YUCCA, which promotes auxin biosynthesis. This activates a nodule developmental program that overlaps with the program induced during lateral root initiation. As yet another example, NIN or NLP can have nodule initiation activity as NIN is required for nodule initiation in response to rhizobial bacteria.

Having generally described the compositions, methods, and processes of this disclosure, the same will be better understood by reference to certain specific examples, which are included herein to further illustrate the disclosure and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: Auxin Plays an Ancestral Role in Controlling the Expression of NIN During Nodule Development

The following example describes work that indicates that NIN plays an important role during intercellular actinorhizal nodulation in Datisca glomerata where DgNIN-RNAi completely abolishes nodule organogenesis. On the other hand, overexpression of D. glomerata NIN and Dryas drummondii NIN both resulted in spatial loss of lateral root and nodule distribution in D. glomerata. While promoter analysis of NIN in the actinorhizal nodulators failed to identify any cytokinin elements, auxin responsive elements were identified within −2kb of the transcription start site that were also present in all other NLPs, suggesting an ancestral switch. Ectopic expression of NIN downstream to the synthetic DR5 promoter resulted in spontaneous organogenesis in D. glomerata. Similar experiments failed to induce any organogenesis in M. truncatula but resulted in a higher number of infection events, indicating a gradual shift in the role of auxin from organogenesis in an actinorhizal context to more of an infection regulator in legumes. The potential of the DR5:NIN was proven in non-nodulating species (strawberry and barley) where stable lines expressing the construct formed nodule-like structures (NLS) in the presence of exogenous auxin. This example highlighted how phytohormonal control of NIN evolved during the stepwise evolution of nodulation and changes associated with major shifts in the bacterial symbionts.

### Introduction

Biological nitrogen fixation requires de novo organogenesis of root nodules where nitrogen fixing filamentous actinorhizal bacteria or rhizobia fix atmospheric nitrogen into ammonia. The transcription factor (TF) NIN (Nodule Inception) belonging to the NLP (NIN-Like Protein) superfamily plays the most important role in this organogenesis process. While NLPs have recently been reported for their N-sensing capability and role in lateral root organogenesis, it is apparent that the nitrogen fixing plants neo-functionalized one of the NLPs (NIN) to act as a bridge between putative NLP functions and bacterial signal perception to initiate a nodule development program.

### Materials and Methods

Growth Condition and Hairy-Root Transformation of D. glomerata

### A. Regeneration and Production of a Uniform Population of Plants

Seeds were taken out from cold storage in 1.5 ml microcentrifuge tubes and treated with 1 ml solution A (70% ethanol and 0.05% SDS) for 1 minute with gentle shaking. Following the treatment, the seeds were treated with 1 ml solution B (20% bleach and 0.1% SDS) for 5 minutes with gentle shaking. Following the treatments, the seeds were thoroughly washed with sterile deionized water for 3-4 times with gentle agitation. The seeds in the microfuge tube were resuspended in water and covered with aluminum foil and kept in the dark for 7-10 days at 4° C. After the vernalization step, the seeds were plated on ½MS (Murashige and Skoog) media with vitamins (Duchefa) pH 5.8 and 0.8% agarose (Sigma). The plates were sealed with micropore tapes and kept at 25° C. under lights with a 16 h-day and 8 h-night cycle until they germinated. Germinated seedlings were transferred to bigger plates until well established with 4-6 leaves (approx. 4-6 weeks after germination). Young fresh leaves were collected from the apical part of the plant and chopped into 4-5 mm pieces under sterile condition. The leaf pieces were transferred to Datisca Callusing Media (DCM) containing MS supplemented with 1.5 mg/L BAP (6-Benzylaminopurine), 0.1 mg/L of NAA (α-naphthaleneacetic acid), 5 g/L of Agargel™ A3301 and 30 g/L of sucrose and adjusted to pH 5.8. Leaf pieces were grown for 10-15 days before they were transferred to Datisca Regeneration Medium (DRM) petri dishes (MS-based medium supplemented with 1.5 mg/L of BAP, 0.1 mg/L of IBA (indole-3-butyric acid), 0.1 mg/L of GA3 (gibberellic acid), 5 g/L of Agargel™ A3301 and 30 g/L of sucrose and adjusted to pH 5.8. After 8-12 weeks in DRM the callus started forming shoots which were excised from the callus and moved into Datisca Rooting Medium (MS-based medium supplemented with 1 mg/L of IBA (indole-3-butyric acid), 8 g/L of Phytoagar™ and 30 g/L of sucrose and adjusted to pH 5.8. The plants started rooting within next 3-4 weeks and matured plants could be harvested for hairy-root transformation.

B. Hairy-Root Transformation of D. glomerata

For hairy root transformation, the Agrobacterium rhizogenes QUA1 harboring the plasmid of choice were grown overnight at 28° C. in LB-media with appropriate antibiotic and shaken at 220 rpm. The next day the culture was centrifuged at 4000×g and resuspended in fresh LB media before plating on LB plates containing appropriate antibiotic for selection. The plates were maintained overnight at 28° C. to get a lawn of the bacteria. Fresh D. glomerata regenerated plant shoots were harvested using a sharp sterile scalpel and inoculated with A. rhizogenes culture from the plate. The inoculated plantlets were then transferred to HMS plates supplemented with 10% sucrose (Merck), 0.5 g/L MES (Merck), and 0.8% agarose (Sigma) and adjusted to pH 5.8. The plates were sealed with micropore tapes and 90% covered with aluminum foil and kept at 22° C. for 2 days with a 16 h-day and 8 h-night cycle. After 2 days the plantlets were removed from the plates and cleaned of any excess bacterial culture and moved to new ½MS plates supplemented with 0.5 g/L MES (Merck), 0.8% agarose (Sigma) and adjusted to pH 5.8. The plants were maintained under a 16 h-day and 8 h-night cycle at 22° C. until they started forming roots which were checked for fluorescence under the stereo-fluorescence microscope DX105 F (Leica). Once the plant started forming enough fluorescent roots, the nonfluorescent roots were excised to encourage transformed root growth. After ˜4-6 weeks, the plantlets were moved into autoclaved perlite in pots and grown for another 10 days under nutrient deprived condition before they were inoculated.

C. Nodulation Assay in D. glomerata

Actively nodulating Datisca glomerata plants with Frankia clade 2 Dg1 (Persson et al., 2015) were grown in glasshouse under a 16 h-day and 8 h-night cycle at 22-24° C. The fresh nodules were harvested from the plants and were thoroughly washed with sterile water before being crushed with a mortar and pestle to form a consistent slurry. The nodule slurry was then used for inoculating the hairy-root transformed plants by dipping the roots in it. Plants were then moved onto a sterile soil mix with perlite: low nutrient peat-based soil (1:1) and maintained under a 16 h-day and 8 h-night cycle at 22-24° C. for 8-10 weeks before they were assessed for nodulation.

D. Hormone Treatment of D. glomerata

Germinated seedlings (approx. 4 weeks after germination) are moved to ½MS plates supplemented with 0.5 g/L MES (Merck), 100⁻⁶M NAA, 0.8% agarose (Sigma) and adjusted to pH 5.8. The seedlings were maintained for 10 days under a 16 h-day and 8 h-night cycle at 22-24° C. before harvesting for RNA extraction.

Growth Condition and Hairy-Root Transformation of M. truncatula

### A. Plant Material Used in the Study

M. truncatula ecotype A17 was used as a wild-type background for hairy-root transformation and for comparison to nin1 as previously described (Marsh et al., 2007). A17:Enod11-GUS lines were also used as a wild-type background to study the role of pDR5:NIN during nodulation (Marsh et al., 2007).

B. Hairy-Root Transformation of M. truncatula

Seeds were scarified with concentrated sulfuric acid in a microfuge tube for 1-2 mins until the seed coat started showing scars. Following the scarification, the sulfuric acid was thoroughly washed with sterile water for 4-5 times to remove any trace of the acid. Scarified seeds were surface sterilized using 15% (v/v) bleach solution and 1% (v/v) Tween-20 (Merck) for 15 minutes. The seeds were then thoroughly washed with sterile water and kept at 4° C. overnight in dark. The next day the seeds were plated on water droplets and allowed to germinate in darkness at 22° C. Agrobacterium rhizogenes harboring the plasmid of choice were grown overnight at 28° C. in LB-media with appropriate antibiotic and shaken at 220 rpm. The next day the culture was centrifuged at 4000×g and resuspended in fresh LB media before plating on LB plates containing appropriate antibiotic for selection. The plates were maintained overnight at 28° C. to get a lawn of the bacteria. The next day the plantlets were removed from the germination plates and hairy root transformation was done according to a previously published protocol (Boisson-Dernier et al., 2001). After the transformation, the plants were transferred to FPmod plates (Schiessl et al., 2019). The plants were maintained at 22° C. with a 16 h-day and 8 h-night cycle until they start forming transformed hairy-roots which were checked under a stereo-fluorescence microscope DX105 F (Leica).

### C. Nodulation Assay of the Hairy-Root Transformed Plants

Sinorhizobium meliloti strain 2011 (Lerouge et al., 1990) expressing pnifH:eGFP was used. The S. meliloti 2011 was grown in YEM media for 2 days at 28° C. The bacterial culture was diluted to a final concentration of 0.02 OD 600 nm using BNM (Buffered NOD) media. After maintaining the hairy-root transformed plants in nutrient deprived conditions for 3-4 days in perlite, the plants were inoculated with the S. meliloti culture (2 ml of 0.02 OD 600 nm) and maintained in perlite. Plants were grown for up to a further 4 weeks for nodule quantification and histochemical staining.

D. Hormone Treatment of M. truncatula

Germinated seedlings (approx. 4 days after germination) are moved to FPmod plates supplemented with 0.1 μM, 1 μM, or 10 μM NAA, 0.8% agarose (Sigma), and adjusted to pH 7. The seedlings were maintained for 16 hours and 2 days under 16 h-day and 8 h-night cycle at 24-22° C. before harvesting them for RNA extraction.

Raising Stable Lines of F. vesca (Strawberry)

### A. Seed Germination and Meristem Propagation

F. vesca Hawaii 4 seeds were harvested from the matured fruits and dried on filter paper at 37° C. Dried seeds were labelled and packed in envelopes and stored at 4° C. Seeds were scarified with 70% ethanol followed by 1M sulfuric acid (H₂SO₄) solution before they were thoroughly washed with water. To initiate germination, scarified seeds were plated on water agar and kept at 22° C. The germinated seedlings were propagated on nutrient rich soil in a glasshouse at 22-24° C. under long day (16 hours days-8 hours night) conditions to initiate runners. Fresh runners were harvested in water and transferred to the lab where, using a Leica stereomicroscope M165, the meristems were harvested using a scalpel. This tissue was immediately transferred to tubes containing strawberry propagation medium (SPM), taking special care to prevent desiccation. SPM is a MS-based medium (2.2 g/L) supplemented with 0.1 mg/L of BAP and 0.1 mg/L indole-3-butyric acid (IBA) and solidified with Daishin agar (Duchefa D1004, 9 g/L); the pH was adjusted to 5.8 before autoclaving. The tubes were maintained in a growth room at 20° C. under long day conditions until they regenerated into plantlets. Following shoot maturation, the plantlets were transferred to honey jars (HS French Flint Ltd, London, UK) containing SPM, where they were maintained for regular work.

### B. Plant Material and In Vitro Micropropagation

In vitro shoot cultures of F. vesca Hawaii 4 were sub-cultured at 4-6-week intervals, 5 per honey jar containing 50 ml medium. Strawberry multiplication medium (SMM) and SPM were alternated in each round of subculturing. Both basal culture media were composed of Murashige and Skoog (MS) macro and micro elements and vitamins, supplemented with sucrose (30 g/L) and 0.5 mg/L of 6-benzylaminopurine (BAP), solidified with Daishin agar (Duchefa D1004, 9 g/L) and the pH was adjusted to 5.8 before autoclaving.

### C. Transformation and Regeneration of Transgenic Plants

To prepare plant material for transformation, petioles were harvested the day before the transformation from the youngest (most apical) leaves. The plant cultures used were four to six weeks old after the last subculture.

A. tumefaciens strain EHA105 with the binary vector were grown overnight (200 rpm, 28° C.) in LB media with appropriate antibiotics. The culture was pelleted at 2,000×g for 10 minutes and re-suspended in filter-sterilized liquid MS-based medium supplemented with glucose (30 g/L) and acetosyringone (100 μM), pH 5.2, to give OD 600 nm=0.2-0.3. Petioles were cut into 4-5 mm pieces, submerged in the inoculum, and blotted on sterile filter paper to remove excess inoculum. The petiole pieces were then transferred to Strawberry Regeneration Medium (SRM) petri dishes (MS-based medium supplemented with 0.2 mg/L of α-naphthaleneacetic acid, 1 mg/L of thidiazuron (TDZ), 5 g/L of Agargel™ and 30 g/L of glucose and adjusted to pH 5.8). Petioles were co-cultivated in the dark for four days at 20° C. After the incubation, explants were washed in a solution of filter-sterilized ticarcillin disodium/clavulanate potassium (TCA, Duchefa) (400 mg/L) in water for 4 hours (60 rpm, 20° C.), then blotted and transferred to T25 Cell Culture Flasks (Nunc) containing 15 ml of liquid SRM with antibiotic selection. Flasks were placed in a shaker at 60 rpm, 20° C., under low light intensity for 4 weeks, and then blotted and transferred to SRM selection petri dishes. Petioles were sub-cultured every 4 weeks until regeneration. Control (WT) shoots were regenerated using the same method, except that the explants were not co-cultivated with A. tumefaciens and selection antibiotics were omitted from the culture media. The transformed shoots were transferred to 30 ml universal tubes (Fisher Scientific) containing 15 ml of rooting medium (Frag R) with selection. Frag R is MS-based medium (2.2 g/L) supplemented with 0.1 mg/L of BAP and 0.1 mg/L IBA and 20 g/L of glucose, solidified with 9 g/L of Daishin agar (Duchefa) and adjusted to pH 5.8. After 4 weeks, shoots were moved to tubes containing SMT medium (MS-based medium supplemented with 0.225 mg/L of BAP, 0.2 mg/L IBA, 0.1 mg/L gibberellic acid (GA3) and 30 g/L of glucose, solidified with 7.5 g/L of Sigma-Aldrich agar and adjusted to pH 5.6 before autoclaving). In the next subculturing step, plants were changed to SMM tubes.

After 4 weeks in SMM tubes, plants were mature enough to be moved to honey jars (5 plants per jar). Honey jars with SMM medium or Strawberry Medium for Rooting (SMR) were alternated at 4-6-week intervals. SMR medium is a MS-based medium supplemented with 0.4 mg/L of IBA, 0.1 mg/L gibberellic acid (GA3) and 30 g/L of glucose, solidified with 7.5 g/L of Sigma-Aldrich agar A1296 and adjusted to pH 5.6 before autoclaving.

### D. Genotyping of Transgenic Lines

DNA were extracted from 50-100 mg of leaf tissue using the following protocol. The frozen leaf tissue was ground with metal ball-bearing balls (IG100_5/32_PK1000; Simply Bearings Ltd., Leigh, UK) using a mechanical pulverize (MiniG® from Spex) at 1200 rpm for 30 seconds. 500 μl of the extraction buffer (1.25% sodium dodecyl sulfate; 100 mM Tris HCl pH 8.0; 50 mM EDTA pH 8.0 and 25 mg PVP) were added to the disrupted tissue. The samples were mixed and incubated at 65° C. for 30 minutes, inverting the tubes every 5 minutes. Samples were cooled by placing them in ice for around 5 minutes and then 250 μl of chilled 5M NaCl, mix and incubate in ice for 15 more min. The samples were centrifuged for 10 minutes at 20,000×g. Supernatant was transferred into a new tube containing 360 μl of isopropanol. Samples were vortexed and incubated for 30 minutes or overnight at −20° C. to allow DNA to precipitate. The samples were centrifuged for 20 minutes at 15,700×g. Supernatant was discarded and pellet was washed in 500 μl of 70% ethanol. The samples were centrifuged for 20 minutes at 15,700×g and supernatant was discarded. Washing step was repeated once more and supernatant was discarded. Each pellet was resuspended in 50 μl TE buffer (10 mM Tris HCl pH 8.0; 1 mM EDTA pH 8.0). PCR amplification was performed using gene specific primers and PCRBIO Taq Mix Red (PCR Biosystems) following the manufacturer's guidelines.

E. Hormone Treatment of F. vesca

Tissue culture plants growing in SMR (approximately 4 weeks after transfer) were moved to ½ Hoagland's media plates supplemented with 100⁻⁶M NAA, 0.8% agarose (Sigma). The plants were maintained for 4 weeks under a 16 h-day and 8 h-night cycle at 22-24° C. before harvesting for phenotyping and RNA extraction.

### Construct Production

The Golden Gate modular cloning system was used to prepare the plasmids for the transformation experiments (Weber et al., 2011). All the Level 0, Level 1, and Level 2 constructs are listed in Tables 1-3, respectively. Sequences were domesticated, synthesized, and cloned into pMS (GeneArt, Thermo Fisher Scientific, Waltham, USA). Sequence information was obtained for MtNIN (M. truncatula Mt4.0v1 genome via Phytozome (phytozome[dot]jgi[dot]doe[dot]gov)) (Goodstein et al., 2012) and DgNIN (D. glomerata (ASM325502v1)) and DryasNIN (D. drummondii (ASM325486v1)) from NCBI (Griesmann et al., 2018). For the DgNIN-RNAi construct the fragment was amplified using forward primers 5′-ccgggattccgctatggtcagcaggttgatg-3′ (SEQ ID NO: 647) with BamH1 overhang and reverse primer 5′-cgcctcgagcagaatgcttgtaacacccag-3′ (SEQ ID NO: 648) with Xho1 overhang and cloned into entry vector pENTR3c dual (Invitrogen) and sequenced for confirmation. The entry clone was recombined into the destination vector pK7GWIWG2-7F2.1 (VIB, Ghent) using the LR clonase Gateway cloning kit (Invitrogen) (see Table 2 and Table 3).

TABLE 1
Level 0 construct assembly sheet.
PU = promoter; SC = coding region; T = terminator.

| EC | |
|---|---|
| number | Construct details |
| EC37362 | pL0M-PU-pDatiscaNIN_F1-5-37362 |
| EC54188 | pL0B-PU-pDatiscaNIN2_F1-5-54188 |
| EC37363 | pL0-PU-pDryasNIN_F1-5-37363 |
| EC55328 | pL0M-PU-pMtNIN(3C) + 3XMtCYC + 35S-55328 |
| EC25116 | pL0M-PU-pDR5 |
| EC15057 | pL0M-PU-pNOS-15057 |
| EC51266 | pL0M-PU-35S-TMV-1-51266 |
| EC15251 | pL0M-PU-LjUBI-15251 |
| EC54611 | pL0B-PU-pMtNINsyn-54611 |
| EC54614 | pL0B-PU-pMtNINsynAuxREmut-54614 |
| EC54535 | pL0M-S-n2-Staygold-CO-54535 |
| EC75111 | pL0M-SC-GUS-intron-75111 |
| EC15068 | pL0M-SC-Kan-15068 |
| EC15071 | pL0M-SC-mCherry-15071 |
| EC61119 | pL0M-SC-MtNIN(CDS)-61119 |
| EC54016 | pL0B-SC-DgNIN_cDNA-F1-3(CDS)-54016 |
| EC54189 | pL0B-SC-DatiscaNIN2-cDNA_F1-2-54189 |
| EC54041 | pL0B-SC-Dryas_drumNIN_cDNA_F1-3(CDS)-54041 |
| EC61120 | pL0M-C-MtNIN(CDS)-61120 |
| EC15318 | pL0M-T-Rbcs-15318 |
| EC41414 | pL0M-T-35S-1-41414 |
| EC41421 | pL0M-T-Nos-41421 |
| EC41432 | pL0M-T-ocs-1-41432 |

TABLE 2
Level 1 construct assembly sheet.

| EC | |
|---|---|
| number | Construct details |
| EC15029 | pL1M-R1-pNOS-Kan-tNOS-15029 |
| EC54091 | pL1B-R3-p35S-mcherry-t35S-54091 |
| EC54007 | pL1B-R2-pDatiscaNIN-GUS-tocs-54007 |
| EC54115 | pL1B-R2-pDryasNIN-GUS-tNOS-54115 |
| EC54019 | pL1B-R2-pDR5-GUS-tNOS-54019 |
| EC54021 | pL1B-R2-pDR5-DgNIN-tNOS-54021 |
| EC54197 | pL1B-R2-pDR5-DdNIN_cDNA-tNOS-54197 |
| EC54118 | pL1B-R3-pLjUBI-3xHA-DdNIN-trbcs-54118 |
| EC54562 | pL1B-R3-pLjUBI-DgNIN-FLAG-tNOS-54562 |
| EC54541 | pL1B-R2-pMtNIN(3C) + 3XMtCYC + 35S-GUS-tNOS- |
| | 54541 |
| EC54542 | pL1B-R2-pDR5-MtNIN_CDS-tNOS-54542 |
| EC54543 | pL1B-R2-pMtNIN(3C) + 3XMtCYC + 35S-MtNIN_CDS- |
| | tNOS-54543 |
| EC54544 | pL1B-R2-pMtNIN(3C) + 3XMtCYC + 35S-DgNIN_CDS- |
| | tNOS-54544 |
| EC54545 | pL1B-R2-pMtNIN(3C) + 3XMtCYC + 35S-DdNIN_CDS- |
| | tNOS-54545 |
| EC54327 | pL1B-R2-pDdNIN-DdNIN-tNOS-54327 |
| EC54264 | pDgNIN-RNAi pENTR3c-54264 |
| EC54621 | pL1B-R2-pMtNINsyn-MtNIN-tNOS-54621 |
| EC54622 | pL1B-R2-pMtNINsynAuxREmut-MtNIN-tNOS-54622 |
| EC54574 | pL1B-R2-pLjUBI-Staygold-MtNIN-tNOS-54574 |
| EC54198 | EC54198 pL1B-R2-pDgNIN2-GUS-tOcs-54198 |
| EC54564 | EC54564 pL1B-R3-pLjUBI-DgNIN2-FLAG-tNOS-54564 |

TABLE 3
Level 2 construct assembly sheet.

| | | Transformed plants | | | |
|---|---|---|---|---|---|
| EC | | D | M. | F. | H. |
| number | Construct details | glomerata | truncatula | vesca | vulgare |
| EC54010 | EC54010 pL2B-Kan-pDgNIN-GUS- | X | X | | |
| | tNOS-p35S-mCherry-t35S-54010 | | | | |
| EC54119 | EC54119 pL2B-Kan-pDdNIN-GUS- | X | X | | |
| | tNOS-p35S-mCherry-t35S-54119 | | | | |
| EC54096 | EC54096 pL2B-Kan-pDR5-GUS-tNOS- | X | X | X | |
| | p35S-mCherry-t35S-54096 | | | | |
| EC54097 | EC54097 pL2B-Kan-pDR5- | X | | | |
| | DgNIN_cDNA-tNOS-p35S-mCherry- | | | | |
| | t35S-54097 | | | | |
| EC54208 | EC54208 pL2B-Kan-pDR5- | X | X | X | |
| | DdNIN_cDNA-tNOS-p35S-mCherry- | | | | |
| | t35S-54208 | | | | |
| EC54123 | EC54123 pL2B-Kan-pLjUBI-DdNIN- | X | | | |
| | 3xMyc-trbcs-p35S-mCherry-t35S- | | | | |
| | 54123 | | | | |
| EC54570 | EC54570 pL2B-Kan-p35S-mCherry- | X | | | |
| | t35S-pLjUBI-DgNIN-FLAG-tNOS- | | | | |
| | 54570 | | | | |
| EC54548 | EC54548 pL2B-Kan- | | X | | |
| | pMtNIN(3C) + 3XMtCYC + 35S-GUS- | | | | |
| | tNOS-p35S-mCherry-t35S-54548 | | | | |
| EC54549 | EC54549 pL2B-Kan-pDR5- | | X | | |
| | MtNIN_CDS-tNOS-p35S-mCherry- | | | | |
| | t35S-54549 | | | | |
| EC54550 | EC54550 pL2B-Kan- | | X | | |
| | pMtNIN(3C) + 3XMtCYC + 35S- | | | | |
| | MtNIN_CDS-tNOS-p35S-mCherry- | | | | |
| | t35S-54550 | | | | |
| EC54551 | EC54551 pL2B-Kan- | | X | | |
| | pMtNIN(3C) + 3XMtCYC + 35S- | | | | |
| | DgNIN_CDS-tNOS-p35S-mCherry- | | | | |
| | t35S-54551 | | | | |
| EC54552 | EC54552 pL2B-Kan- | | X | | |
| | pMtNIN(3C) + 3XMtCYC + 35S- | | | | |
| | DdNIN_CDS-tNOS-p35S-mCherry- | | | | |
| | t35S-54552 | | | | |
| EC54325 | EC54325 pL2B-Kan-pDdNIN-DdNIN- | | X | | |
| | tNOS-p35S-mCherry-t35S-54325 | | | | |
| EC54272 | EC54272 pK7GWIWG2-7F2.1-Kan- | X | | | |
| | p35S-DgNIN-RNAi-t35S-p35S-eGFP- | | | | |
| | t35S-54272 | | | | |
| EC54623 | pL2B-Kan-pMtNINsyn-MtNIN-tNOS- | | X | | |
| | p35S-mCherry-t35S-54623 | | | | |
| EC54624 | pL2B-Kan-pMtNINsynAuxREmut- | | X | | |
| | MtNIN-tNOS-p35S-mCherry-t35S- | | | | |
| | 54624 | | | | |
| EC54634 | pL2B-p35S-mCherry-t35S-pMtNINsyn- | | X | | |
| | MtNIN-tNOS-pDR5-GUS-trbcs-54634 | | | | |
| EC54635 | pL2B-p35S-mCherry-t35S- | | X | | |
| | pMtNINsynAuxREmut-MtNIN-tNOS- | | | | |
| | pDR5-GUS-trbcs-54635 | | | | |
| EC54338 | pL2B-Hyg-pDR5-DgNIN_cDNA- | | | | X |
| | tNOS-p35S-mCherry-t35S-54338 | | | | |
| EC70781 | pL2B-nptII-pZmUBI-mCherry- | | | | X |
| | pDR5::GUS-70781 | | | | |
| EC70785 | pL2B-nptII-pZmUBI-mCherry- | | | | X |
| | pDR5::NLS_eGFP-70785 | | | | |
| EC54203 | pL2B-Kan-pDgNIN2-GUS-tOcs-p35S- | X | | | |
| | mCherry-t35S-54203 | | | | |
| EC54572 | pL2B-Kan-p35S-mCherry-t35S- | X | | | |
| | pLjUBI-DgNIN2-FLAG-tNOS-54572 | | | | |

TABLE 4
Primers used.

| | | SEQ ID |
|---|---|---|
| Primer name | Sequence | NO |
| DgActin qRT-for | ggaatggaagc | 632 |
| | tgctggaatc | |
| DgActin qRT-rev | ggtctgcaata | 633 |
| | cctgggaac | |
| DgNIN qRT-for | ggttagaggta | 634 |
| | acagactcatc | |
| | cattc | |
| DgNIN qRT-rev | caggatcttca | 635 |
| | tgatcttggta | |
| | gattg | |
| DgNIN2 qRT-for | caaggacgttg | 636 |
| | gatggattcct | |
| | caaatgg | |
| DgNIN2 qRT-rev | cgtgattggcc | 637 |
| | tgatcttcttc | |
| | gtag | |

### Gene Expression Analysis

Each plant root system consisting of transformed root or nodules was harvested and each individual root system formed a biological replicate; 3 or more technical replicates were prepared from each root system having a 100-150 mg sample. RNA was extracted using a modified extraction buffer containing 2% SDS (v/v), 100 mM Tris-HCl pH 8.0, 1.4M NaCl, and 20 mM EDTA. The samples were crushed with tungsten-carbide beads (Qiagen) in a mechanical pulverize (Spex Geno/Grinder®) in the presence of 12 μl/ml β-mercaptoethanol (Sigma) and 50 mg PVPP (Sigma). The powder was resuspended with the extraction buffer followed by treatment with chloroform:iso-amyl alcohol (24:1)(Melford) and chloroform (Sigma). The RNA was precipitated using 7.5M Lithium Chloride solution. The precipitated RNA was washed with 75% ethanol followed by air drying and re-suspension in deionized water. The extracted RNA was quantified using a NanoDrop® (Thermo) and then treated for DNase digestion using TURBO DNA-free™ kit (Thermo) using the manufacturer protocol. 500 ng of the treated RNA was then reverse transcribed to make cDNA using Superscript™ III (Invitrogen) using manufacturer protocol. Quantitative real-time polymerase chain reaction (qRT-PCR) was performed in technical replicates using SyGreen mix Lo-ROX (PCR Biosystems) using QuantStudio™ 7 Flex system (ABI) in a total volume of 10 μl. The primer pairs for gene expression analysis are listed in Table 4.

### Histochemical Assays and Staining

For GUS staining the harvested roots were thoroughly cleaned in sterile water and scored under the stereo-fluorescent microscope so that only the transformed roots were harvested for the staining purpose. The roots were fixed in 90% acetone on ice for 1 hour. Subsequently, the acetone was replaced with 50 mM phosphate buffer pH7.2 (Sigma) and the roots were washed twice to remove any acetone. The roots were then moved to the staining solution containing 0.1M Phosphate buffer, 0.25 mM K₃[Fe(CN)₆](potassium ferricyanide), 0.25 mM K₄[Fe(CN)₆](potassium ferrocyanide), 0.25% (v/v) Triton X-100 and 1 mM 5-bromo-4-chloro-3-indolyl beta-D-glucuronide (X-Gluc, Thermo). The samples were vacuum infiltrated for 30 minutes and incubated at 37° C. overnight. After staining the tissues were cleared of all the staining solution and stored in 50% ethanol for imaging under the stereomicroscope DX105 F (Leica). For ruthenium red staining the staining solution was prepared (0.1% ruthenium red in sterile water) and a few microliters are added over each section and subsequently washed using sterile deionized water. For toluidine blue staining, 0.1% toluidine blue staining solution was prepared in deionized water and a few microliters are added over each section and subsequently washed using sterile deionized water.

### Tissue Sections

Roots and nodules are fixed with 2.5% glutaraldehyde (Sigma) in 1×PBS overnight. The fixed material was dehydrated in an ethanol series and subsequently embedded in Technovit® 7100 (Kulzer Technik, Wehrheim, Germany) according to the manufacturer protocol. Embedded tissues were sectioned (10-15 μm) using a Leica microtome (HistoCore AUTOCUT®) and mounted on glass slides followed by staining with ruthenium red solution or toluidine blue staining solution. Tissue sections were imaged using a stereomicroscope DX105 F (Leica) using LAS 3.4 software.

### Phenotypic Analysis

A. M. truncatula Hairy-Root Experiments

To quantify the total number of curled root hair, extended root hair, and infection patch, each transgenic root was considered as an independent system and all the structures were scored in random sections of ˜1 cm according to Liu et. al., 2019. The nodules were counted in all the transformed roots using a stereo microscope DX105 F (Leica) using LAS 3.4 software.

B. D. glomerata Hairy-Root Experiments

Whole plants were imaged using ImageQuant™ 800 platform (Cytiva) with proprietary software and final images are processed using Photoshop 2024. The higher magnification images were captured using a stereo microscope DX105 F (Leica) using LAS 3.4 software.

C. F. vesca Whole Transgenics

Whole plants were imaged using a stereo microscope DX105 F (Leica) using LAS 3.4 software.

### Phylogenetic Analysis and Prediction of Auxin Binding Sites

NIN homologues were searched using the reciprocal BLAST function in Geneious v2021.1.1 against the publicly available genomes of Medicago truncatula (Tang et al., 2014), Lotus japonicus (Sato et al., 2008), Vigna unguiculata (LIS), Arachis (Bertioli et al., 2016), Cicer (Varshney et al., 2013), Cajanus (Varshney et al., 2012), Glycine (Schmutz et al., 2010), Parasponia (van Velzen et al., 2018); Alnus, Discaria (Ocheotphila), Dryas, Datisca, Casuarina (Griesmann et al., 2018); Cercocarpus (this study), Ceanothus (this study) and Lupin (Hane et al., 2017). The NIN proteins were aligned in MEGA10 using clustalW, and the alignment was subsequently used to prepare the ML-tree. Based on the transcriptional start site, the cyclops binding site was identified in the upstream promoter region. In the extracted genomic sequences the putative auxin responsive elements (AuxREs) were identified and the positions were plotted in R using the ggridges 0.5.4 package (Wilke, 2022).

### RNA Extractions

RNA was extracted from 250-500 mg of root tissue of D. glomerata, M. truncatula, and F. vesca.

### RNAseq Analysis

RNA integrity was assessed using the Agilent TapeStation system using RNA screen tape. Library preparation and paired-end RNA sequencing was performed on an Illumina NovaSeq™ 6000 platform.

Raw reads were quality controlled using FastQC v0.11.9 (S. Andrews, 2010), and adapters and low-quality regions were trimmed using Kallisto using a sliding window of 4 and minimum PHRED score of 20 (Bray et al., 2016). The first 10 nucleotides were trimmed and reads less than 100 nucleotides and unpaired reads were discarded. Assemblies were indexed and reads were aligned using Kallisto using the default settings for paired end reads (Bray et al., 2016). Quantification was performed using featureCounts v2.0.1 and differential expression analysis was performed with the R package DESeq2 v3.17 (Liao et al., 2014; Love et al., 2014).

### Realtime PCR

Total RNA was reverse-transcribed using SuperScript™ III Reverse Transcriptase (Life Technologies) and anchored dT17 primers as described in the manufacturer's protocol. About 1 μl of cDNA was used to set up real-time RT-qPCR in a 20-1 reaction system with 2×SYBR™ Green PCR master mixes (PCR biosystems) using an ABI Proflex7 Real-Time PCR system. PCR program: 1 cycle at 50° C. for 2 min, 1 cycle at 95° C. for 5 min, and 40 cycles at 95° C. for 30 s and 60° C. for 30s. Expression data were obtained from 3 independent biological repetitions. Calculations were done using the cycle threshold method using MtEF1a, DgActin and FvHistoneH4 as the endogenous control.

### Results and Discussion

### Phylogenetically Actinorhizal NIN is Closer to the NLPs in Retaining the Nitrate Sensing Domain.

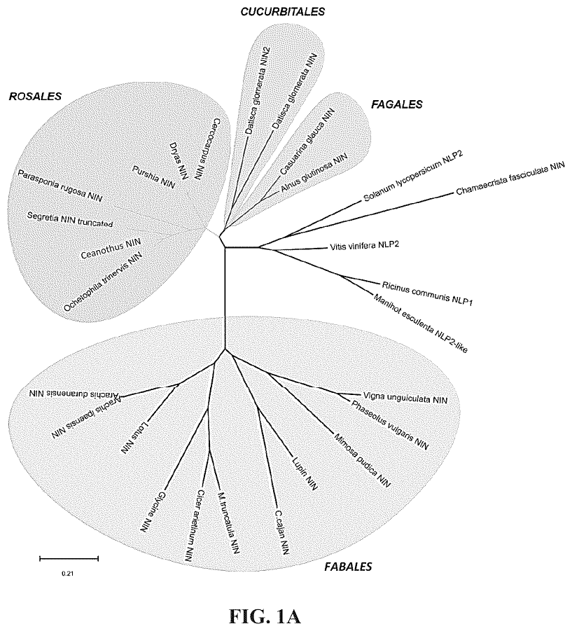

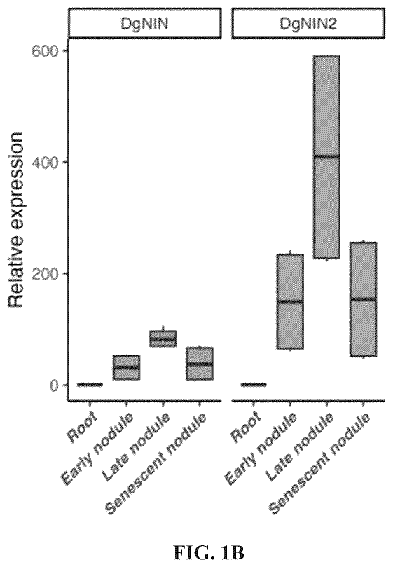

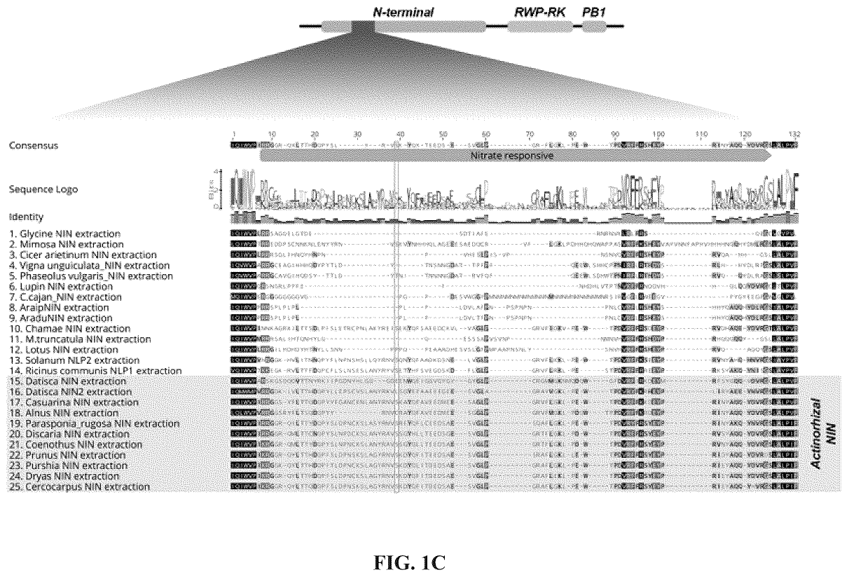

NIN belongs to a bigger family of transcription factors known as NIN like-proteins (NLPs) which is ubiquitously present in land plants starting from green algae (Chlamydomonas reinhardtii), red algae (Porphyra umbilicalis), and liverworts (Marchantia polymorpha) to eudicots and monocots (Mu & Luo, 2019). The number of NLPs varies considerably depending upon whether the plant lineage experienced any duplication or triplication event during evolution, as seen in poplar (Tuskan et al., 2006). NIN was first reported from Lotus japonicus where it was found to play an important role in nodule development (Schauser et al., 1999). Comparative genomics study of plants within the nitrogen fixation clade (NFN) indicated that NIN was only retained in the functional nodulators and any loss or pseudogenisation resulted in complete loss of symbiotic nitrogen fixation capability (Griesmann et al., 2018; van Velzen et al., 2018). Whole genome sequencing of the actinorhizal plants like Cercocarpus, Ceanothus and Purshia indicated the presence of a single copy of full-length NIN (FIG. 1A, Rosales clade). At the same time, the presence of two copies of NIN were also identified in Datisca glomerata (DgNIN1 (also referred to as DgNIN) and DgNIN2) (FIG. 1A, Cucurbitales clade). The presence of 2 copies of NIN is not unique to D. glomerata as similar NIN duplication was also reported for the Dalbergoid legume Arachis hypogaea (Bhattacharjee et al., 2022). Expression analysis of both the NIN copies indicates that they are highly expressed in the symbiotic nodules indicating their potential role in intercellular actinorhizal nodule development in D. glomerata (FIG. 1B).

NIN codes for a transcription factor with a N-terminal region whose role during nodule development has yet to be determined, followed by the DNA binding RWP-RK motif and the protein-protein interacting PB1 domain (FIG. 1C, top) (Schauser et al., 1999). It has recently been found that the N-terminal region of the NLP has a nitrate sensing role and subsequently was termed the nitrate responsive domain (NRD) (Konishi & Yanagisawa, 2013). The actinorhizal NIN proteins, including DgNIN1 and DgNIN2, show significant differences in the N-terminal region as compared to the legume NINs (FIG. 1C, bottom). One major difference is the retention of the conserved serine residue in the NRD for the actinorhizal NINs which is lost in the legume NINs except for the Chamaecrista (FIG. 1C). The conserved 5205 in Arabidopsis NLP7 is essential for phosphorylation by CPK and its retention in the nucleus (K. H. Liu et al., 2017). The loss of the serine in legume is often regarded as an evolutionary shift for the NIN to take a role in nitrogen fixation from being a nitrate sensor in the distant ancestor (Suzuki et al., 2013). Thus, the homology of the actinorhizal NINs to the NLPs indicated their ancestral adaptation as a protein with a possible dual role in nitrogen fixation as well as being a nitrate sensor. A similar observation was made in poplar, where 4 out of the 5 full length NINs retained the serine residue (Irving et al., 2022).

### Spatiotemporal Expression Pattern of NIN During Intercellular Actinorhizal Nodule Development.

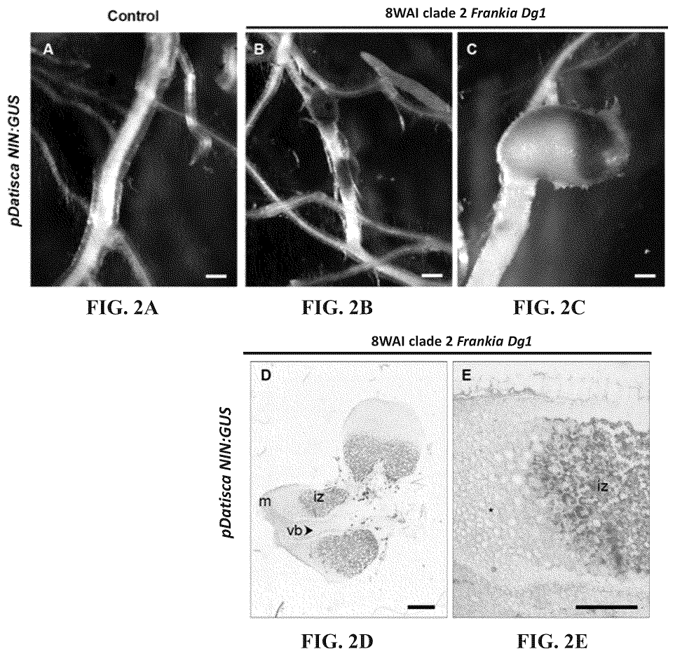

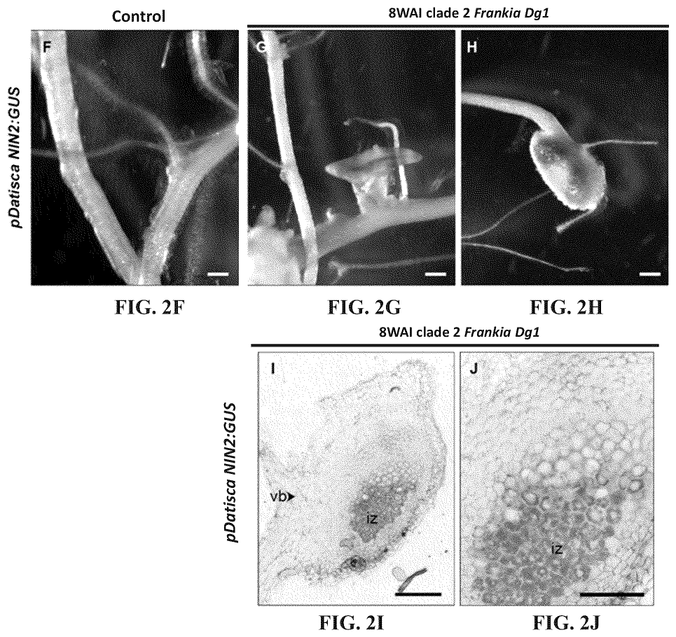

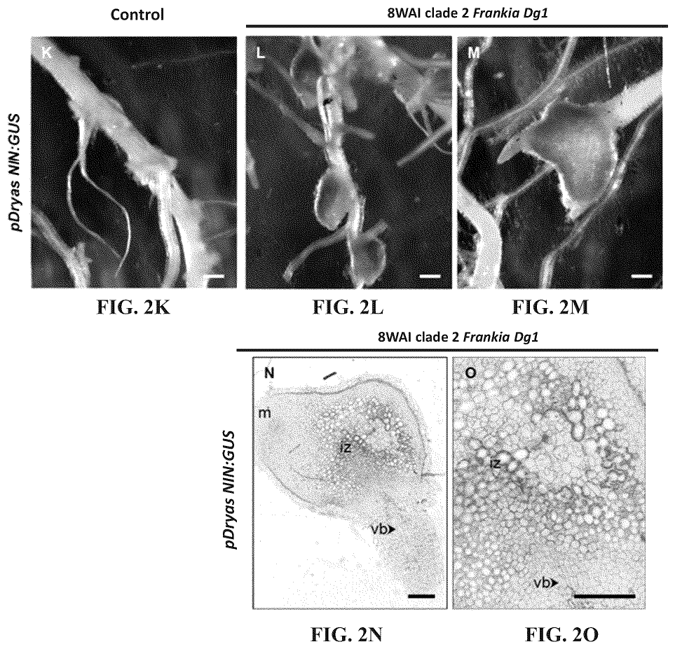

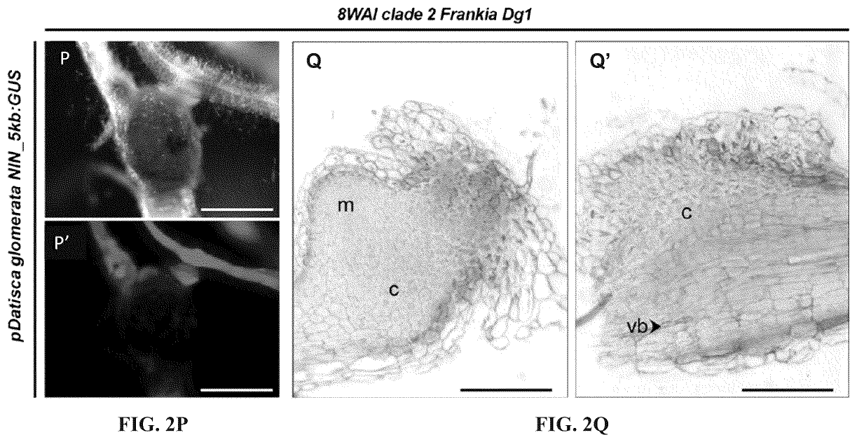

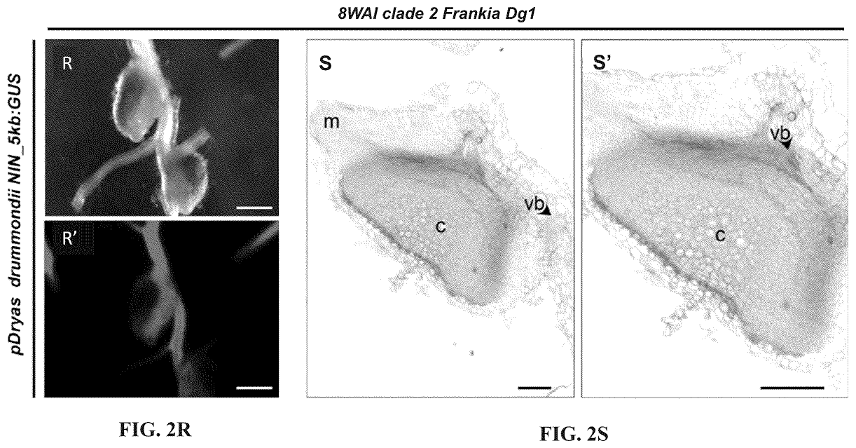

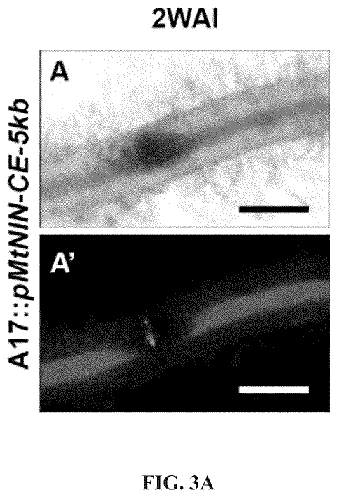

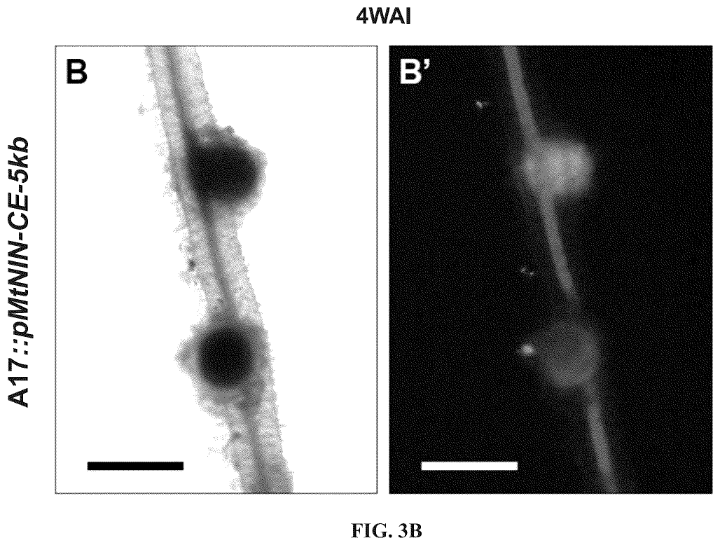

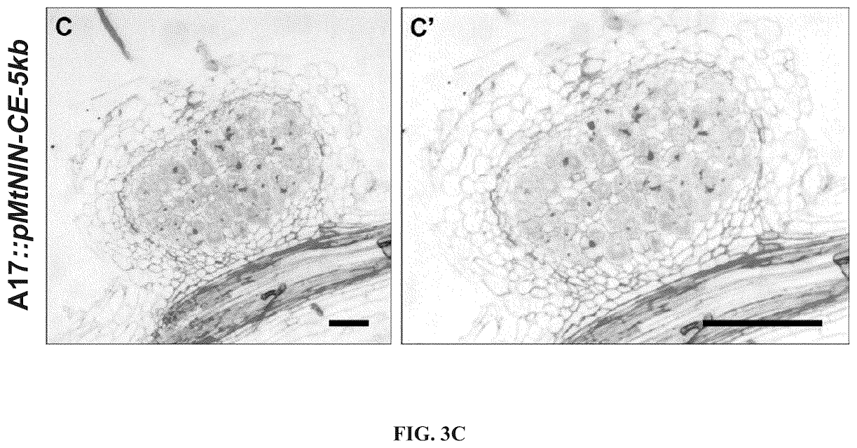

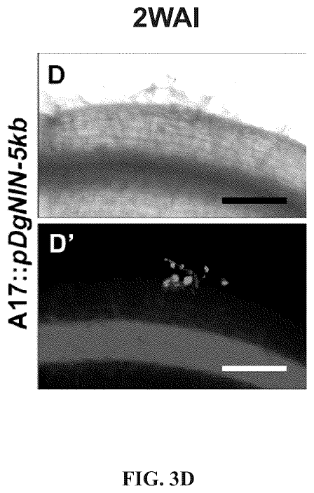

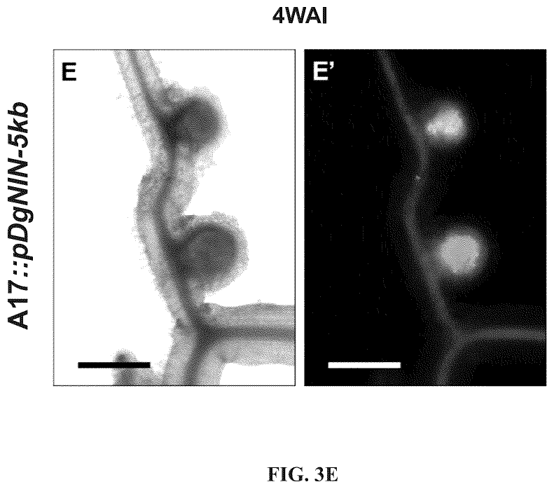

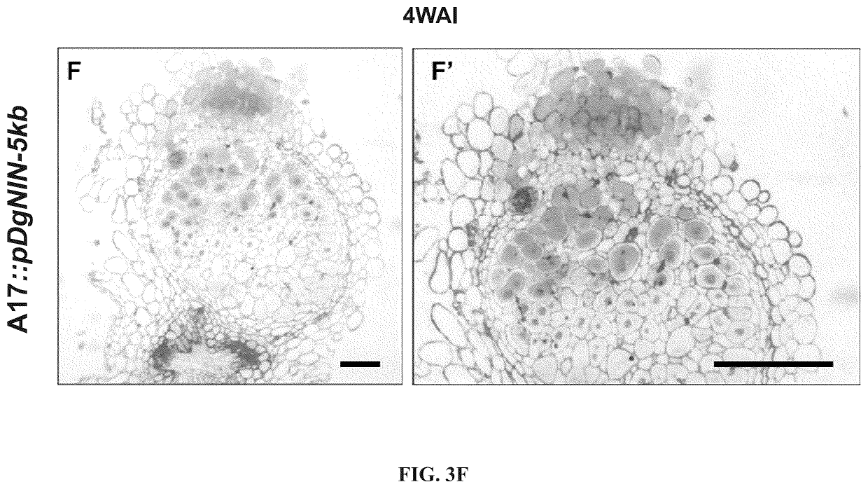

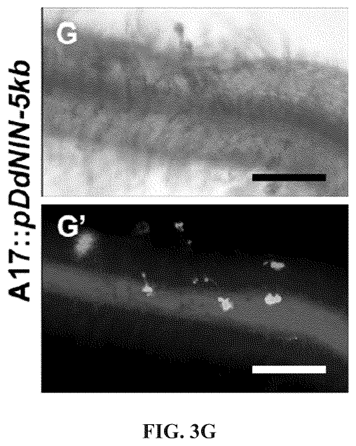

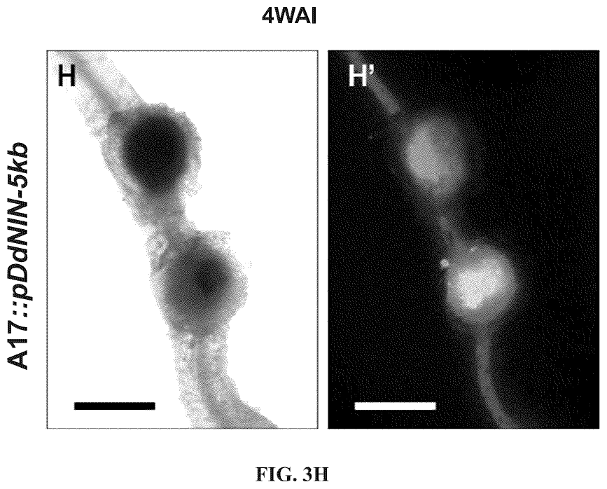

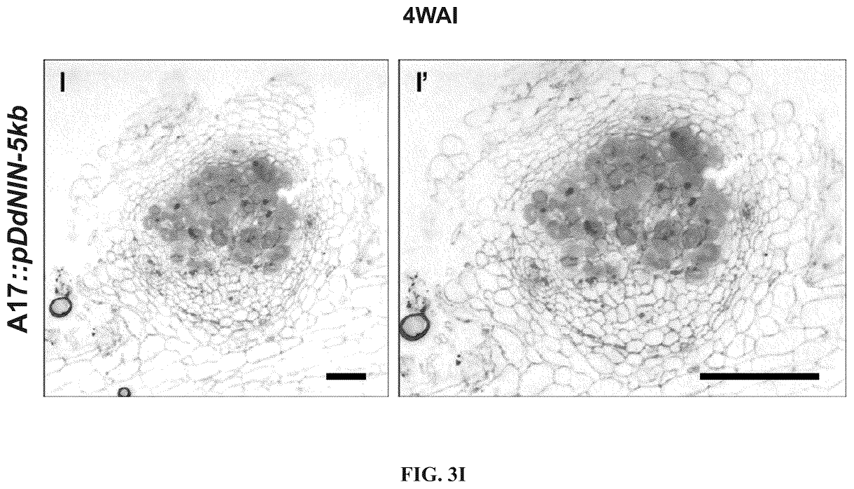

To check the spatiotemporal expression profile of the DgNIN during actinorhizal nodule development, the 5kb promoter pDgNIN_5kb was cloned upstream of the GUS reporter gene and transformed into the hairy roots of Datisca glomerata. The roots were harvested 8 weeks after infection (8WAI) with clade 2 Frankia Dg1. While the transformed roots under control condition did not produce any GUS staining, the primordia-like structure showed intense GUS stain throughout the structure (FIGS. 2A-2B). Comparatively, matured nodules only showed GUS staining at the upper part of the nodule with almost no staining in the proximal end (FIG. 2C). Ultrastructure analysis of the primordia reveals that the GUS staining was very prominent in the meristem, the vascular bundle, and the cortex, which is devoid of any identifiable infection zone (FIG. 2D). It is possible that this early induction of NIN was due to the presence of intercellular Frankia which were yet to form nitrogen fixing vesicles inside the plant cell. A similar early expression of Casuarina NIN in the infected root hairs indicated the role of actinorhizal NIN during infection (Clavijo et al., 2015). Matured nodules showed expression in the vascular bundle and meristem with the infected cells in the infection zone not showing much staining (FIG. 2E). The expression pattern was consistent with what had been observed previously in Casuarina where only uninfected cells adjacent to the infected cells showed pCgNIN:GUS expression (Clavijo et al., 2015). A duplication event in the Cucurbitales clade resulted in the formation of DgNIN2, and the promoter pDgNIN2_5kb showed some basal expression under control conditions, unlike pDgNIN_5kb (FIGS. 2F-2G). Although no primordia with GUS expression were detected, mature nodules did show staining, indicating GUS activity during nodule maturation (FIG. 2H). Ultrastructure analysis showed that, unlike pDgNIN 5kb, pDgNIN2_5kb was expressed near the infection zone and away from the meristem (FIGS. 2I-2J). The promoter activity was supported by the relative expression data in the D. glomerata nodules which shows ˜100 to ˜400-fold increase in expression for DgNIN and DgNIN2 respectively as compared to the roots (FIG. 1B). To check whether there is any conservation in the expression pattern of the NIN promoter between actinorhizal plants, the pDgNIN_Skb:GUS was transformed into the hairy root of M. truncatula A17 plants (FIGS. 3A-3I). As compared to the pMtNIN:GUS which was elicited at the base of the extending infection thread at 2WAI with Sinorhizobium meliloti Sm1021-eGFP, pDgNIN_Skb:GUS did not show any activation of GUS expression (FIGS. 3A-3B). In the later stage when the organogenesis was well established, however, the promoter activity looks comparable. pDgNIN_Skb:GUS showed very bright staining in the infection zone and vascular bundle (FIGS. 3B, 3C, 3E, and 3F).

To investigate whether actinorhizal NIN coming from another plant which was also infected by the clade 2 Frankia intercellularly behaved in a similar fashion, the 5kb region of the Dryas drummondii NIN was cloned upstream to GUS and transformed into hairy roots of D. glomerata. Eight weeks after infection there were primordia-like structures as well as matured nodules that showed GUS stain, as compared to no stain in the control transformed roots (FIGS. 2K-2L). Unlike the pDgNIN 5kb, the pDryasNIN_5kb showed staining in the distal part of the nodule away from the nodule meristem (FIG. 2M). Ultrastructure analysis further revealed that pDryasNIN_5kb was not active in the meristem of the primordia and was only present in the vascular bundle and cortex which was yet to form an infection zone (FIG. 2N). In the mature nodule the GUS staining was observed in the outer uninfected cortical layer and in the infected cells (FIG. 2O). Previous reports in M. truncatula indicated that NIN is expressed in the distal part of the nodule and specifically in the infection zone and the vascular bundle (J. Liu et al., 2021). Thus, the expression pattern of pDryasNIN is more resemblant of legumes as compared to pDatiscaNIN, supporting the inherent complexity of NIN regulation during nodule development. Expressing pDryasNIN_5kb:GUS in M. truncatula hairy root indicated their lack of epidermal expression post infection at two weeks after infection (FIG. 3G) as was previously observed in the case of pDgNIN:GUS. But by four weeks after infection, pDryasNIN_5kb showed comparable expression pattern to pMtNIN, which indicated that the organogenesis role of the promoter was conserved (FIGS. 3H-3I). The lack of epidermal role of NIN was recently reported for crack-entry legume Arachis hypogaea where the author claimed that NIN only has an organogenesis role (Bhattacharjee et al., 2022).

### Datisca NIN Promoters Respond to Exogenous LCO Treatment

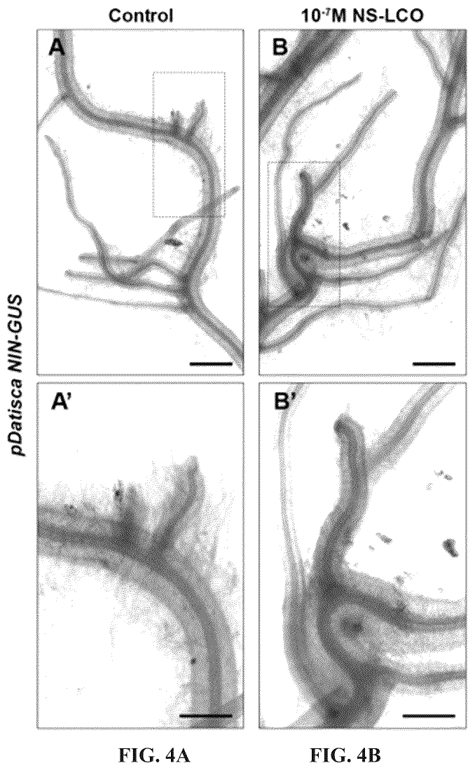

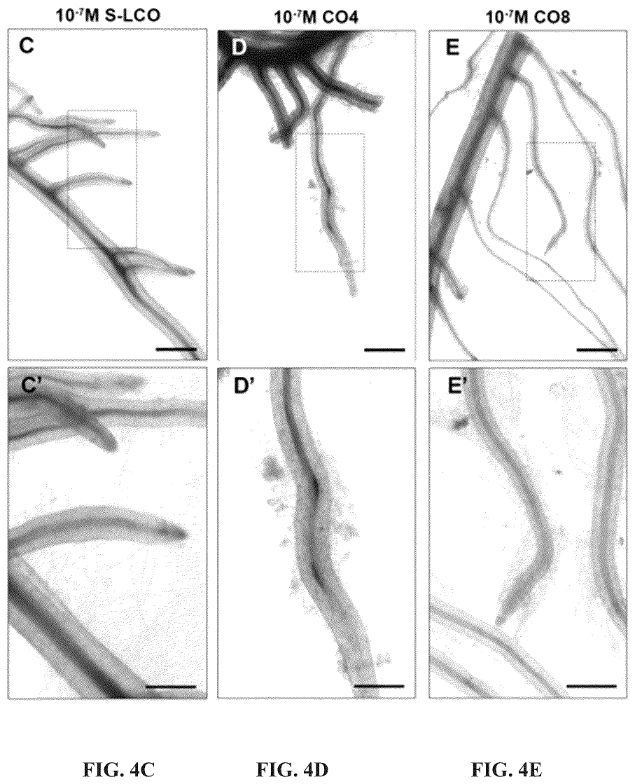

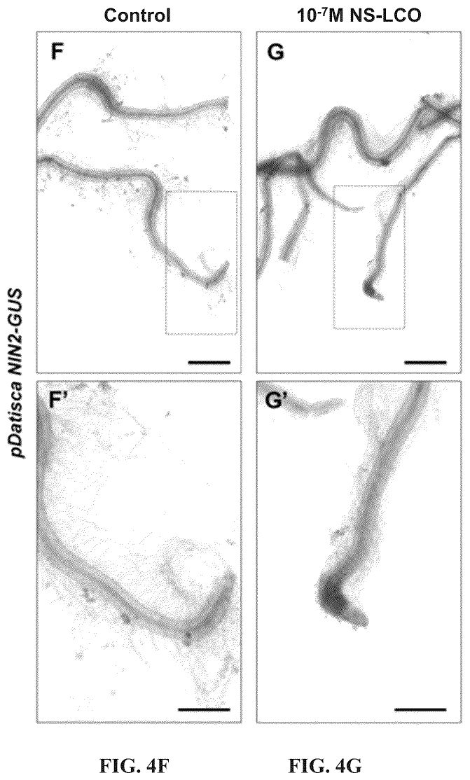

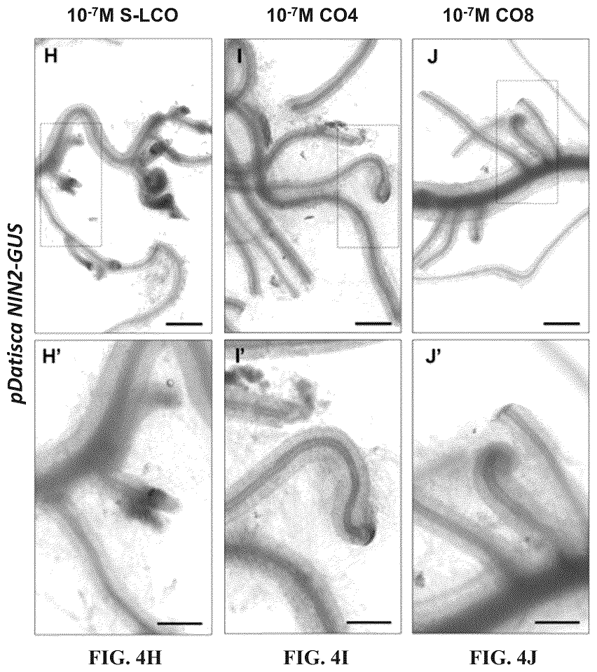

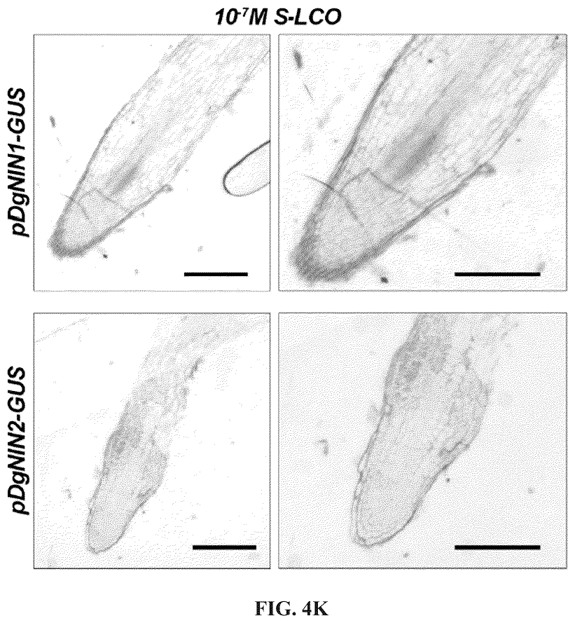

Clade 2 Frankia are the only group of Frankia that have retained the nod cassette and are capable of producing the chitin based signaling molecule (Nguyen et al., 2016). To investigate whether the D. glomerata responded to lipo-chitooligosaccharide (LCO) signaling molecules, hairy-root transformed plants carrying pDgNIN:GUS and pDgNIN2:GUS were transiently treated with LCO molecules. pDgNIN:GUS control roots treated with water did not show any staining (FIG. 4A). 24 hours post treatment with 10⁻⁷M S-LCO (sulphated-LCO) and 10⁻⁷M C04 roots showed intense staining in the root vasculature and near the root tip (FIGS. 4C-4D, 4K), as compared to no staining observed for 10⁻⁷M NS-LCO (nonsulphated-LCO) and 10⁻⁷M C08 (FIGS. 4B, 4E). pDgNIN2:GUS also did not show any staining under control treatment (FIG. 4F). As compared to pDgNIN:GUS, however, pDgNIN2:GUS did show staining in the 10⁻⁷M NS-LCO treatment (FIG. 4G) but did not show staining in the 10⁻⁷M C04 (FIG. 4I). The consistent staining in the 10⁻⁷M S-LCO indicated that the S-LCO was possibly a more preferred signaling molecule to trigger the NIN dependent symbiotic pathway in D. glomerata (FIGS. 4C, 4H). Surprisingly, however, the spatial distribution of the GUS staining for pDgNIN2:GUS was mostly in the root elongation zone with no staining in the vascular bundle (FIGS. 4G, 4H, 4K). This difference in expression profile for the two DgNINs was consistent to what was previously observed with Frankia (FIGS. 2A-2J). Similar to pDgNIN:GUS, 10⁻⁷M C08 failed to induce any NIN expression in plants with the pDgNIN2:GUS construct (FIG. 4J). Thus, it can be concluded that pDgNIN and pDgNIN2 were induced symbiotically both in the presence of Frankia and with the chitinous signaling molecule. More importantly, the duplication event led to neofunctionalization of the NINs, leading to different expression pattern in the paralogs.

### Downregulation of Datisca NIN Abolishes Nodule Development.

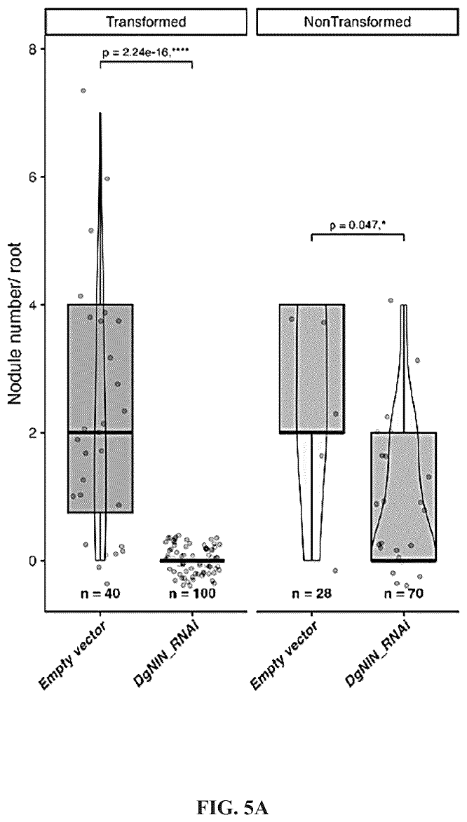

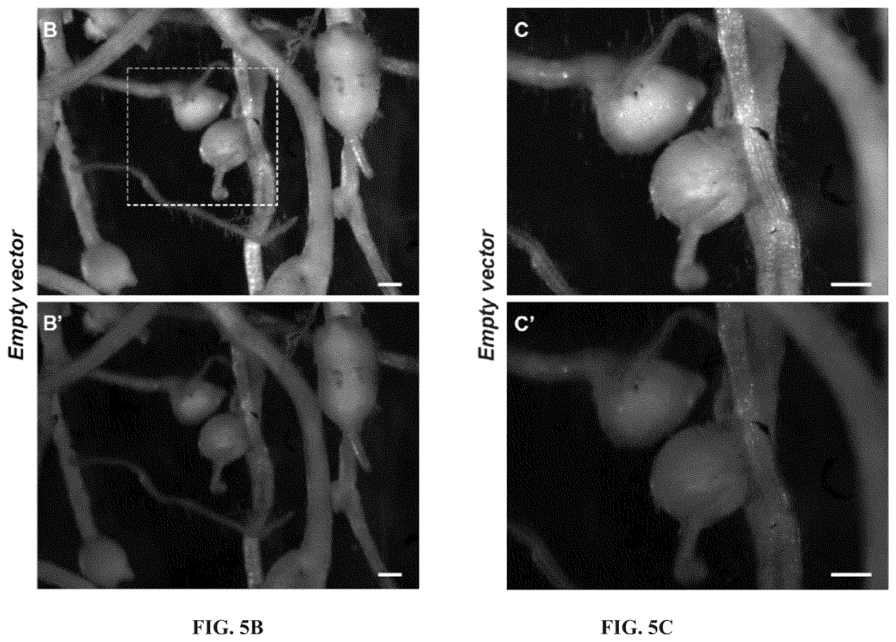

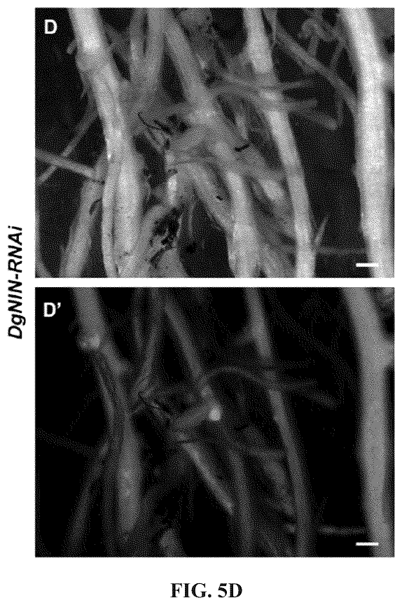

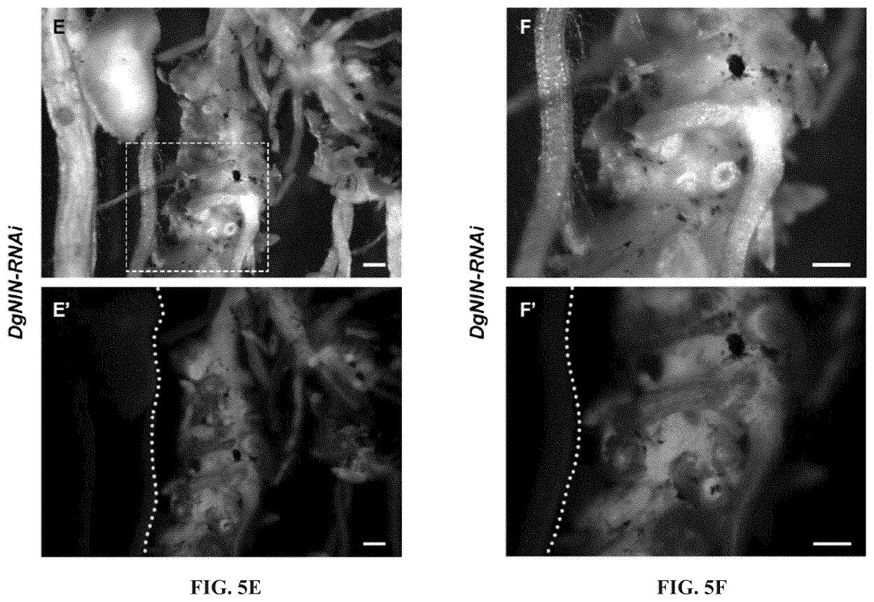

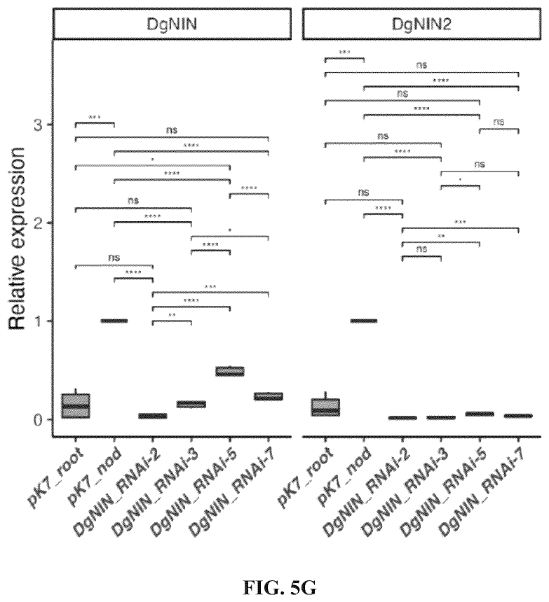

It is important to investigate the actual role of NIN during nodulation in D. glomerata, including establishing the spatiotemporal involvement of NIN during the intercellular actinorhizal nodule development. RNAi constructs targeting both DgNIN and DgNIN2 were designed and transformed into the hairy roots of D. glomerata eight weeks after infection with a cocktail of clade 2 Frankia Dg1 and Dg2. None of the transformed roots in DgNIN_RNAi plants (verified by the presence of the eGFP as the transformation marker) showed any formation of root nodule as compared to empty vector transformed hairy roots (FIGS. 5A-5D). While the empty vector transformed roots formed a median of 2 nodules per root (n=40), all the DgNIN_RNAi roots (n=100) were devoid of any visible nodule. It is important to note that the non-transformed roots in case of empty vector (n=28) and DgNIN_RNAi (n=70) formed nearly comparable and non-zero amounts of nodules per root (FIG. 5A). The significant reduction of nodule number in case of DgNIN_RNAi clearly indicates its importance in the nodule development process. At the same time, the formation of nodules in the non-transformed roots indicates the absence of any trans effect of the RNAi construct on the plant health. The importance of NIN in legume nodule development in well-established from the previous work in model legumes like M. truncatula and L. japonicus where the nin1 knockout mutant showed a strong nod-phenotype (Marsh et al., 2007; Schauser et al., 1999). In certain DgNIN_RNAi transformed roots (˜10%) there were some cluster root like structures present which were not noted in any of the empty vector transformed roots (FIGS. 5E-5F). The downregulation of the DgNIN and DgNIN2 were confirmed by real-time PCR against control transformed roots and nodules where the DgNIN_RNAi roots showed ˜90-100-fold reduction of DgNIN and DgNIN2 respectively in 3 out of the 4 independent root system tested. In soybean, RNAi of GmNIN1a and CRISPR of multiple NINs also resulted in complete abolishment of nodule development with severe reduction in expression level (Fu et al., 2022). Thus, it can be concluded that the lack of nodulation in DgNIN_RNAi transformed roots is directly related to the severe reduction in the expression level of NINs.

### Overexpression of NIN Abolished Nodulation and Resulted in Formation of Abnormal Structures.

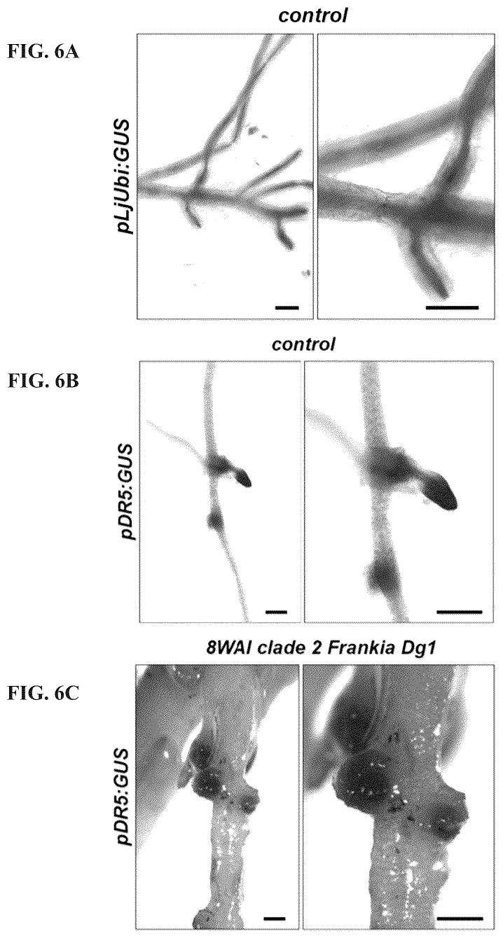

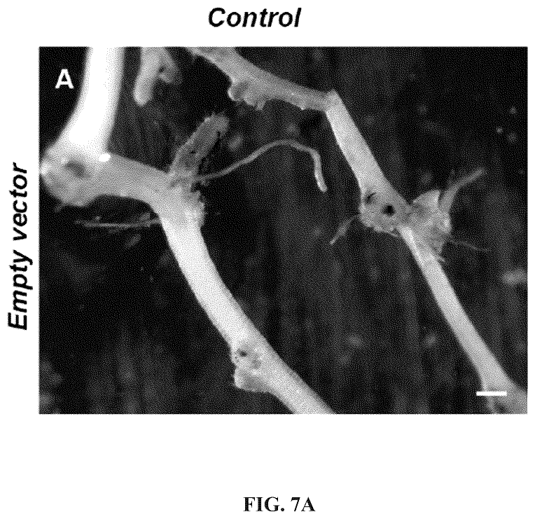

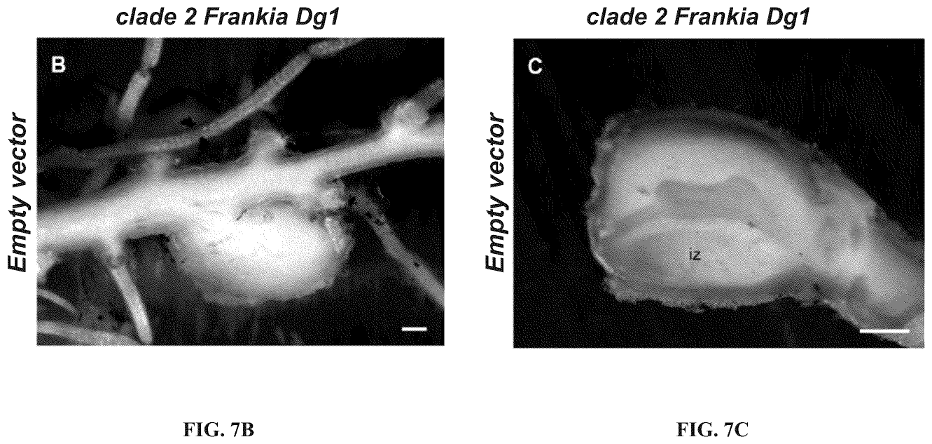

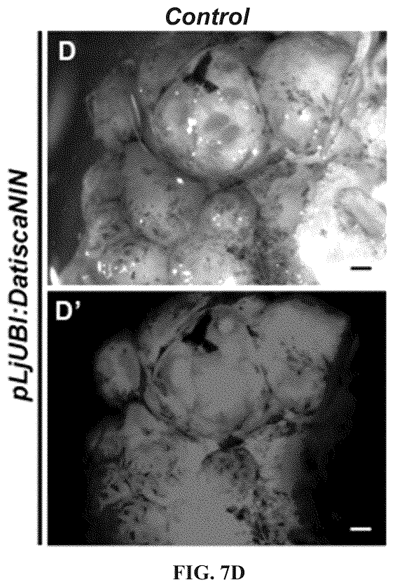

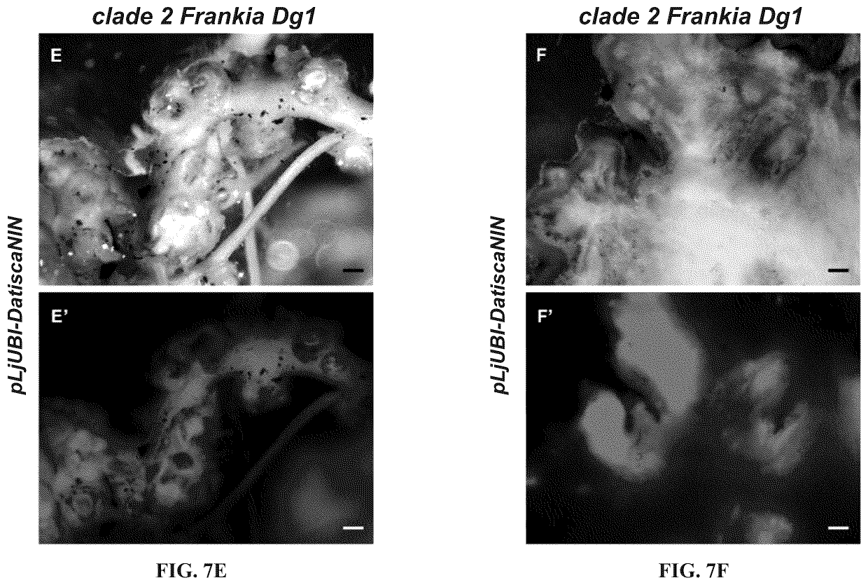

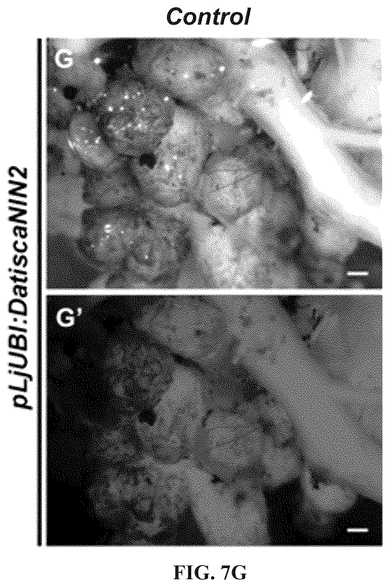

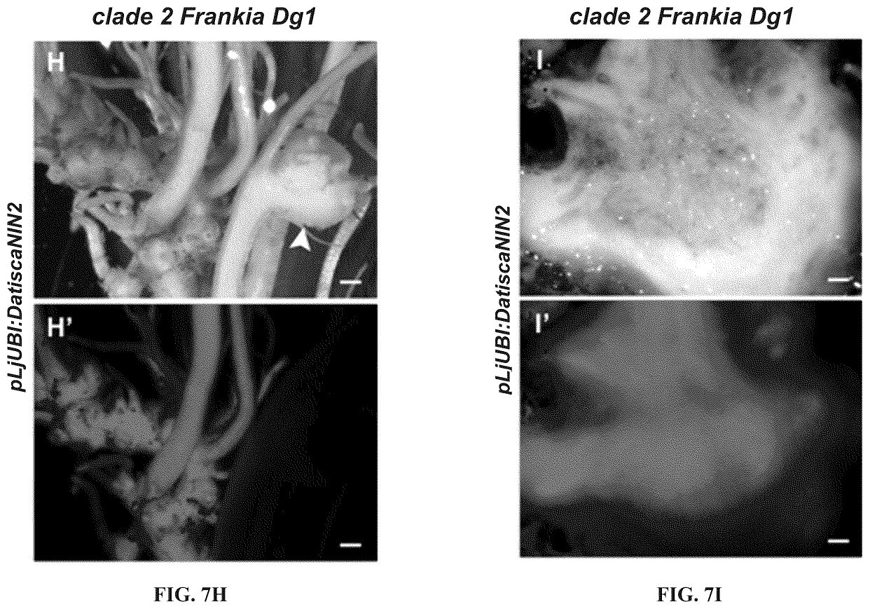

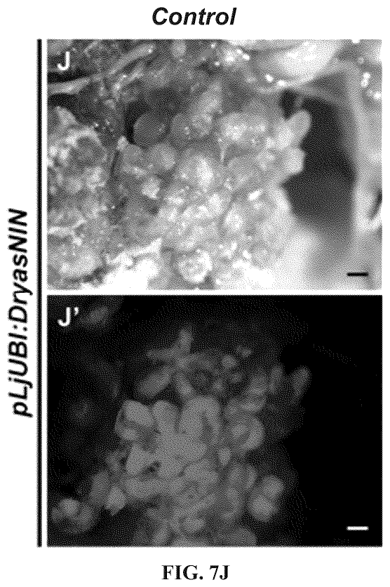

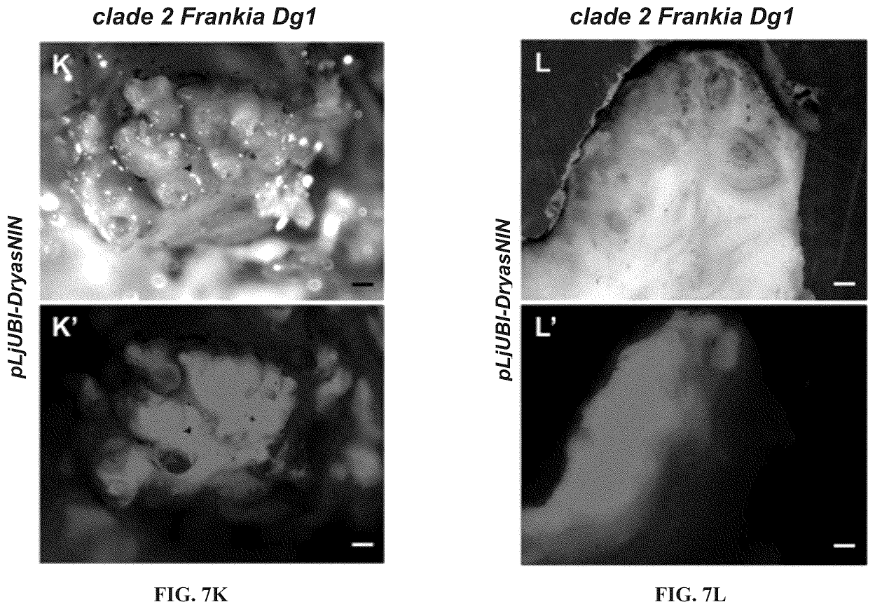

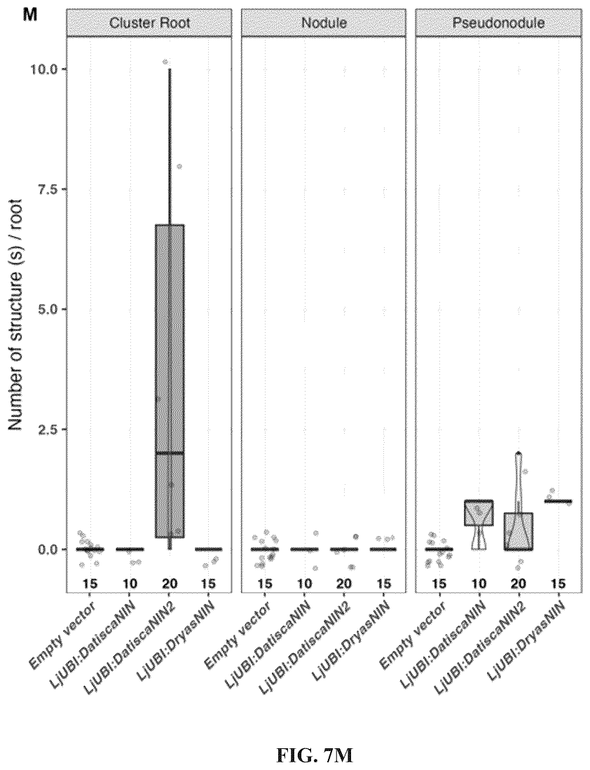

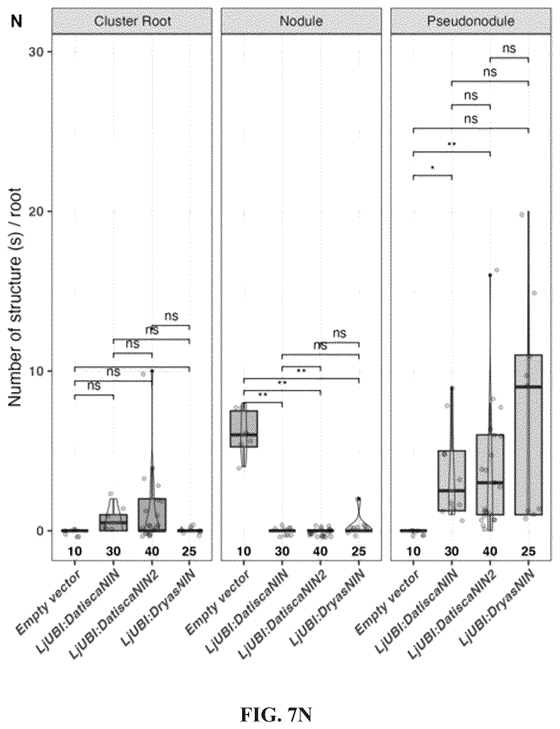

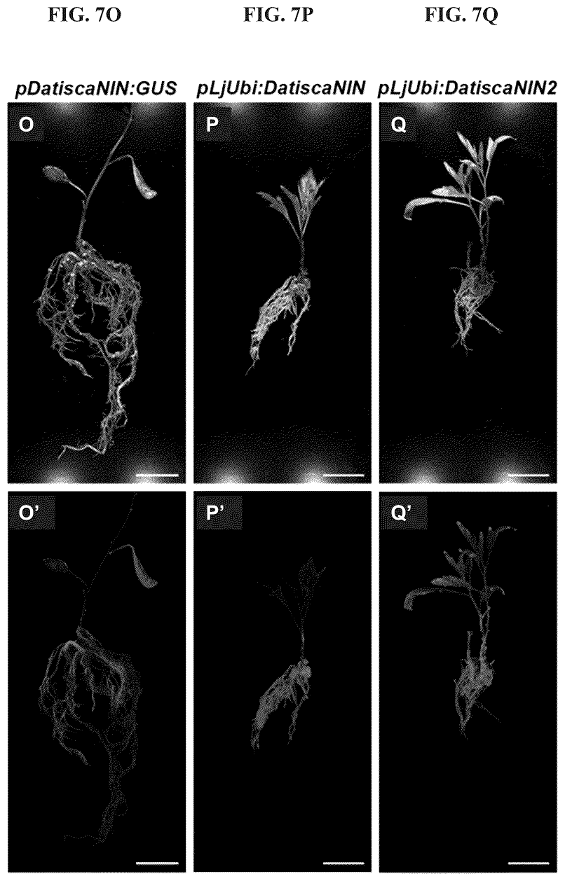

Previous work in model legumes showed that while overexpression of NIN can lead to spontaneous nodule organogenesis, it can also have a negative impact on infection, leading to empty nodule formation (Fu et al., 2022; Soyano et al., 2014). To check whether the impact of NIN overexpression on nodule development was also true for actinorhizal nodule organogenesis, DgNIN and DryasNIN were overexpressed under the pLotusUBI (pLjUBI) promoter. The pLjUBI promoter was checked for its activity in the Datisca hairy roots where it showed intense GUS staining, confirming its efficacy for overexpression experiments (FIG. 6A). The pLjUBI-DgNIN (FIGS. 7D-7F), pLjUBI-DgNIN2 (FIGS. 7G-7I), and pLjUBI-DryasNIN (FIGS. 7J-7L) were transformed into the hairy roots of D. glomerata and roots were scored 2 weeks after transfer to soil (2WTS) (control, FIGS. 7A, 7D, 7G, and 7J) and eight weeks after infection with clade 2 Frankia Dg1 (FIGS. 7B-7C, 7E-7F, 7H-7I, and 7K-7L). Overexpression of each of the DgNIN, DgNIN2, and DryasNIN resulted in formation of a huge callus like structure with many small bumps resembling either a trapped lateral root or an immature nodule primordium (FIGS. 7A, 7D, 7G, 7J, and 7O-7Q), as compared to normal root development observed for the empty vector construct (FIGS. 7M-7N). A minority (˜20%) of these plants formed non-transformed roots which were fundamental for their survival as the abnormal structures failed to produce healthy roots that could sustain the plant. Spontaneous nodule like structures were previously observed for NIN overexpression in legumes like M. truncatula, L. japonicus, and G. max (Fu et al., 2022; Soyano et al., 2013; Vernid et al., 2015). Unlike in legumes, however, where the overexpression resulted in formation of nodule like-structures, in D. glomerata the overexpression of actinorhizal NIN resulted in complete disruption of the normal root development.

The NIN overexpressing plants were transferred to soil and, following infection with clade 2 Frankia Dg1, roots were harvested eight weeks after infection (FIGS. 7B-7C, 7E-7F, 7H-7I, 7K-7L, and 7M-7N). While the non-transformed roots did produce nodules along with the control transformed roots (FIG. 7N), no proper nodule formation was detected on the transformed roots. Half-section of the structures showed the presence of significant amount of vascular proliferation and uninfected regions due to rapid cell division (FIGS. 7C, 7F, 7I, and 7L). In L. japonicus, overexpression of NIN suppressed nodule development by inducing the AON pathway (Soyano et al., 2014). Although there is no evidence of a functional AON pathway operating in actinorhizal plants, the lack of proper nodule development under NIN overexpression in D. glomerata suggested that it might be functional. From some of the callus-like structure in plants transformed with pLjUBI-DryasNIN, there was formation of extremely fat primordia-like structures on very small lateral roots (FIG. 7K). Very similar structures were also noted for GmNIN overexpression in soybean (Fu et al., 2022). On the other hand, half-section of the structure in pLjUBI-DryasNIN revealed the presence of several trapped primordia-like structures which did not manage to grow (FIG. 7L). As actinorhizal nodules are ontologically closer to lateral root development, these data indicated that the regulation of NIN expression is vital in controlling the homeostasis between lateral root development and nodule development.

### Actinorhizal NIN can Complement the Medicago Mutant Nin1.

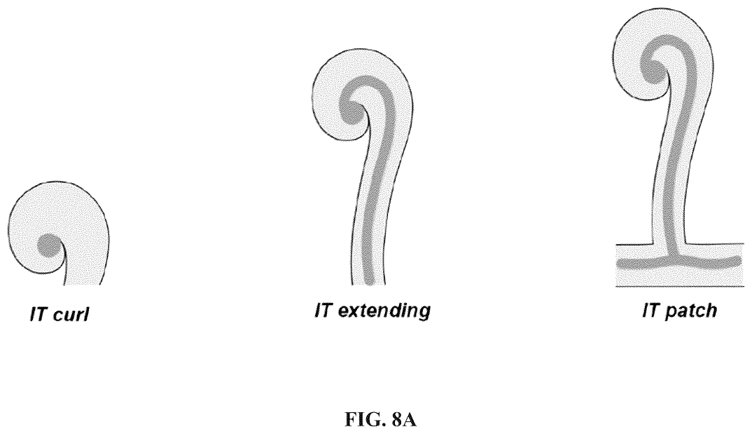

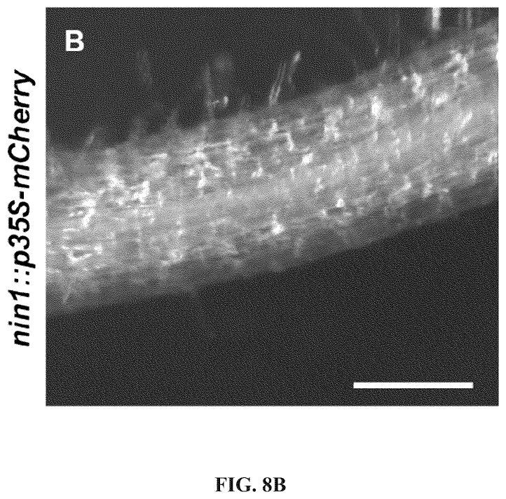

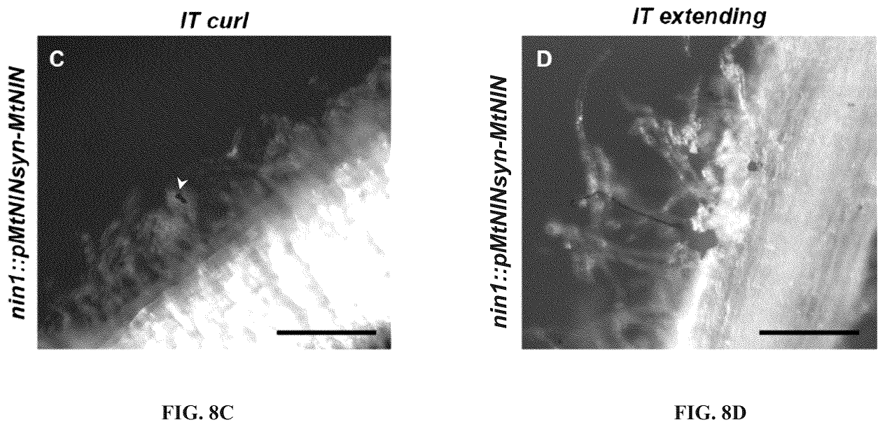

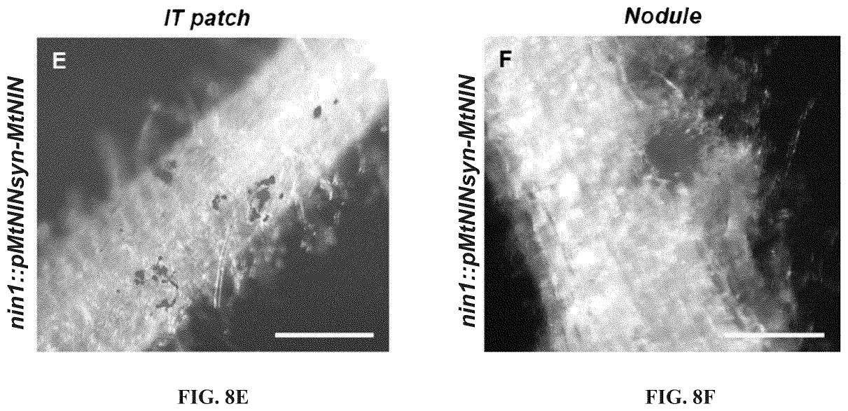

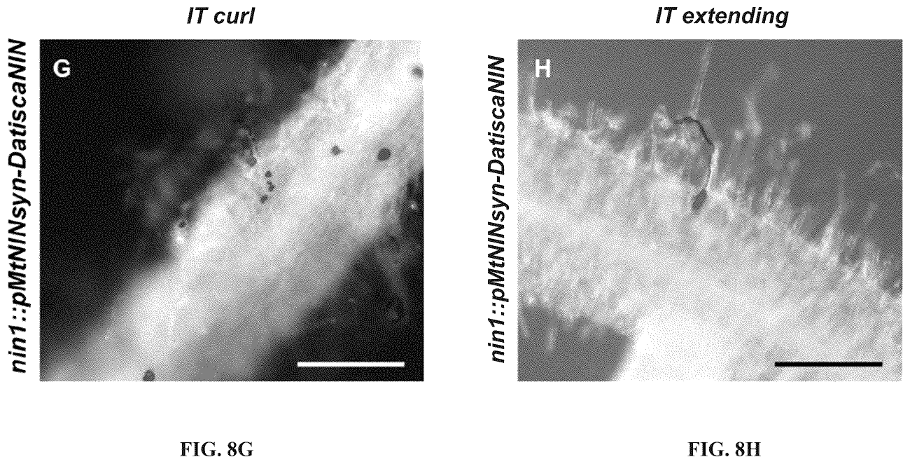

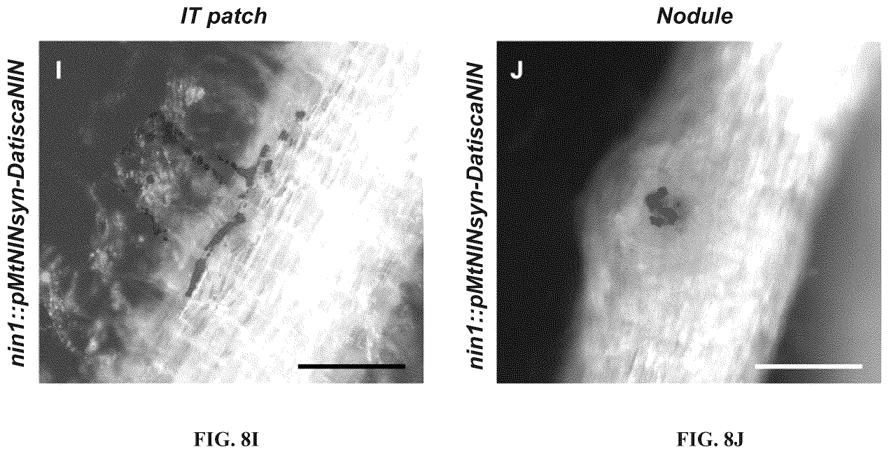

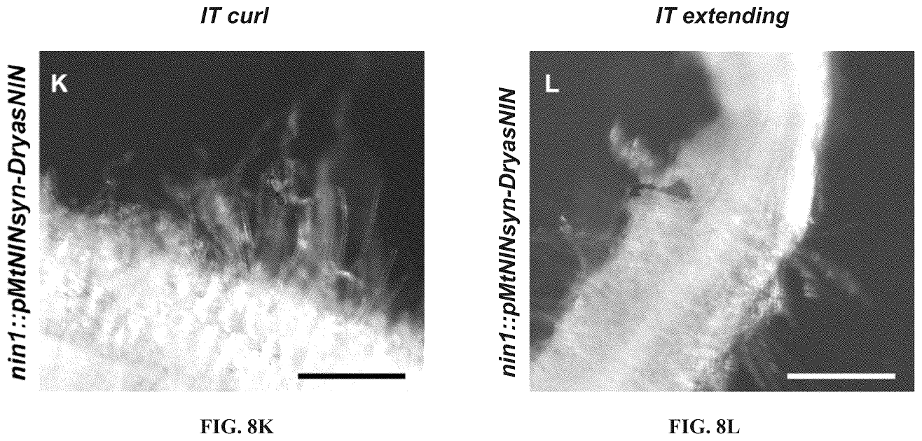

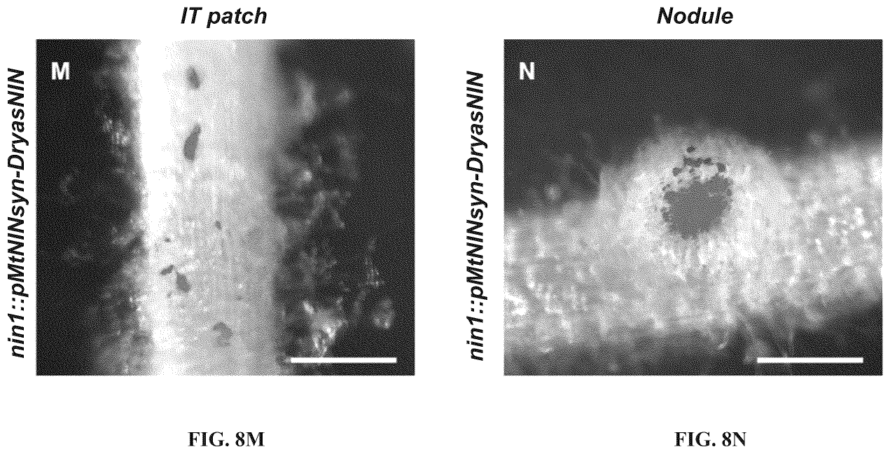

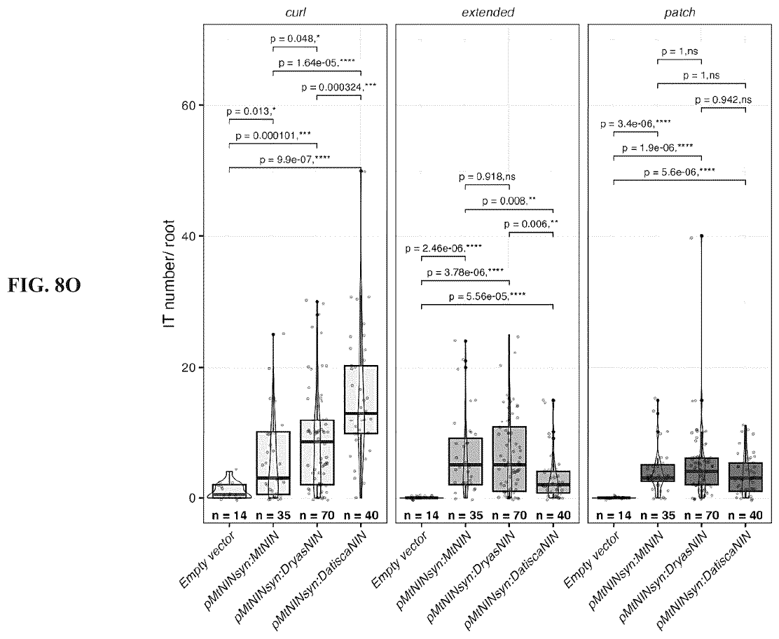

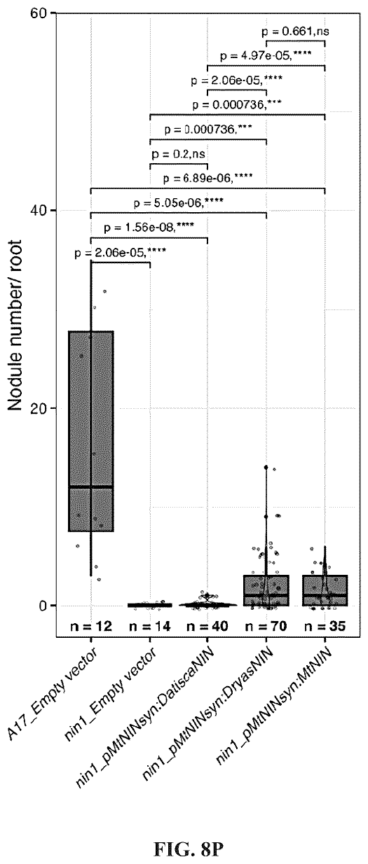

A cytokinin responsive element sitting several thousand base pairs upstream to the transcriptional start site (TSS) of the legume NIN was found to be indispensable for the rescue of the nin1 mutant in M. truncatula (J. Liu et al., 2019). To check whether there is cross-species conservation of the NIN protein function, rescue of the nin1 mutant was tested by hairy root transformation using the synthetic M. truncatula NIN promoter driving the DgNIN and DryasNIN. As a readout of the rescue efficiency, infection thread phenotype and the nodule organogenesis were scored (FIG. 8A). While the nin1 mutant transformed with the empty vector rarely showed formation of infection curl (˜5-7%), the pMtNINsyn:MtNIN transformed roots showed significantly higher numbers of infection curl, as well as extended infection threads that reached the epidermal layer and in some cases infection patches where the infection thread failed to extend beyond the epidermal layer (FIGS. 8B-8E and 8O). The pMtNINsyn:MtNIN transformed nin1 roots also managed to rescue nodulation but the number of nodules were significantly less than the A17:Empty vector (FIGS. 8F and 8P). The inability to match the A17 level of nodulation efficiency after rescue of nin1 mutant was previously reported during other experiments (Vernie et al., 2015). As compared to pMtNINsyn:MtNIN, pMtNINsyn:DgNIN produced significantly higher number of infection curls accompanied by lesser number of extended infection thread and infection patch (FIGS. 8G-8I and 8O). Concomitantly, the number of nodules formed on the transformed roots was also significantly less indicating the lesser efficiency of the D. glomerata NIN in rescuing nodule organogenesis (FIGS. 8J and 8P). On the other hand, pMtNINsyn:DryasNIN turned out to be almost equally efficient as the pMtNINsyn:MtNIN for the infection phenotype and during the nodule organogenesis (FIGS. 8K-8P). The efficiency of the Dryas NIN in rescuing the phenotype of the nin1 mutant indicated the functional conservation of the NIN protein. The ability of the actinorhizal NIN in the rescue of the nin1 mutant was demonstrated for the Casuarina NIN (Clavijo et al. 2015).

### Presence of Auxin Responsive Elements in the NIN Promoter Indicate its Ancestral Regulation.

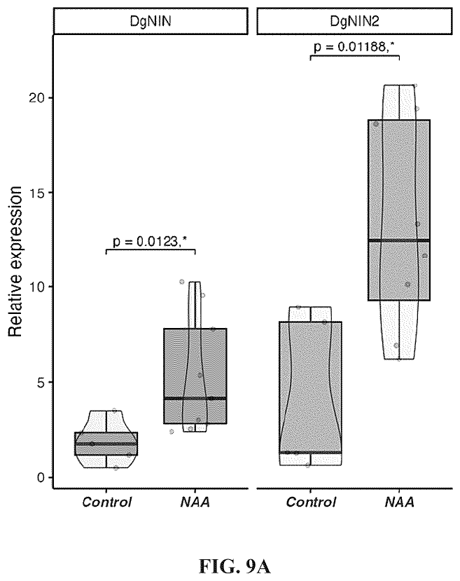

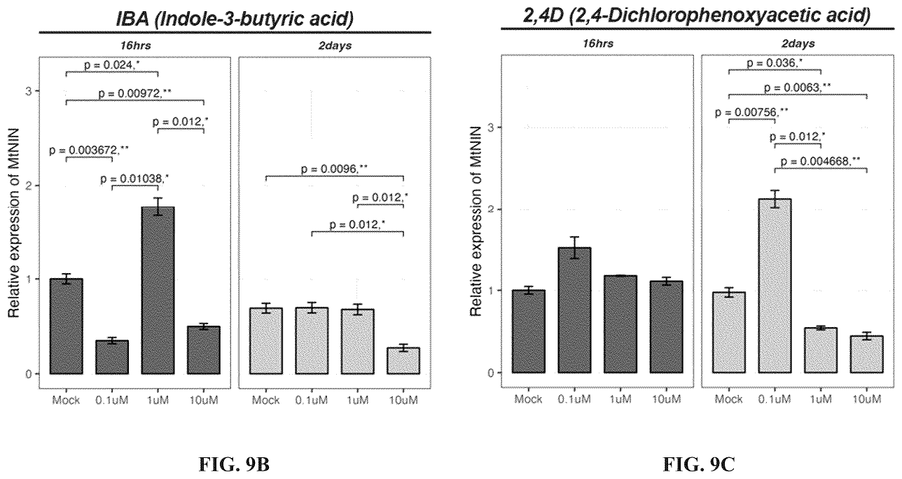

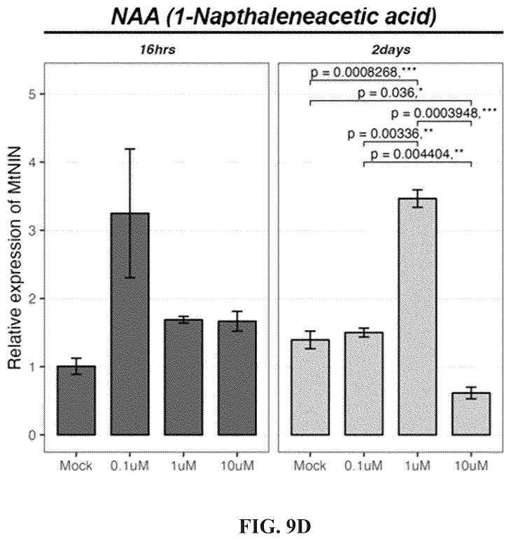

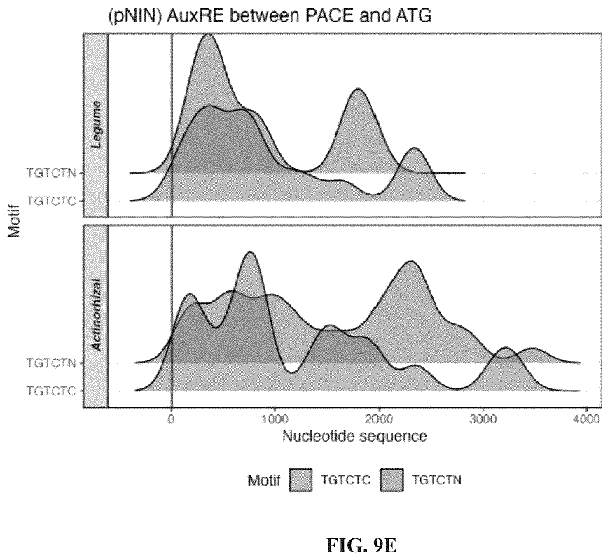

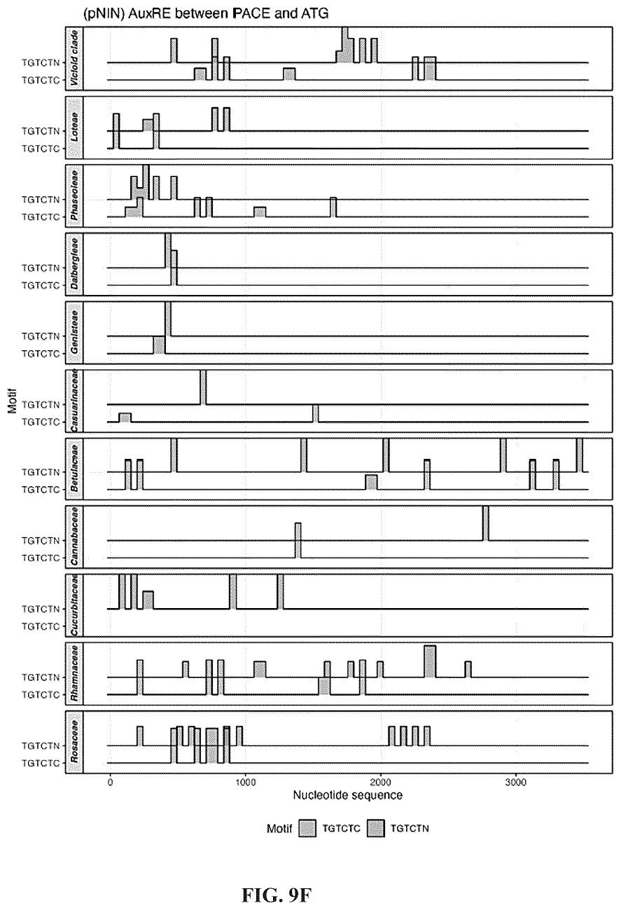

During nodule organogenesis, cytokinin is responsible for inducing the flavonoid biosynthesis pathway which act as a natural auxin transport inhibitor (Ng et al., 2015). The local increase in auxin concentration is vital for inducing de novo cell division and kick starting the organogenesis process in the pericycle and cortical cells (Kohlen et al., 2018a; Rightmyer & Long, 2011). Cytokinin is responsible for the induction of NIN through the cytokinin responsive elements in the promoter of NIN which are conserved in the legumes (J. Liu et al., 2019). Promoter analysis of the actinorhizal genomes failed to show the presence of any putative cytokinin elements homologous to the legumes. This is in concomitance with the previous report where the absence of any cytokinin element was confirmed in the Parasponia NIN promoter (J. Liu & Bisseling, 2020). As the actinorhizal nodule development shares homology with lateral root development where auxin plays an important role (Ibáñez et al., 2017; Pawlowski & Demchenko, 2012), the role of auxin in the expression of NIN in D. glomerata was investigated. 10 days post treatment (10 DPT) with 100⁻⁶M NAA (1-naphthalene acetic acid) a ˜4-fold and ˜12-fold increase in the expression of DgNIN and DgNIN2 was detected as compared to the control treated root samples (FIG. 9A). Similar exogenous treatment with auxin analogues such as IBA (Indole-3-butyric acid; FIG. 9B), 2,4D (2,4-Dichlorophenoxyacetic acid; FIG. 9C) and NAA (1-Napthaleneacetic acid; FIG. 9D) showed differential activation of MtNIN expression. M. truncatula treatment with 0.1μ M after 16 hours and 1 μM after 2 days resulted in ˜3-fold change in expression of MtNIN as compared to control treated roots (FIGS. 9B-9D). To understand whether the auxin dependent expression of NIN is conserved across the nodulating clades, the actinorhizal and legume NIN promoters were investigated to looked for putative auxin responsive elements (AuxREs). To do this analysis, the region between the Cyclops binding site (CycBS) and the transcriptional start site (TSS) was considered for all nodulating NINs (FIG. 9E). Previous study of AuxREs in the Arabidopsis genome indicated the prevalence of active AuxREs in the 5′UTR until −2kb upstream of the TSS (Mironova et al., 2014). Even though some of the CycBS in actinorhizal NIN promoter are further than −2kb, these were still included in the analysis (FIG. 9E). For the AuxRE motif search TGTCTC and TGTCTN (except C) were used, where the former is the canonical AuxRE and the latter is the motif which has been shown for ARF2/5 binding (Marchive et al., 2013; Ulmasov et al., 1997; Y. Zhang et al., 2015). A cumulative analysis of the legumes and the actinorhizal NIN promoter indicated the presence of a distinct peak closer to the CycBS and another peak closer to the TSS which was more prominent in legume and much more diffused for the actinorhizal plants (FIG. 9E). Among the actinorhizal clade Rosales which include Rosaceae plant D. drummondii and Cercocarpus sp. and Rhamnaceae plants like Ceanothus and Ochetophila showed more putative AuxRE as compared to other intercellular actinorhizal clade of Cucurbitales (FIG. 9F). Among the Fagales, Alnus from Betulaceae showed more putative AuxREs as compared to Casuarina from Casuarinaceae. Within Fabales, the Viciod clade which include M. truncatula and C. arietinum, Lotea represented by L. japonicus, and Phaseoleae represented by Vigna, Cajanus and Glycine showed more putative AuxREs compared to Arachis (Dalbergeae) and Lupin (Genisteae) (FIG. 9F). Parasponia also showed very few AuxREs, indicating that intercellular rhizobial entry nodulating plants showed a smaller number of the AuxREs motifs in the NIN promoter.

### Synthetic Auxin Responsive Promoter-Driven NIN can Induce Spontaneous Nodule-Like Structures.

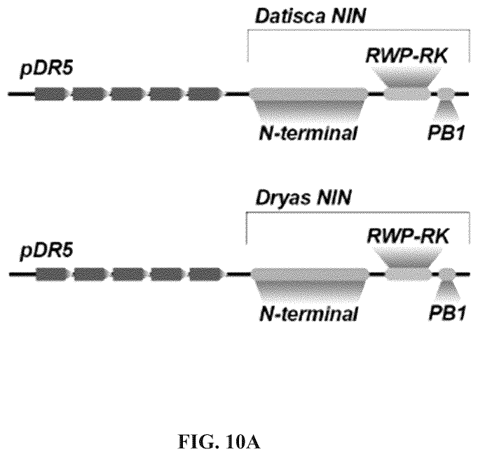

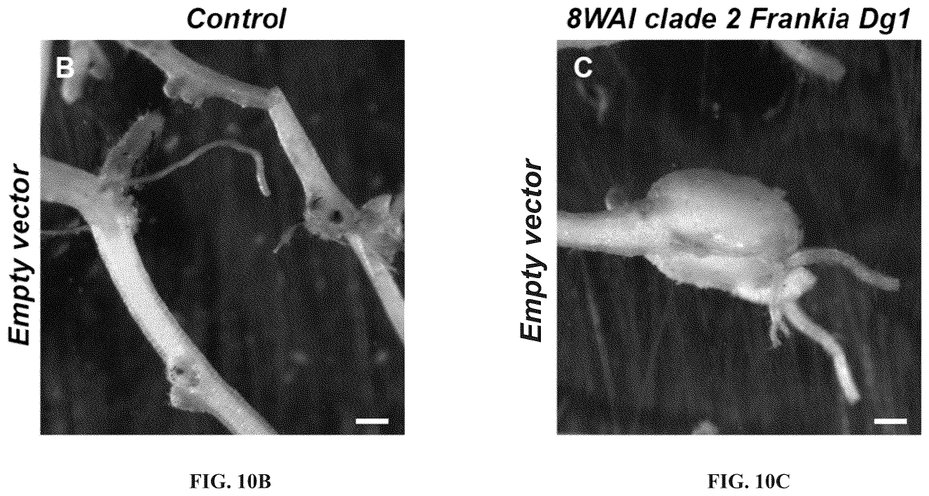

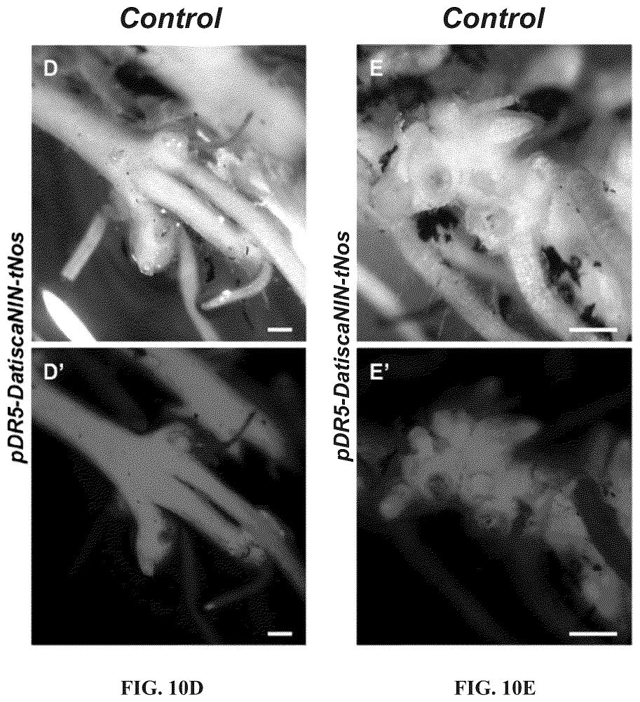

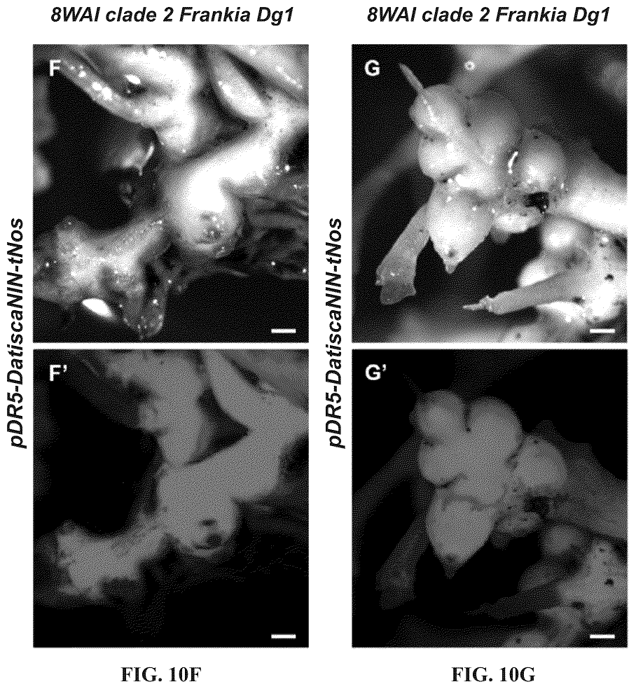

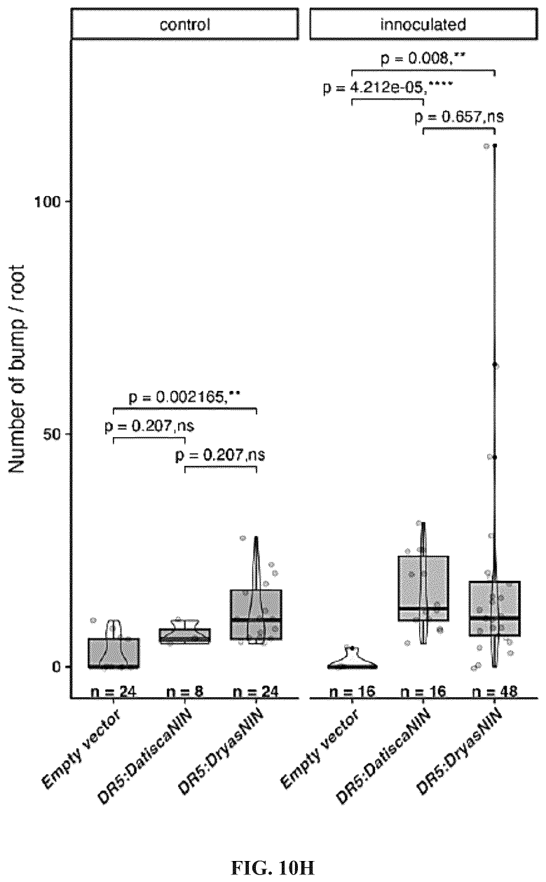

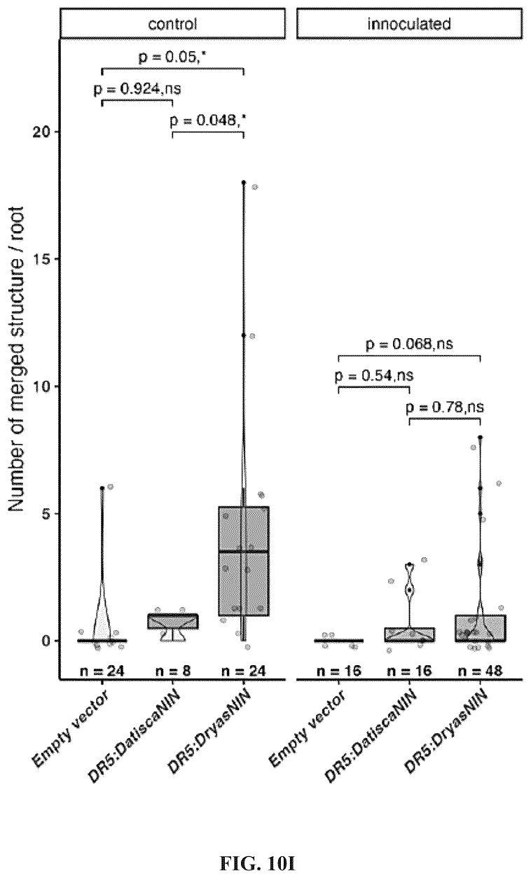

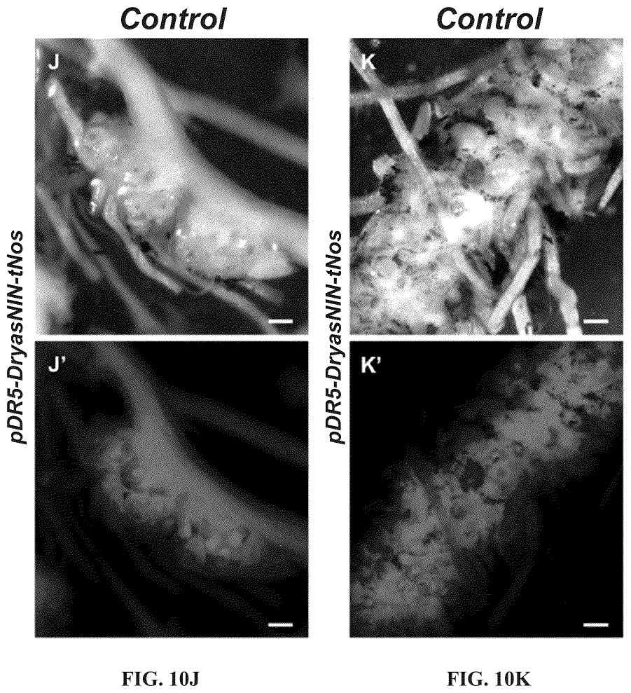

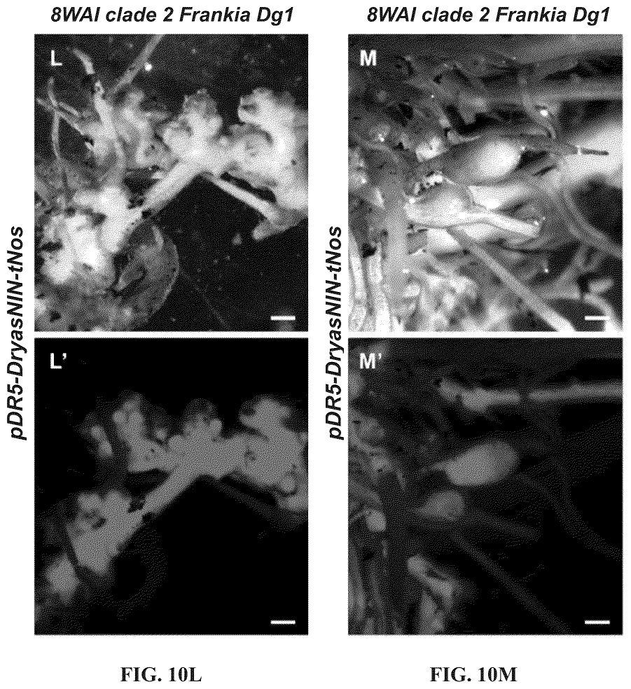

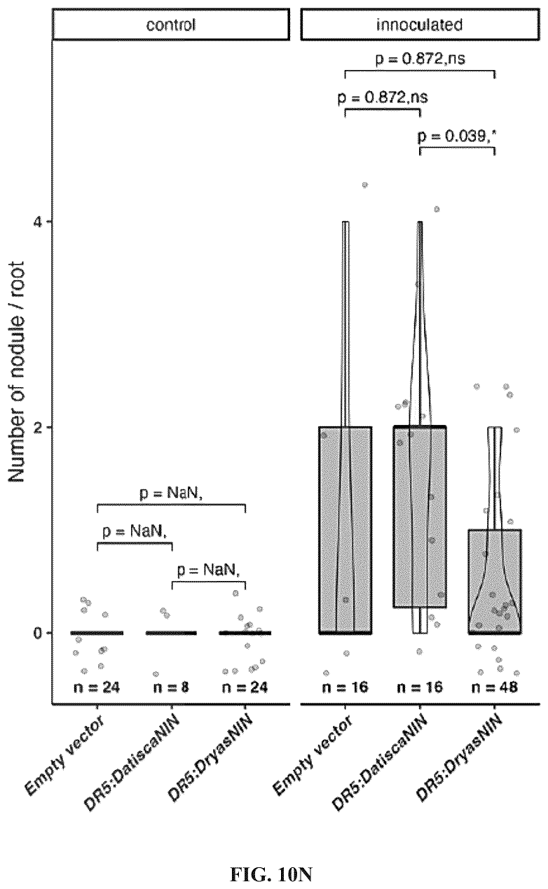

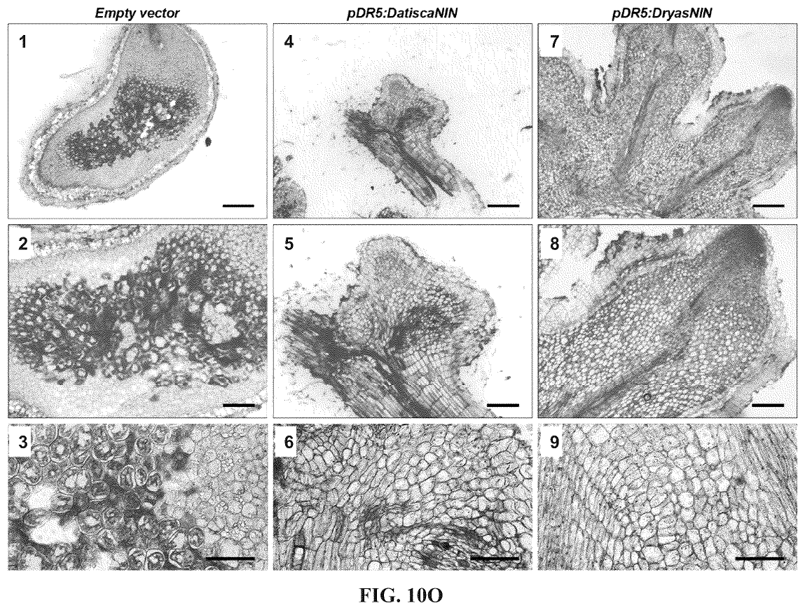

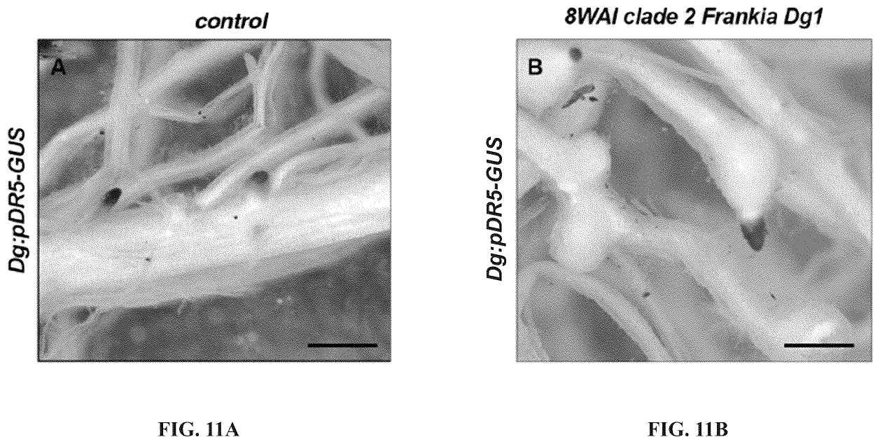

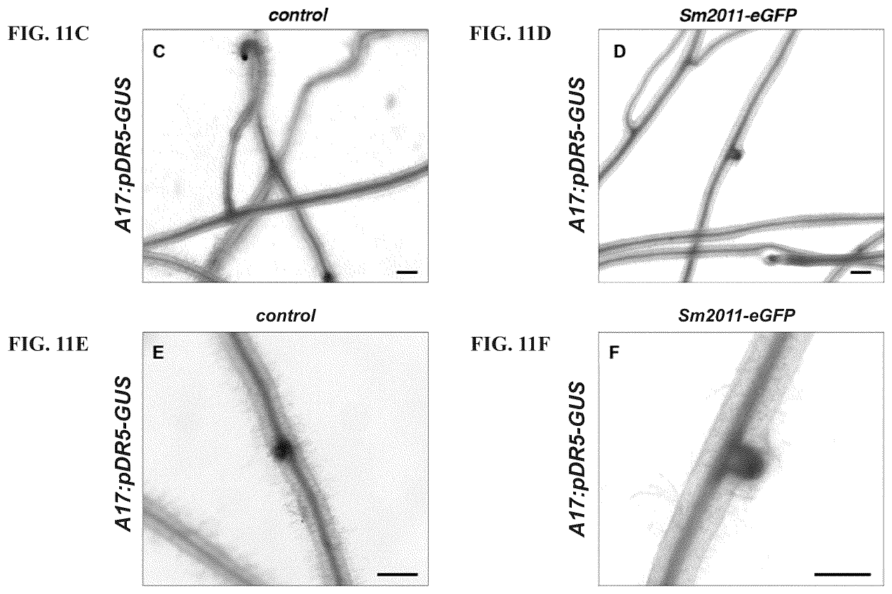

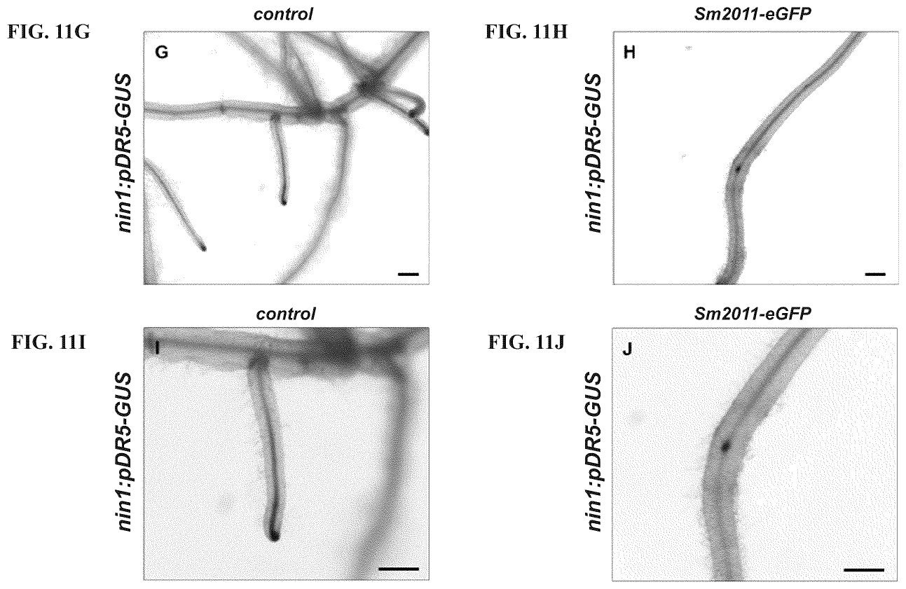

The presence of the auxin responsive hexameric motif is not sufficient to determine its efficacy during the transcriptional process, and analysis in Arabidopsis showed that only 50% of the predicted genes responded to exogenous auxin treatment (Freire-Rios et al., 2020). To check the role of auxin in NIN activation during actinorhizal nodule development, DgNIN and DryasNIN were ectopically expressed using the synthetic auxin responsive promoter pDR5 in the hairy roots of D. glomerata (FIG. 10A). pDR5:GUS is active during the lateral root development in D. glomerata and also during nodule development (FIGS. 11A-11B). The pDR5:GUS activity could only be detected at the nodule meristem, however (FIG. 11B), reconfirming what was previously published for D. glomerata (Demina et al., 2019). Unlike overexpression of NIN which resulted in formation of a callus like-structure (FIGS. 7A, 7D, 7G, and 7J), pDR5:DgNIN and pDR5:DryasNIN resulted in formation of spontaneous nodule like-structures referred to herein as bumps (FIGS. 10D, 10H, and 10J). While under control condition the number of bumps in plants with pDR5:DryasNIN was significantly higher than plants that received the empty vector control, post infection with clade 2 Frankia Dg1 plants expressing NIN from either construct had significantly higher number of bump formation than plants that received the empty vector control (FIGS. 10F, 10H, and 10L). This is in concert with the fact that Frankia produce auxin during the infection process which could affect the sensitivity of the DR5 promoter driving NIN (Hocher et. al., 2011). Apart from the bump, there was also formation of cluster root like-structures in pDR5:DryasNIN where the lateral roots cluster together with complete loss of spatial distribution (FIGS. 101 and 10K). While such cluster root also formed on the Empty vector and pDR5:DgNIN roots under control condition, the number of such structure was significantly higher in pDR5:DryasNIN (FIGS. 10E, 10I, 10K, and 10L). Post infection the prevalence of cluster root formation was very low in pDR5:NIN transformed roots, and proper healthy nodule formation was visible in the adjoining roots (FIGS. 10G, 10M, and 10N). Previously, cluster root formation was observed in Lupin under phosphate (P) starved conditions as an adaption to acquire nutrients (Wang et al., 2013). Although cluster roots were never previously observed in D. glomerata, it could be possible that the influence of pDR5:NIN coupled with nutrient-deprived condition induced such structures. This is in concomitance with the fact that cluster root formation can be induced under the influence of auxin (Gilbert et al., 2000; Skene, 2001). Thus, this result indicated that during intercellular actinorhizal nodule development in D. glomerata, auxin orchestrated the expression of NIN to drive the organogenesis program.

Synthetic Auxin Responsive Promoter-Driven NIN Results in More Infection Events in M. truncatula.

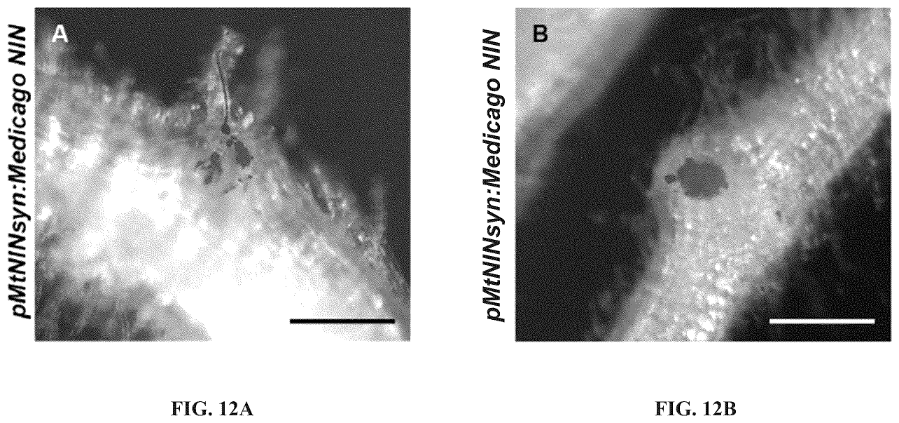

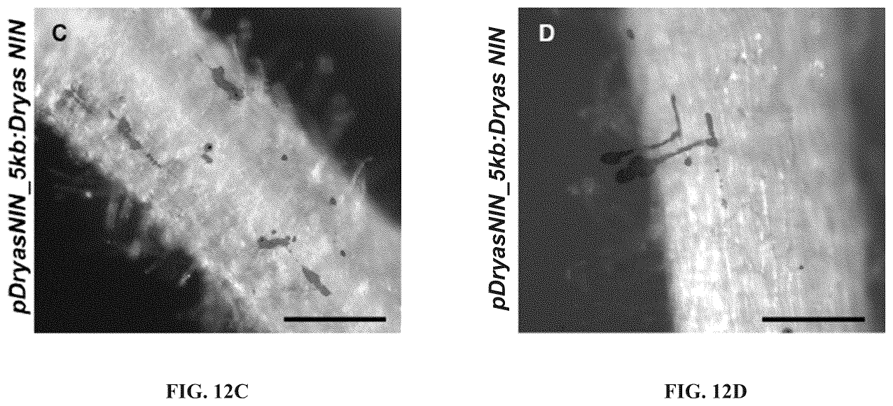

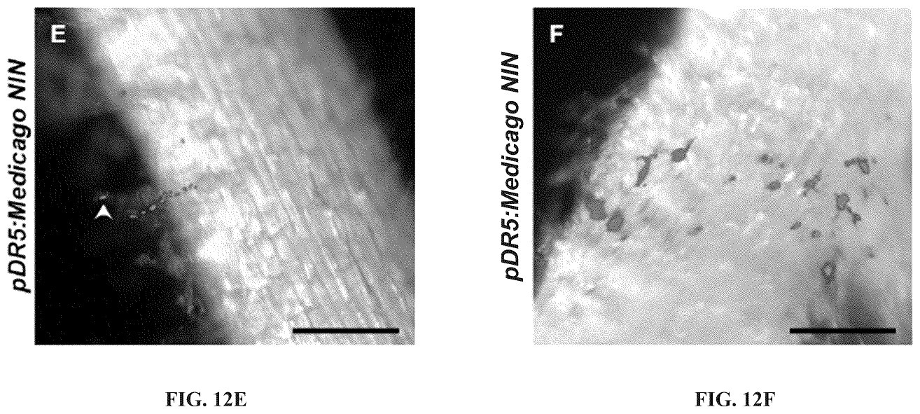

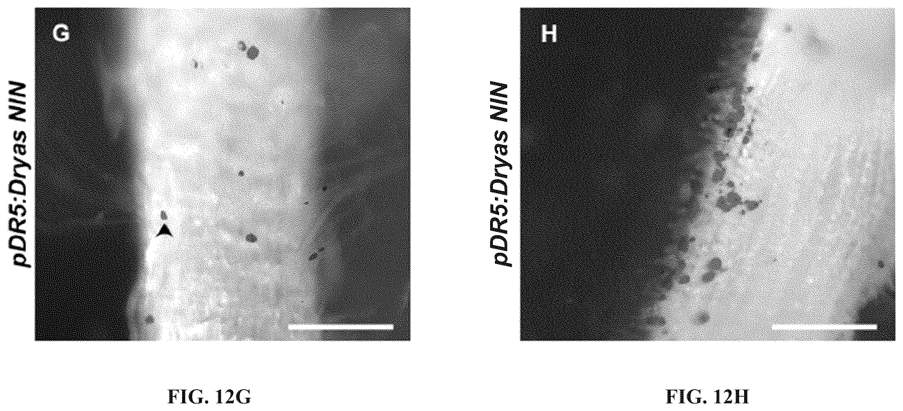

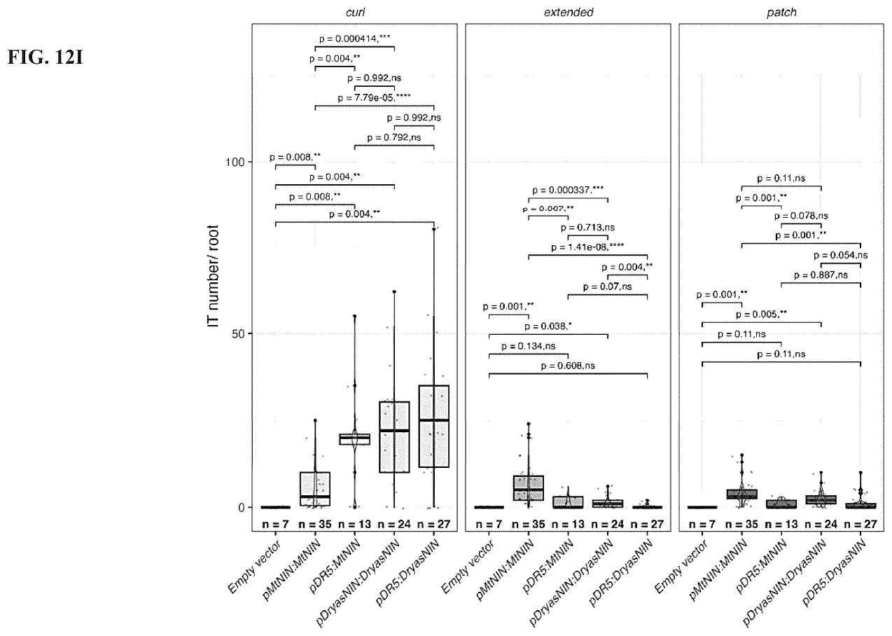

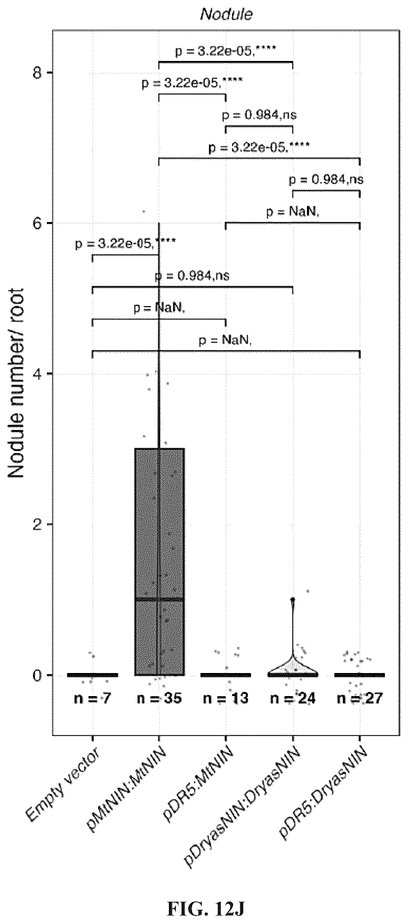

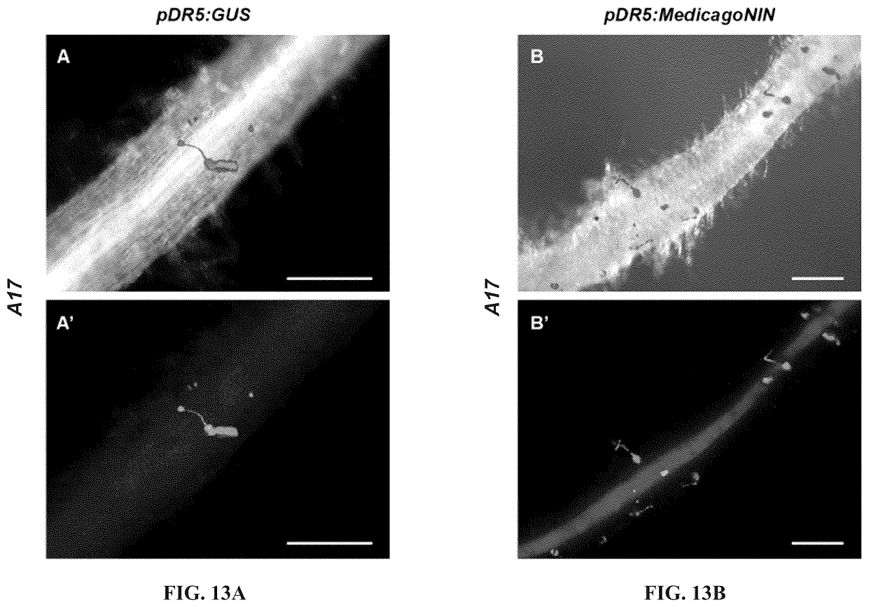

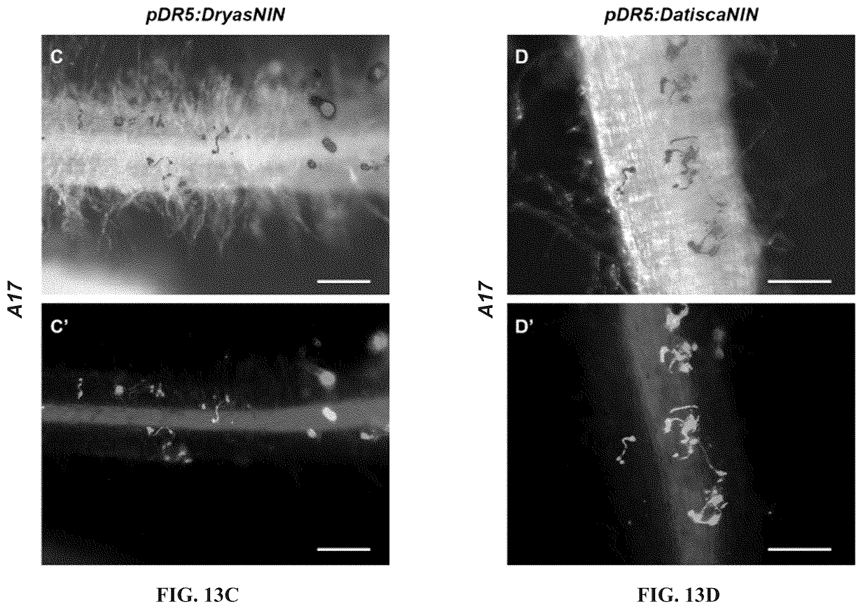

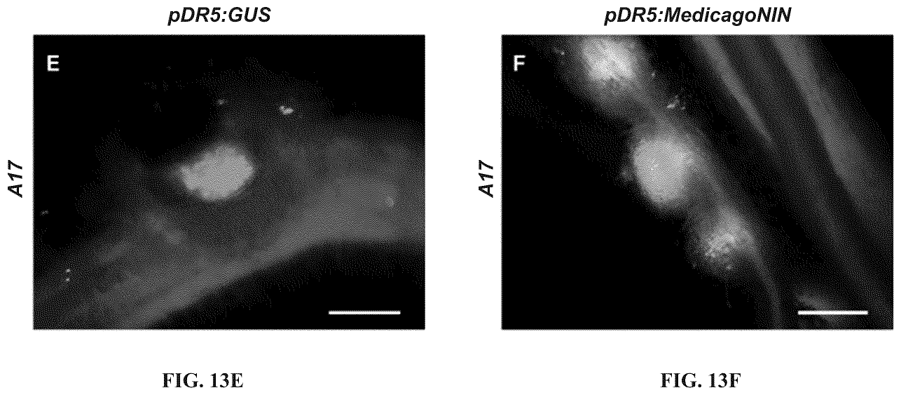

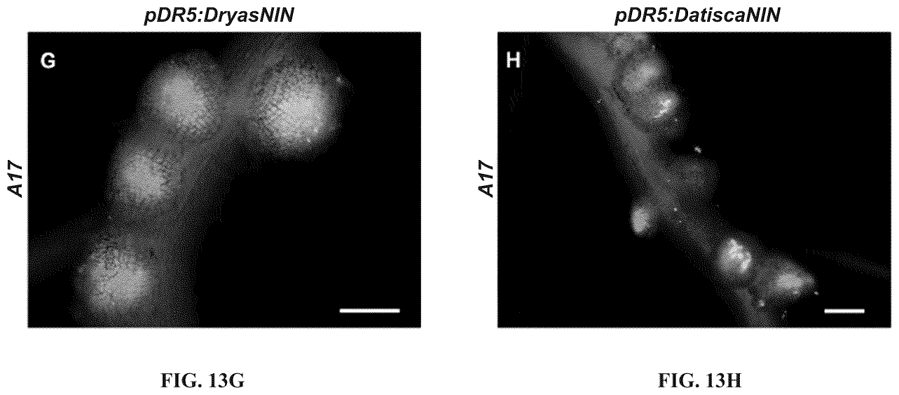

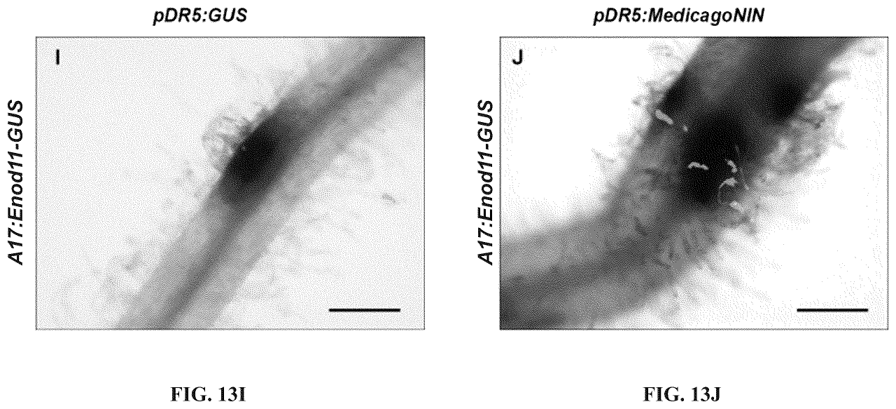

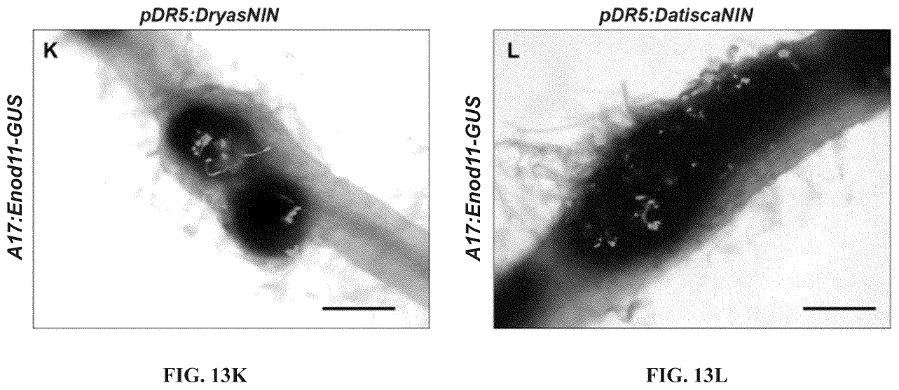

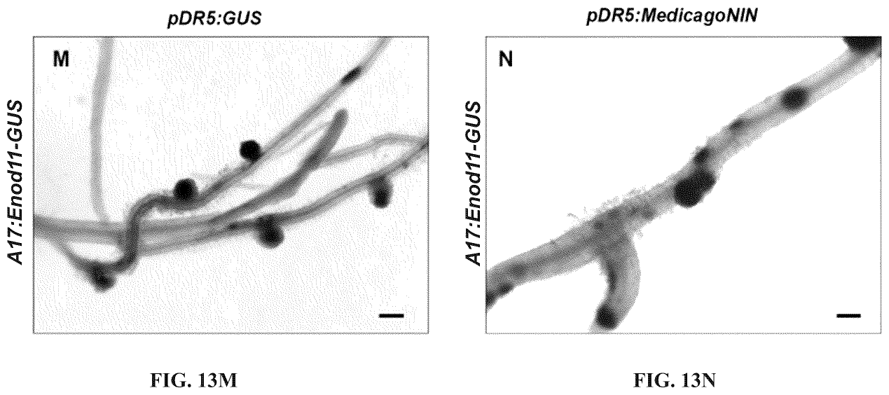

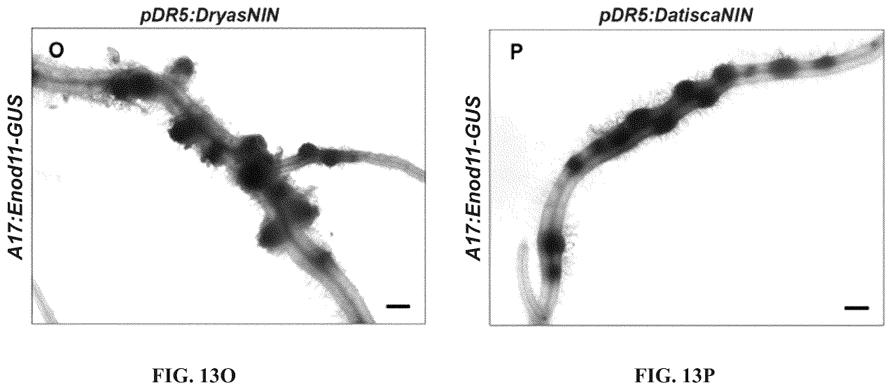

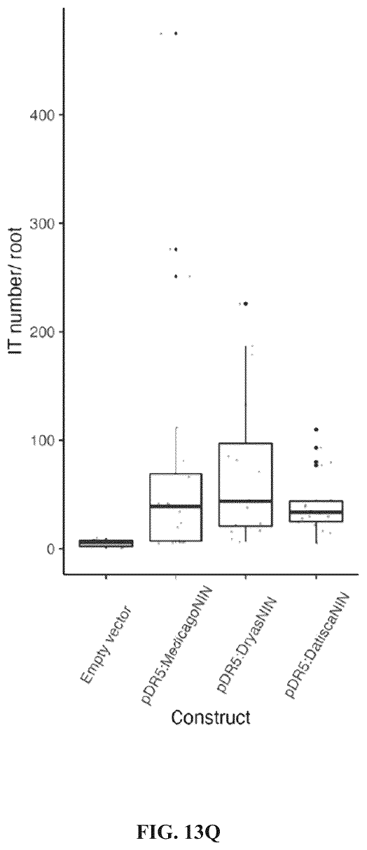

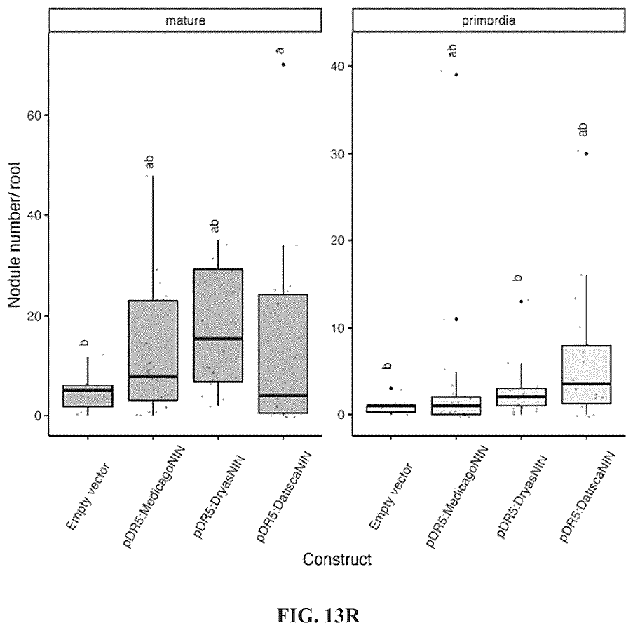

Plants need to alter their gene expression profile to respond to changing environmental cues. Previous evidence in salt stress tolerance shows that plants prefer to have conserved trans-regulators but evolutionarily divergent cis-regulatory sequences (Wu et al., 2021). In order to find whether the AuxREs that are present in the NIN promoter are evolutionarily conserved in controlling NIN expression, pDR5:NIN from M. truncatula and D. drummondii were introduced into the hairy-roots of the M. truncatula nin1 mutant. As a control, the hairy-roots were also transformed with pMtNINsyn:MtNIN and pDryasNIN 5kb:DryasNIN (FIGS. 12A-12J). 4WAI with S. meliloti 2011-eGFP, the pMtNINsyn:MtNIN formed nodules that were evident from the eGFP fluorescence driven by an nifH promoter (FIGS. 12A-12B), but none of the other constructs produced any nodules (FIGS. 12C-12J). pDryasNIN_Skb:DryasNIN also could not to rescue the nodulation in the nin1 mutant, with nodules forming in rare cases (FIGS. 12C, 12D, and 12J). Although previously identical expression patterns were visible for the pDryasNIN_5kb and pMtNINsyn constructs (FIGS. 3A-3I), the inability of the pDryasNIN promoter to rescue nodulation indicated the importance of its missing cytokinin responsive elements. However, the hairy roots did show the presence of infection threads that managed to reach the epidermal layer, with some even forming infection patches (FIGS. 12C-12D). The ability of the pDryasNIN 5kb:DryasNIN to induce epidermal infection threads is consistent with what had previously been observed for pMtNIN_Skb:MtNIN where the CycBE (Cyclops Binding element) was responsible for the epidermal infection thread progression (J. Liu et al., 2019). On the other hand, the pDR5:MtNIN and pDR5:DryasNIN both resulted in formation of significantly higher numbers of infection curls which rarely managed to reach the epidermal layer (FIGS. 12E-12I). Unlike in D. glomerata where the pDR5:DryasNIN and pDR5:DatiscaNIN resulted in spontaneous nodule like structure formation (FIGS. 10D-10E and 10J-10K), no nodule-like structure formation was detected in M. truncatula. To investigate the possible reason behind the discrepancies in the phenotypic effect, the expression level of pDR5 was assayed in the A17 wild-type background and in the nin1 mutant background (FIGS. 11C-11J). Under control conditions pDR5:GUS was active throughout the root system in both A17 and the nin1 mutant, with significantly higher expression in the root tips (FIGS. 11C, 11E, 11G, and 11I). But post infection, the pDR5:GUS expression level in the nin1 mutant was significantly less in than the A17 wild-type, where the auxin signaling is active both in the vascular bundle as well as the nodule meristem (FIGS. 11D, 11F, 11H, and 11J). This agrees with the previous observation that post infection the NIN is responsible for auxin production by activating auxin biosynthesis genes like LBD16 and YUCCA (Schiessl et al., 2019; Shrestha et al., 2021). As the A17 background provided ample auxin induction, the introduction of the pDR5:MtNIN, pDR5:DryasNIN, and pDR5:DatiscaNIN all resulted in the formation of significantly higher numbers of nodules developing possibly due to significantly higher number of infection events by eight weeks after infection (FIGS. 13A-13H, 13Q, and 13R). This was confirmed in the A17:Enod11-GUS background as well where infection patches with higher number of infection threads were seen (FIGS. 13I-13P). Thus, AuxREs are active in the NIN promoter but, unlike in the D. glomerata actinorhizal system where the AuxRE is controlling the organogenesis pathway, in M. truncatula its role is primarily to control the infection process.

### The Auxin Responsive Element in the Medicago NIN Promoter is Essential for Infection Progression.

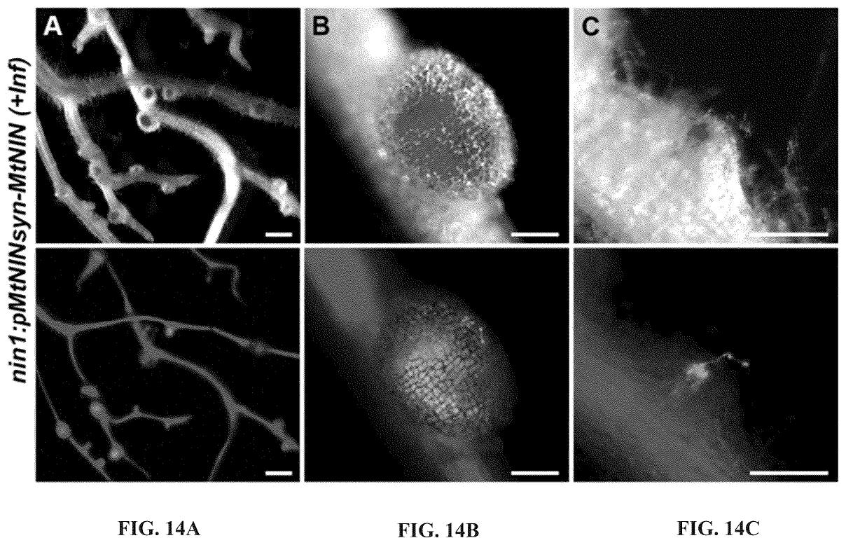

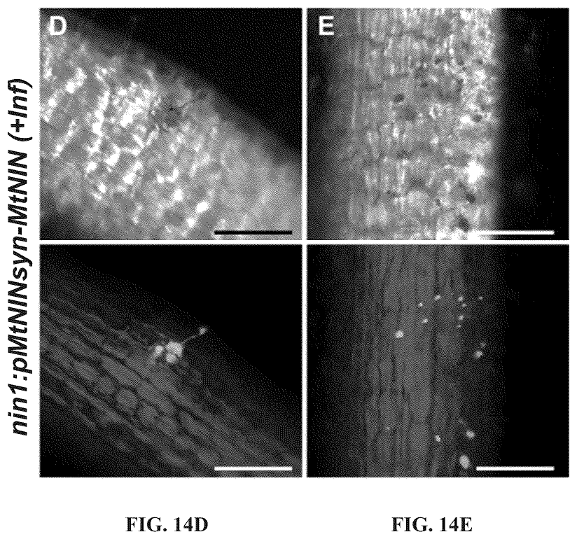

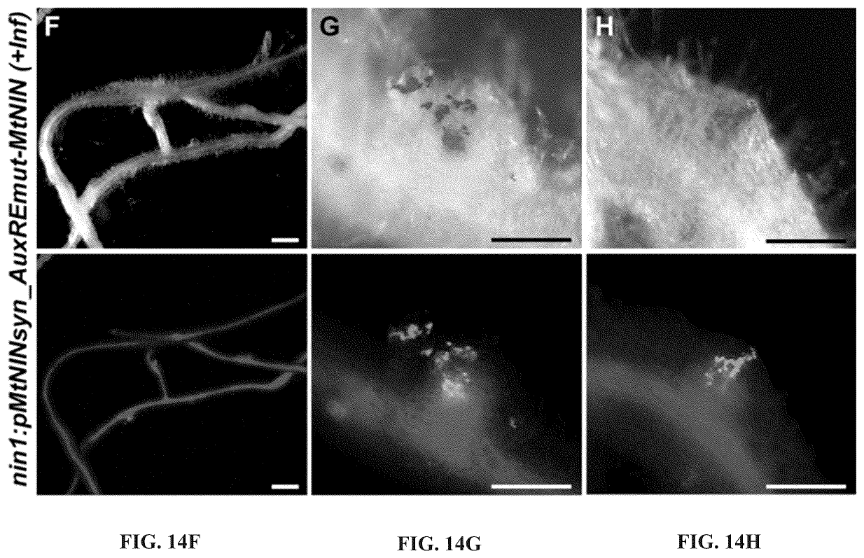

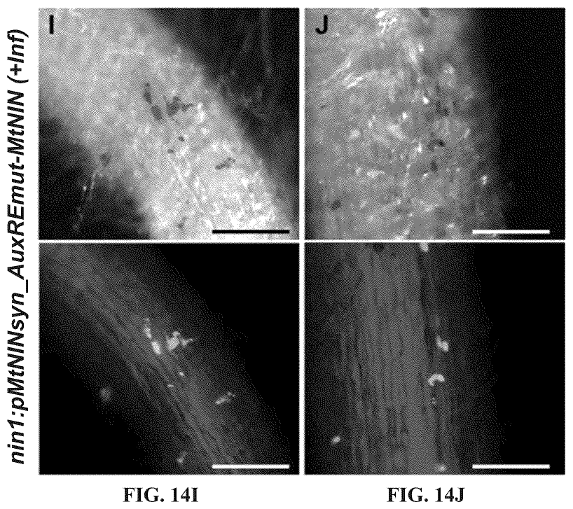

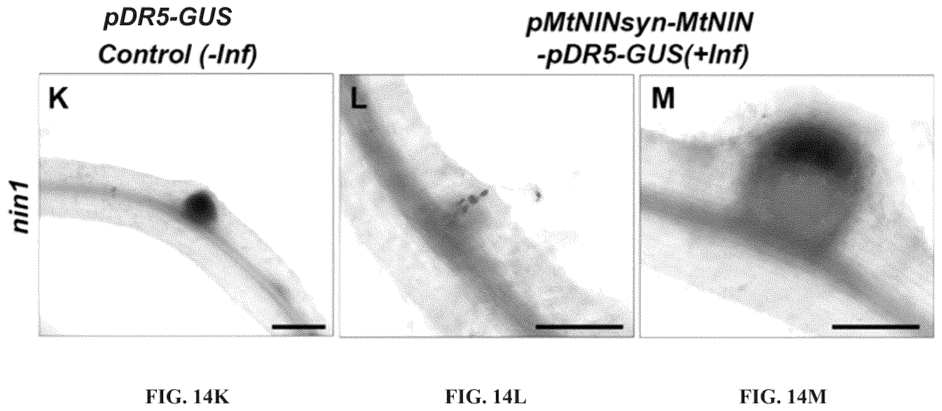

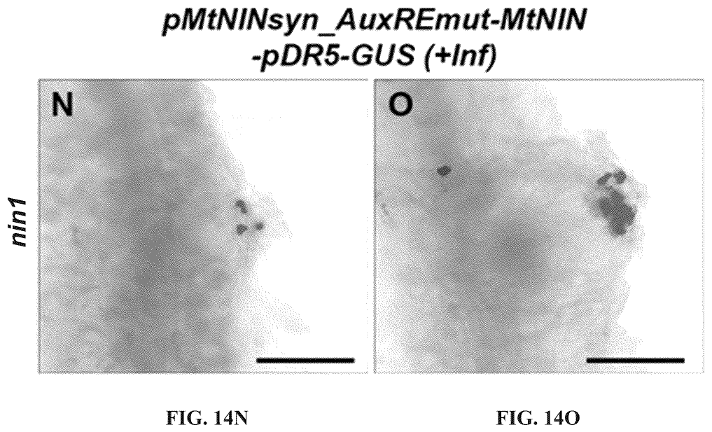

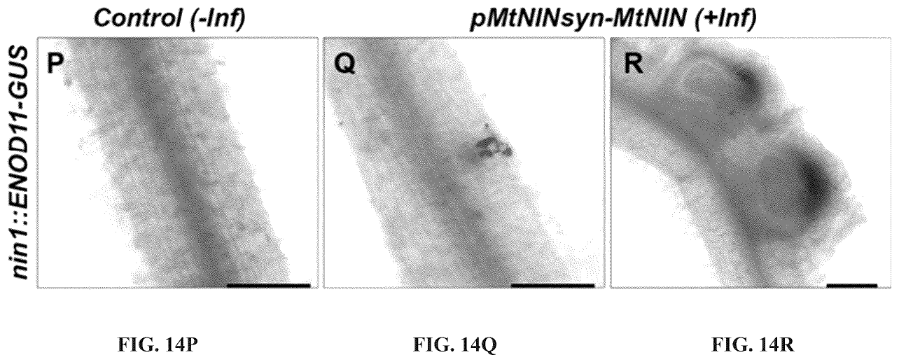

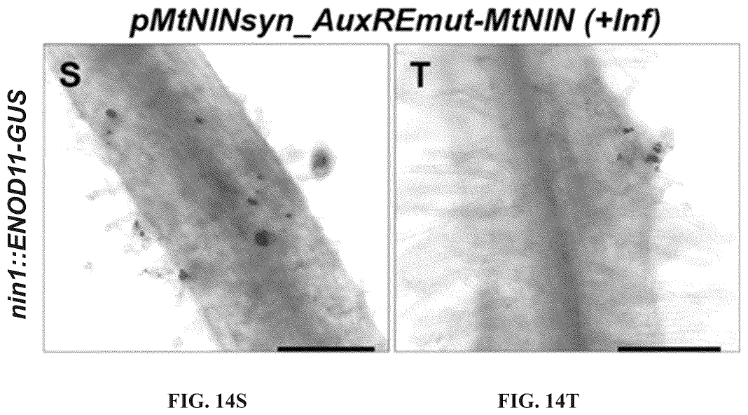

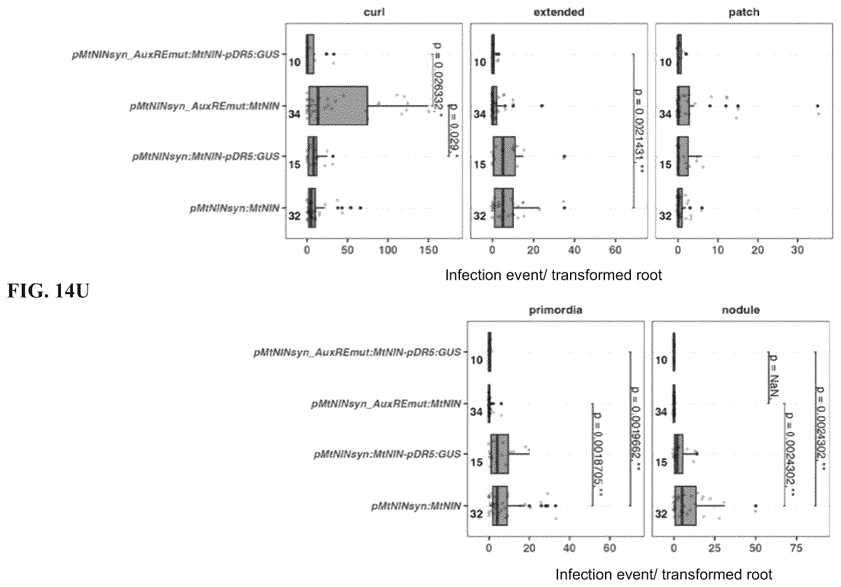

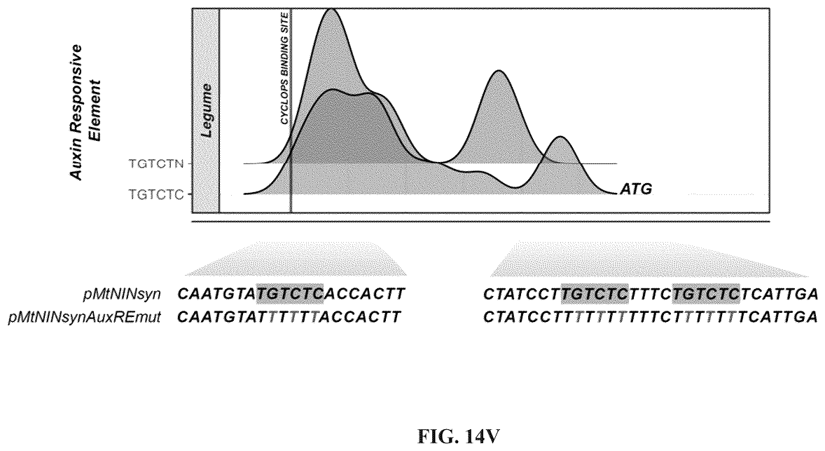

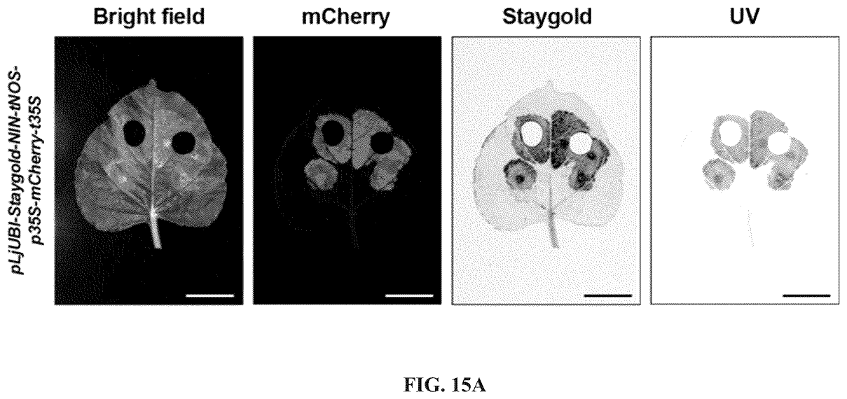

Epidermal auxin induction played a vital role in the infection progression in M. truncatula with a mutation in auxin transporter AUX1 affecting nodule development (Roy et al., 2017). At the same time epidermal induction of NIN is essential for infection progression and induction of cytokinin signalling in the cortex (Vernié et al., 2015). To check whether the auxin responsive elements (AuxRE) in the pMtNIN were essential for coordinating the infection mechanism by controlling the NIN expression, a mutated version of the promoter called pMtNINsynAuxREmut was synthesized (FIG. 14V). In this promoter the three potentially conserved AuxRE sites were mutated while retaining the canonical CycBE and cytokinin responsive elements. pMtNINsynAuxREmut driving MtNIN transformed into M. truncatula nin1 mutant hairy roots showed formation of white nodule with infection patches as compared to proper nodule development observed for non-mutated pMtNINsyn:MtNIN (FIGS. 14A-14B, 14F-14G, and FIG. 14U). At the early stages of infection, pMtNINsynAuxREmut:MtNIN resulted in formation of more infection curls as compared to pMtNINsyn:MtNIN (FIGS. 14E, 14J, and 14U). The lack of significant difference in the formation of extended infection thread between the constructs suggest that early infection is not affected by the mutation of AuxRE (FIGS. 14C-14D, 14H-14I, and 14U). It could be possible that epidermal progression of infection thread requires a threshold of NIN expression which is in turn dependent on auxin accumulation.

Provided the fact that NIN was already known to activate auxin biosynthesis pathways by activating LBD16 expression (Schiessl et al., 2019b; Soyano et al., 2019), auxin accumulation was checked in the transformed hairy roots of M. truncatula nin1 mutants. Constructs carrying pMtNINsyn:MtNIN-pDR5:GUS and pMtNINsynAuxREmut:MtNIN-pDR5:GUS were transformed in M. truncatula nin1 mutants and nodulation was scored four weeks post infection. The pMtNINsyn:MtNIN-pDR5:GUS construct rescued nodulation and showed auxin accumulation both at the early stage of infection and mature nodule development (FIGS. 14L-14M). In comparison, pMtNINsynAuxREmut:MtNIN-pDR5:GUS resulted in nodules with infection thread on its apex and lack of early or late accumulation of auxin substantiated by low pDR5:GUS activity (FIGS. 14N-140). The lack of pDR5 activity in the pMtNINsynAuxREmut:MtNIN roots indicated the reduction in auxin accumulation which in turn affected the MtNIN expression. Interestingly, however, while the infection progression was affected by the AuxRE mutation, the roots still were able to form nodules, suggesting the cortical activity of NIN was unaffected.

NIN negatively affects ENOD11 expression by competing with ERN1 in the epidermis (Xie et al., 2012). To confirm the lack of epidermal activation of MtNIN, the pENOD11:GUS expression was scored in M. truncatula nin1-pENOD11:GUS lines. Hairy-root transformed Mt nin1-pENOD11:GUS plants showed more localized epidermal expression of pENOD11:GUS in pMtNINsyn:MtNIN as compared to diffused epidermal expression in pMtNINsynAuxREmut:MtNIN transformed roots (FIGS. 14Q and 14S). MtENOD11:GUS expression was noted in the distal end of a mature M. truncatula nodule, comprising the infection zone (Journet et al., 2001). Concomitant pENDO11:GUS expression was noted for pMtNINsyn:MtNIN transformed nodules as compared to a more diffused signal in the pMtNINsynAuxREmut:MtNIN primordia, indicating the lack of infection progression (FIGS. 14O and 14T). Thus, it was confirmed that the AuxRE present in the pMtNIN were essential for maintaining an auxin dependent NIN flux controlling the infection mechanism.

Synthetic Auxin Dependent Promoter-Driven Dryas NIN can Induce Nodule-Like Structure in Fragaria vesca.

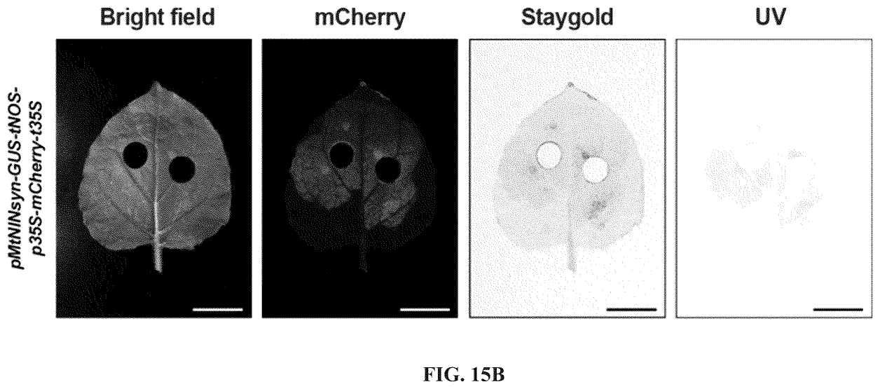

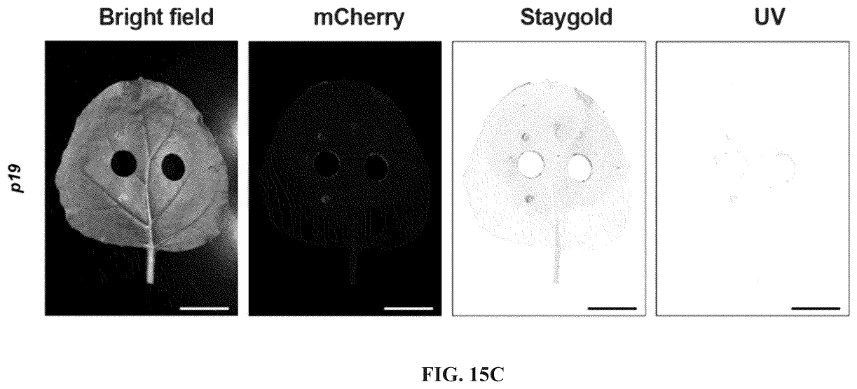

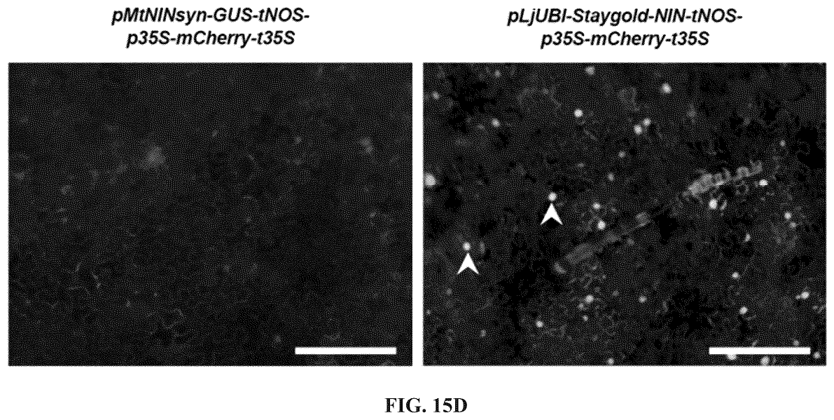

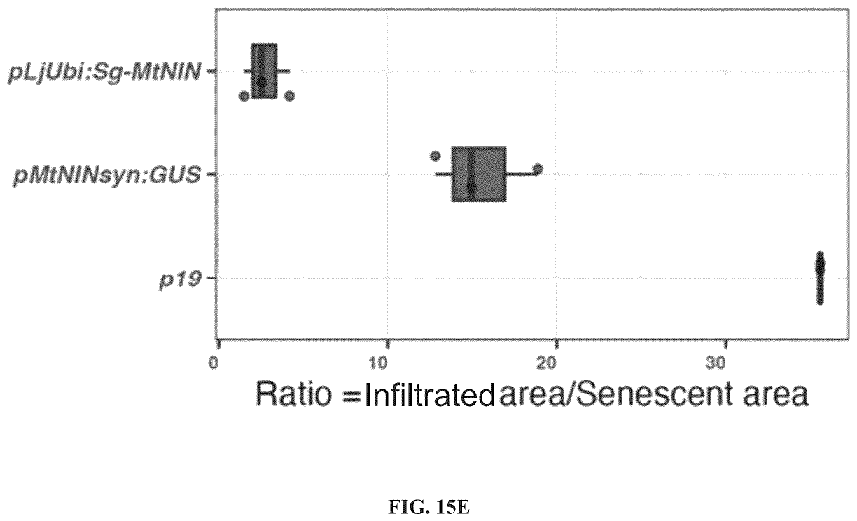

NLPs are responsible for the nitrate responsive lateral root development by directly affecting lateral root organogenesis or by interacting with other factor affecting root formation (Fan et al., 2019; Guan et al., 2017; K. H. Liu et al., 2022). NIN belongs to the NLP clade where it diverged from the NLP1 and, with the loss of the nitrate sensing domain, neofunctionalized to become a regulator of nodulation in the legumes (Suzuki et al., 2013). Previously, overexpression of DgNIN, DgNIN2, and DryasNIN resulted in formation of proliferating structures in the root (FIGS. 7A-7L). Overexpressing DryasNIN in strawberry was therefore tested to see whether it could induce spontaneous cell division in the root. However, attempts to regenerate any shoot from the callus were unsuccessful, and the explants died after some time (Table 5). Similar problems were faced with overexpressing MtNIN in strawberry using an inducible system (Table 5). To investigate the problem further, N-terminal tagged Staygold-MtNIN was overexpressed in Nicotiana leaves (FIGS. 15A-15E). 3 days post infiltration the leaves started to show senescence (FIG. 15A), which was not present in leaves infiltrated with the control construct which did not carry the MtNIN cassette (FIG. 15B), or leaves infiltrated with helper plasmid p19 (FIG. 15C). It was concluded that overexpression of any form of NIN was toxic for the non-nodulating plants.

TABLE 5
Transformation efficiency in strawberry HAWAII4.

| | Number of | Number of calli |
|---|---|---|
| | transformed | with shoots |
| Construct | explants | obtained |
| pLjUBI-GUS-tNOS-p35S- | 50 | 40 |
| mCherry-t35S | | |
| pLjUBI-DdNIN-3xMyc-trbcs- | 50 | 0 |
| p35S-mCherry-t35S | | |
| pDR5-DdNIN_cDNA-tNOS- | 50 | 21 |
| p35S-mCherry-t35S | | |
| pL2B-Kan-pGal-MtNIN-35S- | 38 | 0 |
| pLjUBI-GVG-t35S | | |
| pL2B-Kan-pGal-GUS-t35S- | 38 | 9 |
| pLjUBI-GVG-t35S | | |

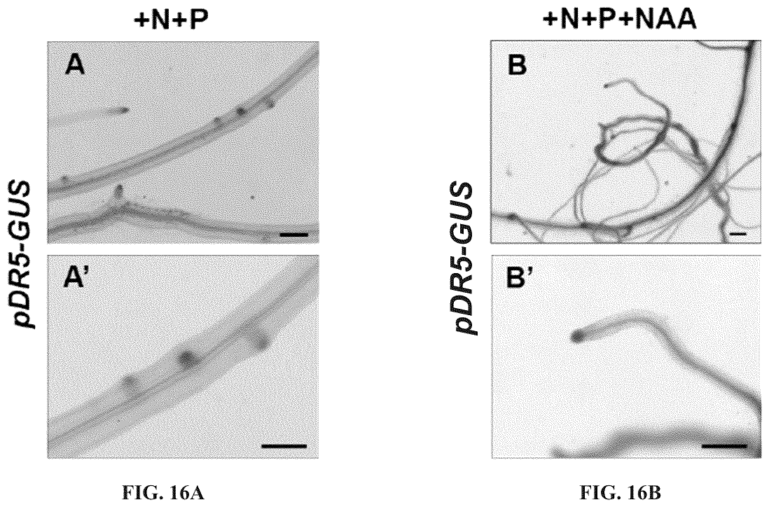

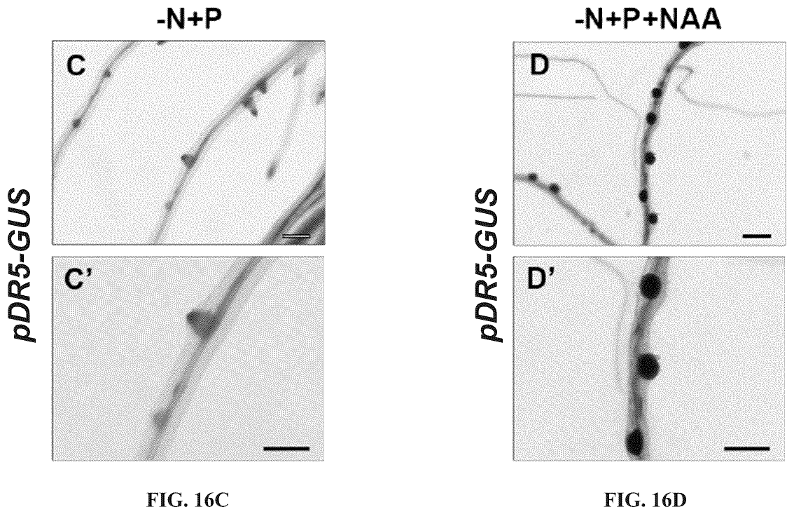

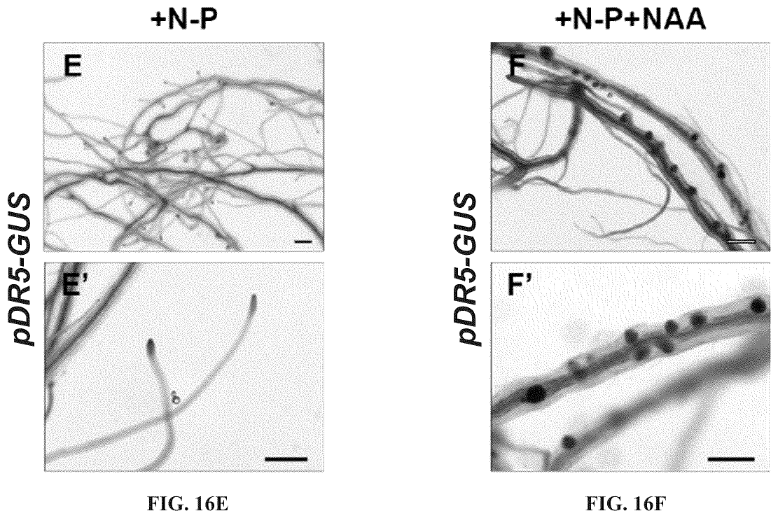

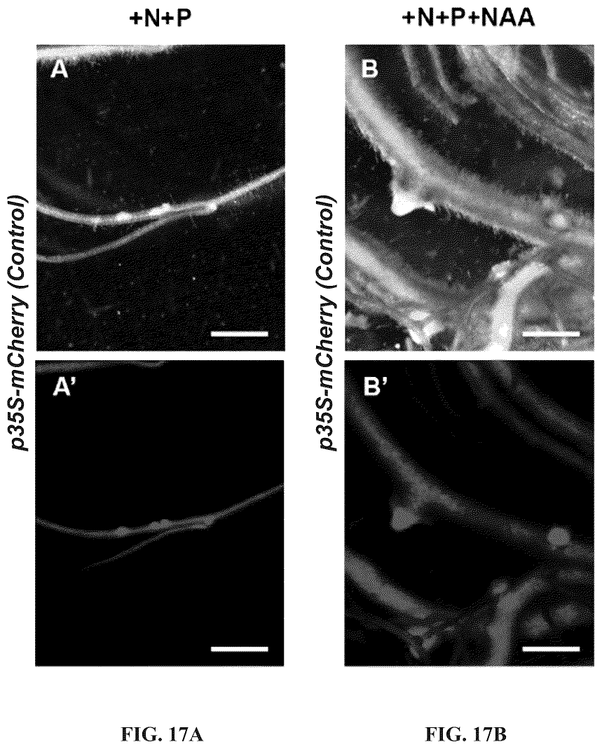

To circumvent the toxicity of NIN overexpression and the possibility of auxin-driven NIN inducing organogenesis in D. glomerata, stable transgenics of diploid strawberry with pDR5:DryasNIN were raised for investigation. To assess the efficacy of the pDR5 promoter in diploid strawberry, stable lines expressing pDR5:GUS were also produced. Dryas NIN was used because D. drummondii is one of the closest nodulators to strawberry, which has experienced loss of nodulation due to loss of NIN (Griesmann et al., 2018). Under the control condition (+N+P) pDR5:GUS showed bright staining in the matured root tip and also at the emerging lateral root (LR) primordia (FIG. 16A). Exogenous treatment with auxin analogue NAA (100⁻⁶M 1-naphthalene acetic acid) resulted in non-specific expression throughout the root system indicating loss of spatial distribution of auxin response (FIG. 16B). Previous evidence mostly in Arabidopsis shows that nitrate level plays an important role in LR development (H. Zhang & Forde, 1998). This LR development is in turn controlled by a passive feedforward mechanism involving auxin signaling pathway (Vidal et al., 2010). Nitrogen (N) starvation of strawberry plants resulted in activation of the auxin signaling pathway which is evident from the intense GUS staining of the roots (FIG. 16C). Under the N limiting (−N+P) condition the vascular bundle and the entire lateral root primordia shows GUS staining, highlighting the heightened auxin signaling (FIG. 16C). Exogenous auxin treatment under the N limiting condition resulted in a complete spatial shift in the auxin response. Unlike any of the individual conditions previously observed for +N+P+NAA (FIG. 16B) or −N+P (FIG. 16C), −N+P+NAA resulted in localized GUS staining in the bump like structures (FIG. 16D). A very similar spatial shift in the GUS staining profile was also observed under phosphate (P) limiting condition post auxin treatment (FIGS. 16E-16F). P starvation was directly linked with lateral root formation with the activation of auxin signaling pathway (Crombez et al., 2019). Thus, these data established that nutrient limiting condition coupled with auxin treatment can differentially activate the auxin signaling pathway in strawberry.

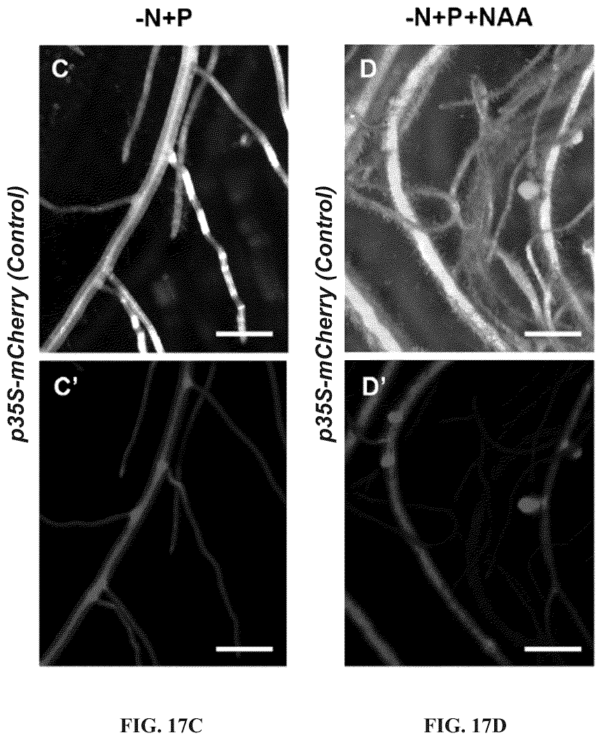

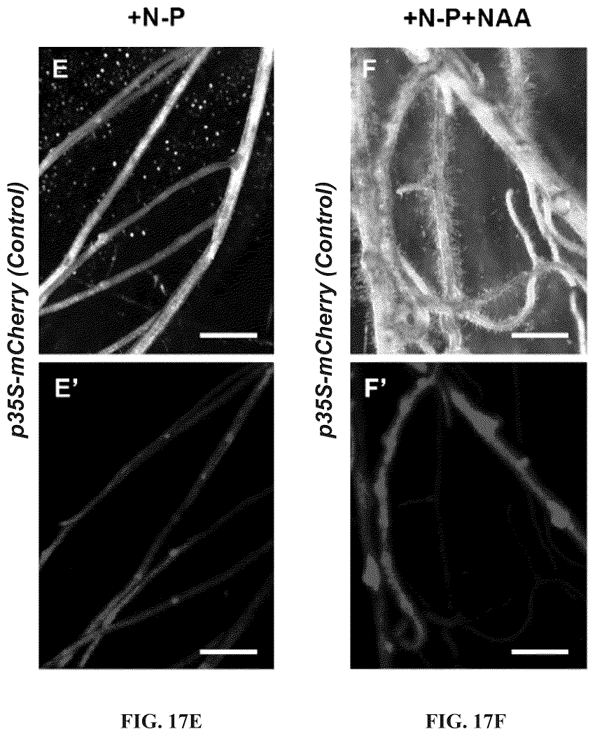

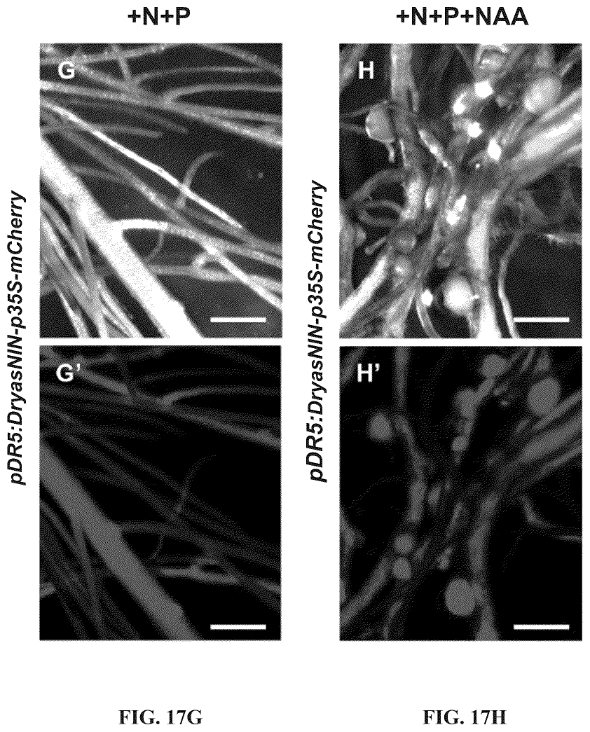

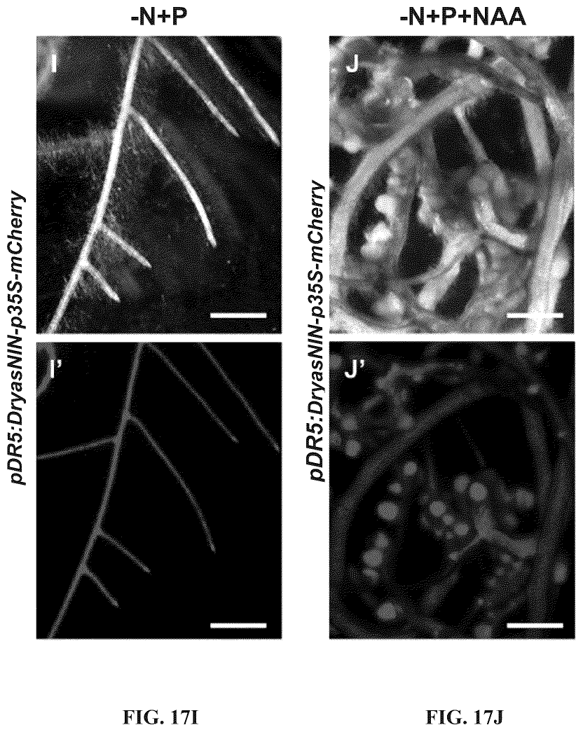

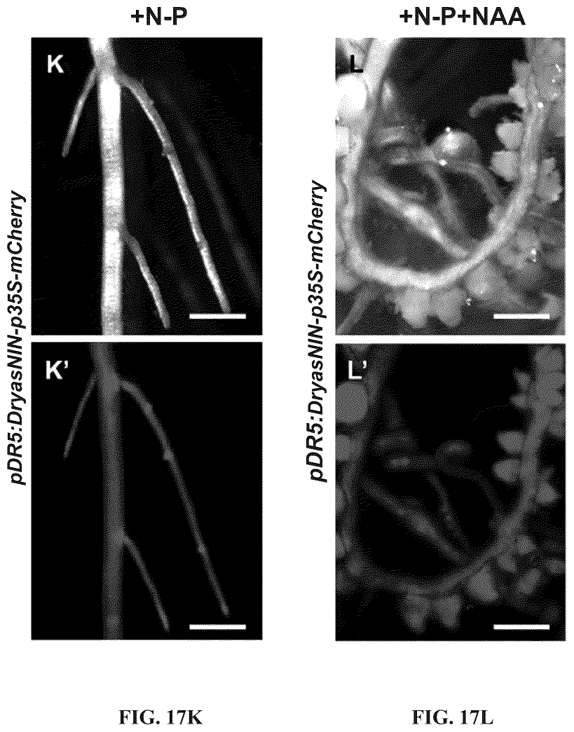

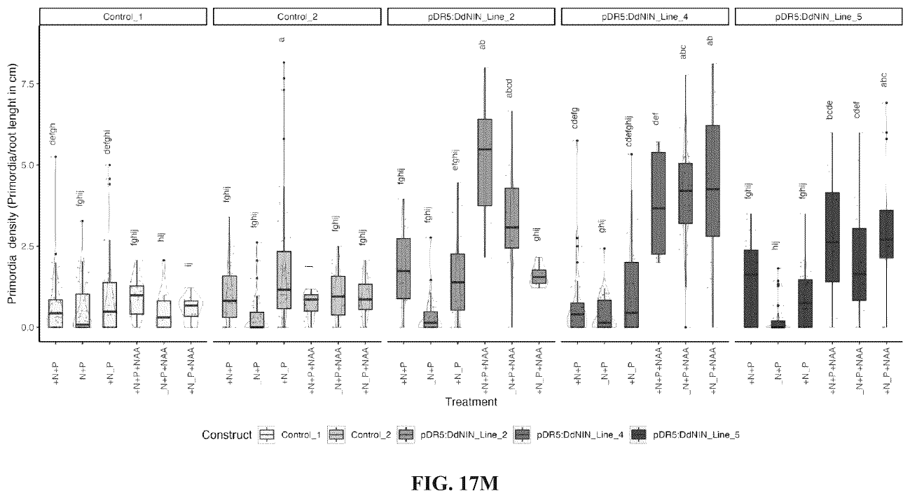

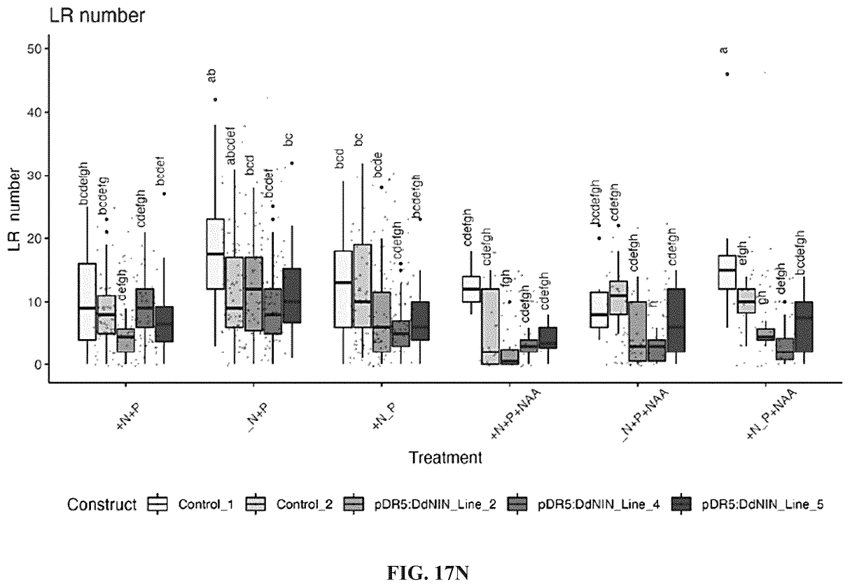

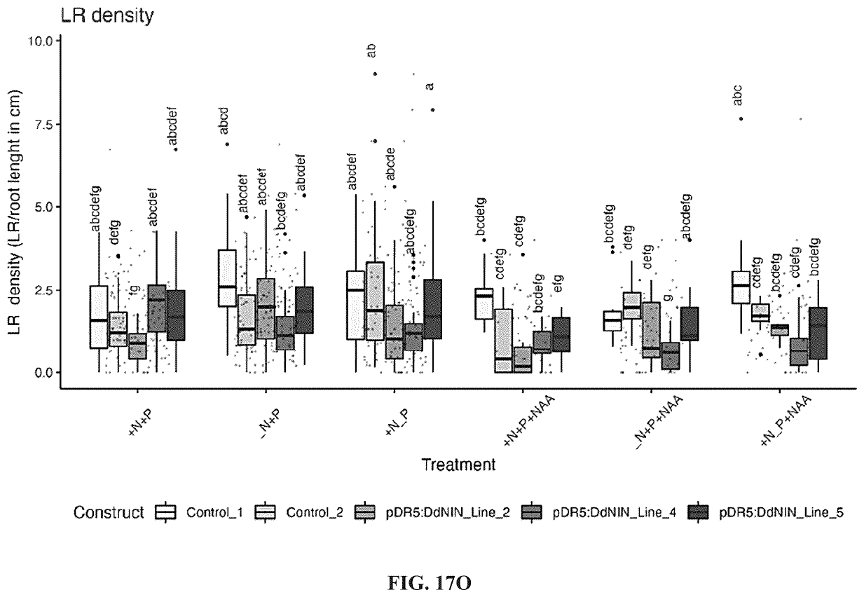

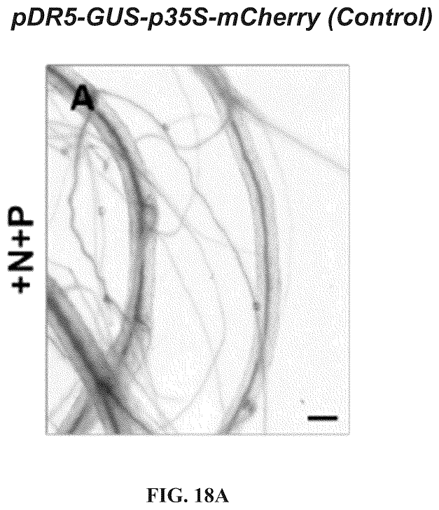

The strawberry transgenic lines expressing pDR5:DryasNIN were treated with auxin under both N and P limiting condition as well as repleted condition for 4 weeks (FIGS. 17A-17M). Under the +N+P+NAA condition, the pDR5:DryasNIN lines (FIGS. 17G-17H) showed formation of numerous bump-like structures on the roots which were significantly higher (FIG. 17M) than the control transformed lines (FIGS. 17A-17B). The structures were more round as compared to the similar bump-like structures formed on the control transformed lines indicating a change in the development of these structures (FIGS. 17B and 17H). The N limiting and P limiting condition induced more lateral root formation (FIGS. 17N-170). Under the −N+P+NAA and +N−P+NAA condition similar induction of the bump-like structures was observed, and in pDR5:DryasNIN line 4 both were significantly higher as compared to +N+P+NAA condition (FIG. 17M). Under the effect of NAA treatment under all the nutrient condition pDR5:DryasNIN lines produced significantly higher numbers of bump-like structures which often resemble nodule-like structures (FIGS. 17C-17F and 17I-17M). Auxin treatment did have a negative effect on the total number of elongated lateral roots as compared to the untreated condition (FIG. 17N). It is very likely that the reduction in the elongated lateral root is compensating for the number of bump-like structures formed on the root upon auxin treatment. It is noteworthy that even under control condition pDR5:DryasNIN lines had fewer LR as compared to the control lines (FIG. 17N). But there was not much significant difference in the density of the LRs (FIG. 17O), indicating that the primary root lengths of the pDR5:DryasNIN lines were shorter than in the control lines. Thus, this established that in a non-nodulating plant like strawberry, expression of NIN in the presence of auxin can induce organogenesis of nodule like structures (NLS).

Ultrastructure Analysis of the NLS in Strawberry Indicates their Change in the Fate Map.

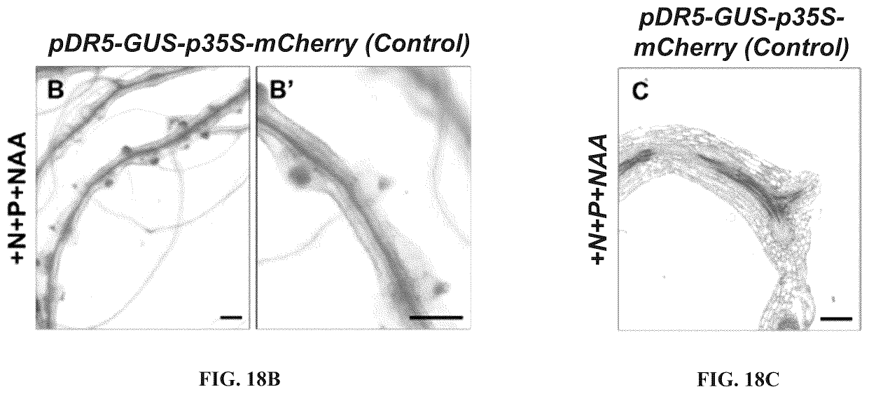

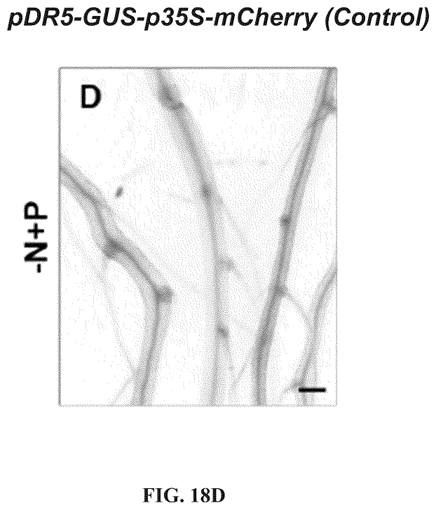

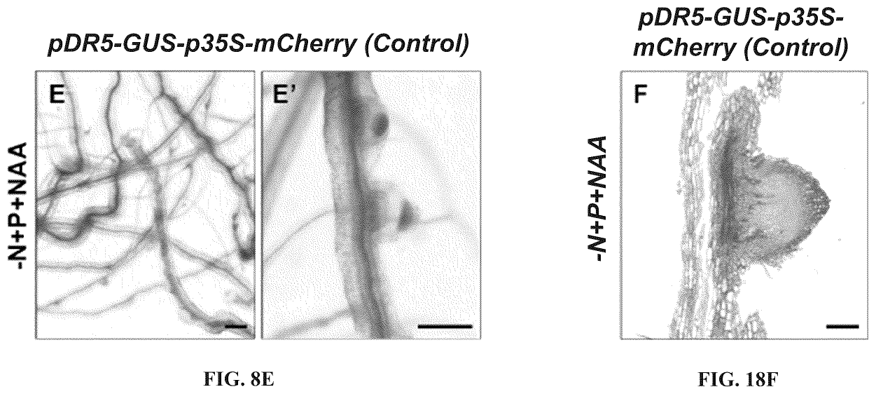

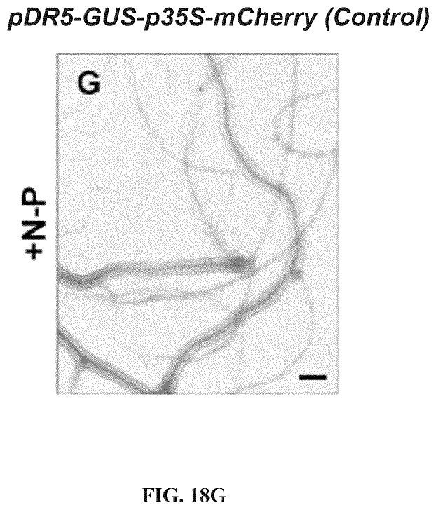

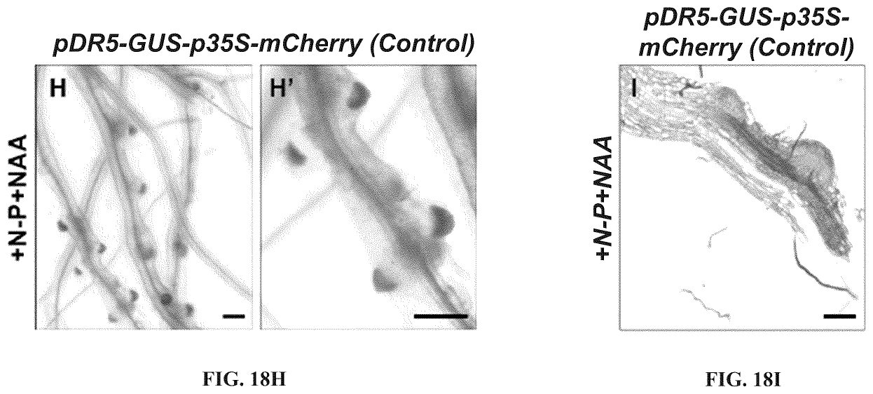

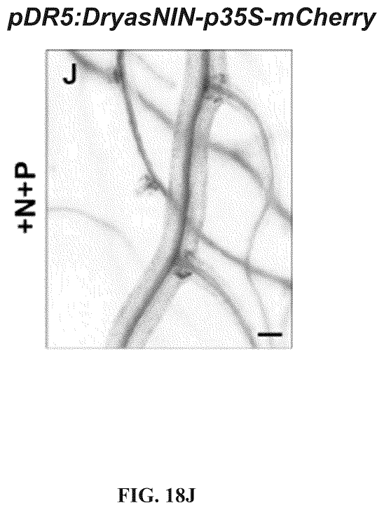

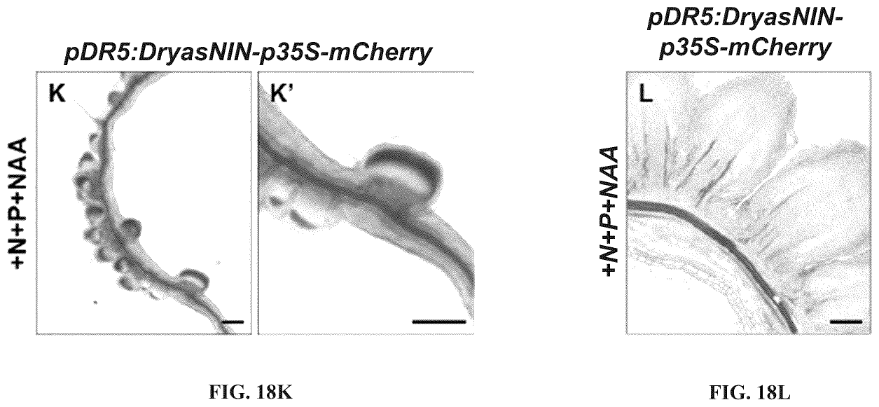

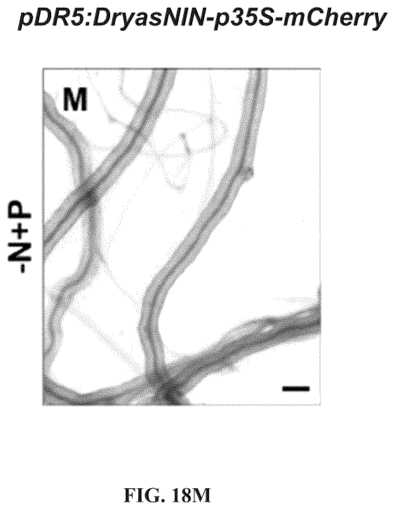

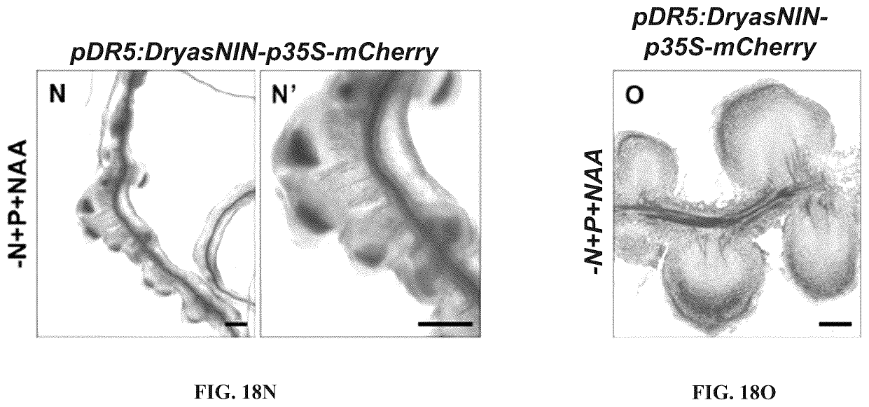

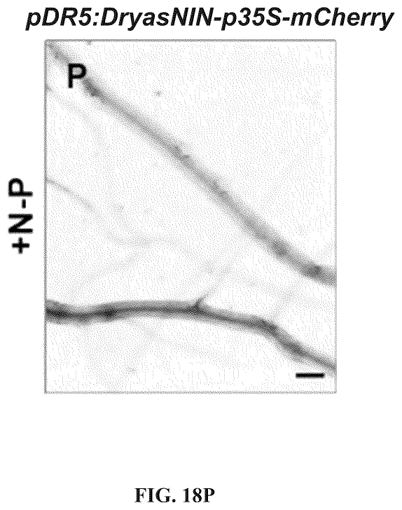

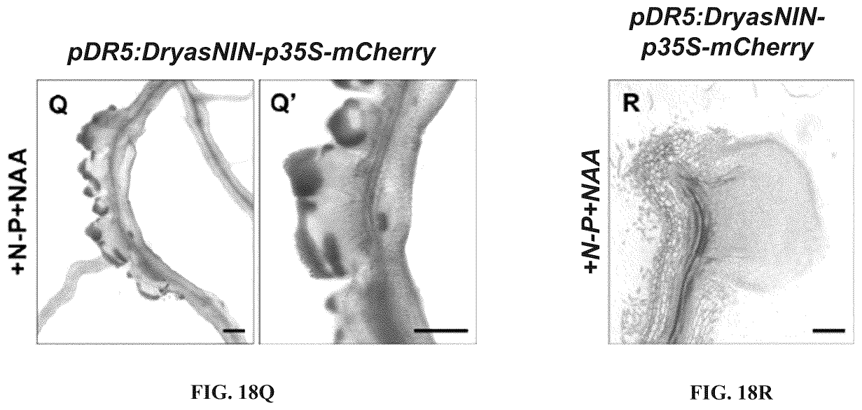

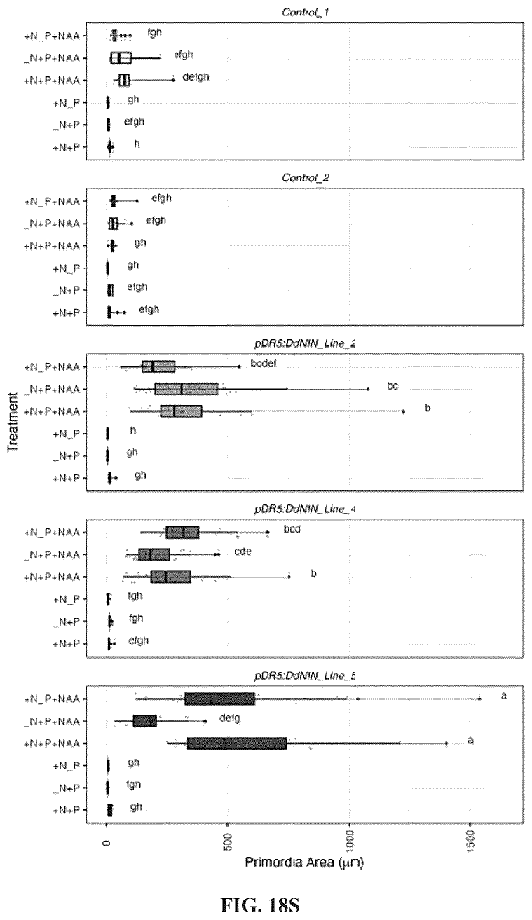

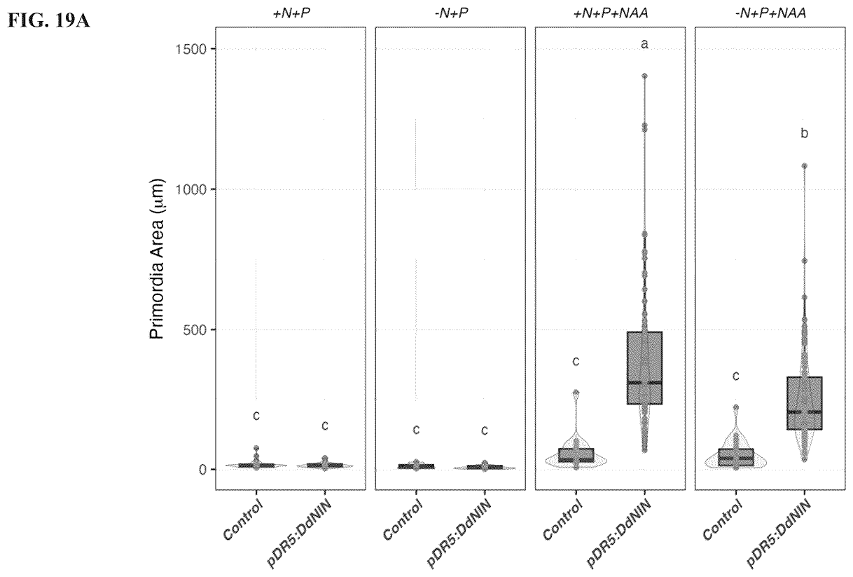

Auxin and cytokinin both play pivotal roles during nodulation where auxin is primarily responsible for inducing neocortical cell division (Kohlen et al., 2018b; Ng et al., 2015). In a recent report it was also found that the role of cytokinin in inducing spontaneous nodule formation is restricted to legumes and it has no effect on the actinorhizal plants (Gauthier-Coles et al., 2019). On the other hand, it was determined that auxin responsive NIN could induce spontaneous nodule like structures in D. glomerata (FIGS. 10A-10N). Closer examination of the NLS in the pDR5:DryasNIN lines indicated that the total size of the structures was significantly higher as compared to the control lines (FIGS. 18A-18S). While even the control lines produced some NLS resembling the ones observed in the pDR5:DryasNIN lines, they are significantly fewer in number and more importantly they never formed merged structures (FIGS. 18B, 18E, and 18H, compared to 18K, 18N, and 18Q). The formation of the merged structures indicated a spatial loss of the lateral root patterning mechanism. Ultrastructure analysis of the structures showed the presence of elaborate vascular bundles which were often diffused and at other times peripheral (FIGS. 18L, 18O, and 18R). The presence of the peripheral vasculature was quite significant as the NLS in the control lines are mostly central and rarely form some diffused vasculature (FIGS. 18C, 18F, and 18I). Formation of diffused vasculature in the spontaneous nodule like structures was previously reported in M. truncatula (Rightmyer & Long, 2011). It is very likely that the peripheral vascular bundle observed in the pDR5:DryasNIN lines indicated a change in the fate map of these structures where they cease to have the lateral root identity due to the presence of NIN.

Identifying Hallmarks for Proto-Nodule Through Transcriptomic Analysis of NLS in pDR5:DryasNIN Lines of Strawberry.

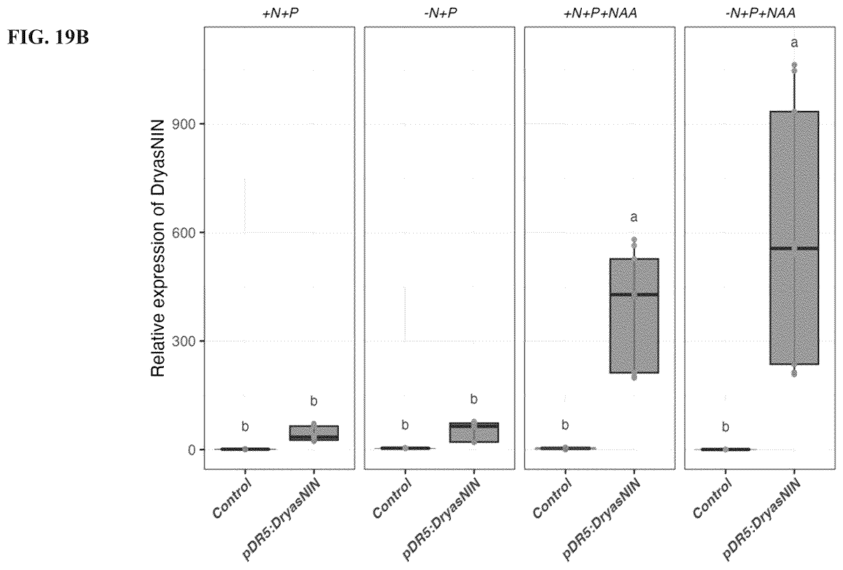

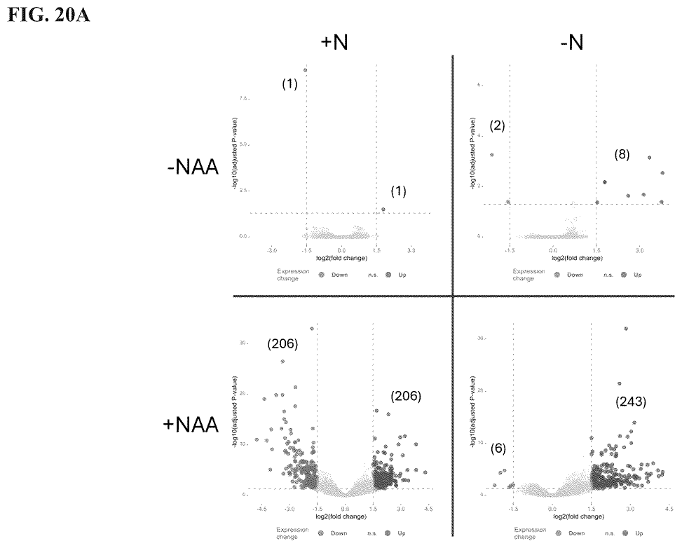

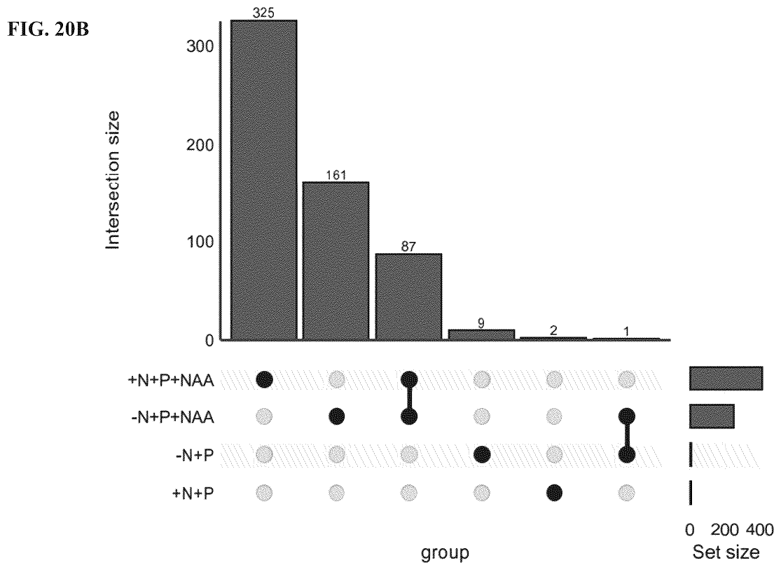

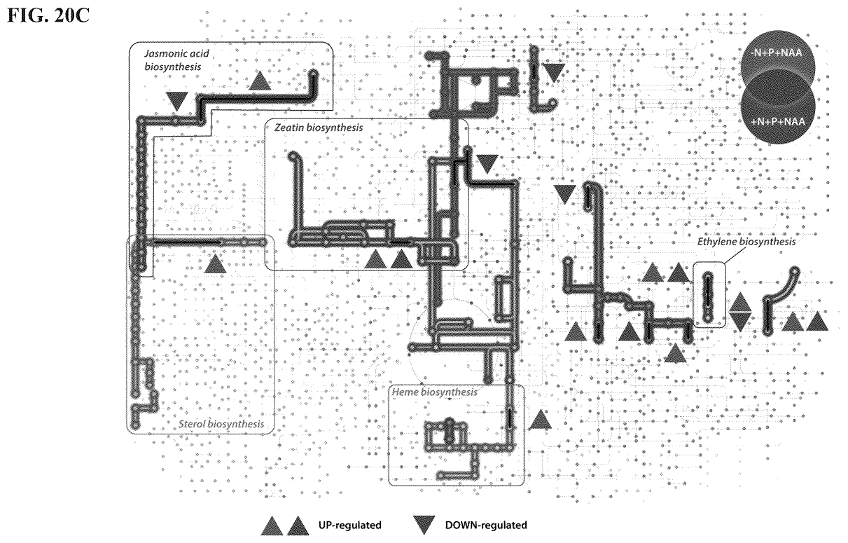

The NLS formed on the strawberry roots of pDR5:DryasNIN lines following auxin treatment were novel structures. To investigate whether these structures had any molecular identity of a nitrogen fixing nodule, transcriptomic analysis was done with total RNA extracted from the root under different conditions. A collinearity was seen between the structures formed in the strawberry root and the expression of DryasNIN (FIGS. 19A-19B). Differentially Expressed Genes (DEGs) were identified by contrasting the RNAseq data from the pDR5:DryasNIN lines to the control lines under the 4 different conditions: +N+P, −N+P, +N+P+NAA, and −N+P+NAA. A volcano plot showed that under +N+P and −N+P conditions there were not many DEGs (FIG. 20A-20B). On the other hand, the +N+P+NAA condition showed 206 up-regulated and 206 down-regulated genes (FIG. 20A). The −N+P+NAA condition showed 243 up-regulated genes, as compared to only 6 downregulated genes. 87 DEGs were found to be represented in both +N+P+NAA and −N+P+NAA conditions, indicating their independence of the N status (FIG. 20B).

Gene set enrichment analysis indicated that the DEGs under NAA conditions represented certain metabolic pathways (FIG. 20C), of which the most significant are the Jasmonic acid biosynthesis, Zeatin biosynthesis, and ethylene biosynthesis pathways, which are affected under both +N+P+NAA and −N+P+NAA conditions (FIG. 20C). Comparatively, under the −N+P+NAA condition DEGs affecting the sterol biosynthesis and heme biosynthesis were notable. In both cases, genes upstream in the pathway were significantly upregulated (FIG. 20C).

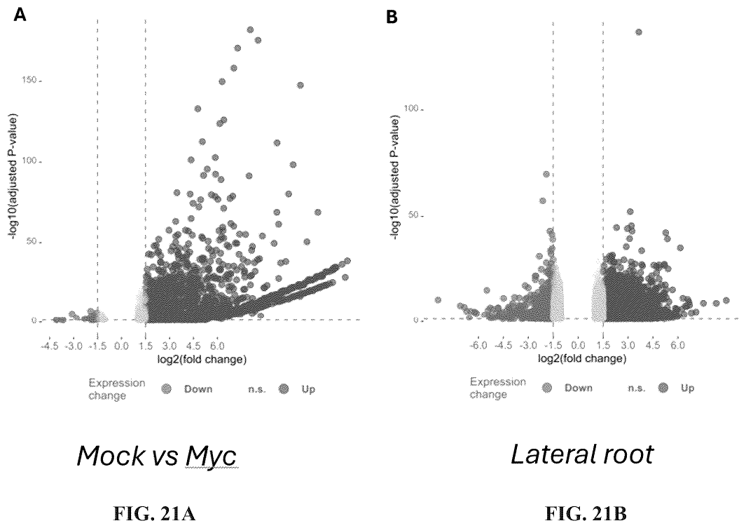

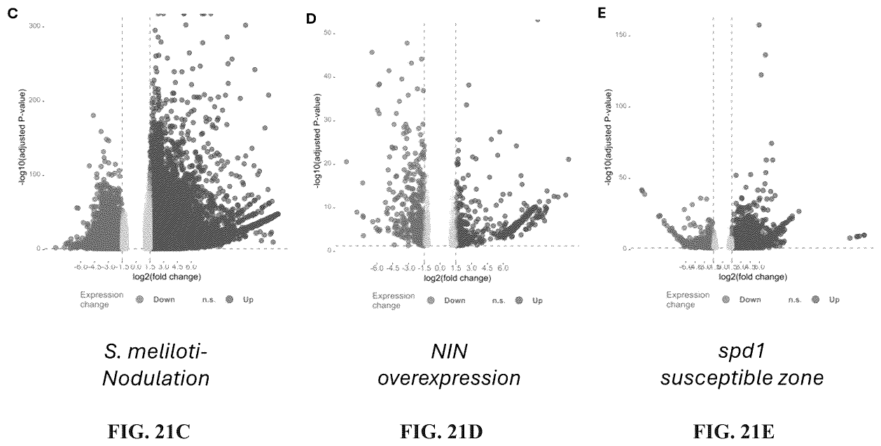

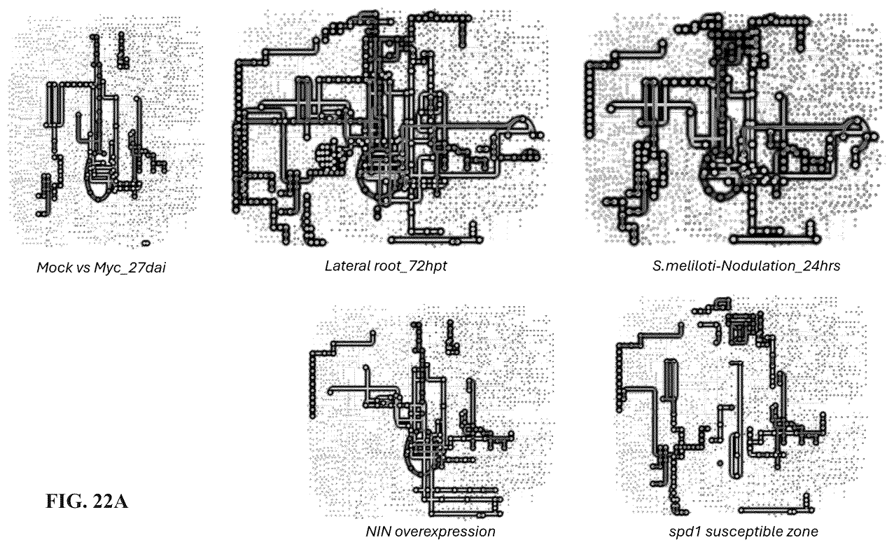

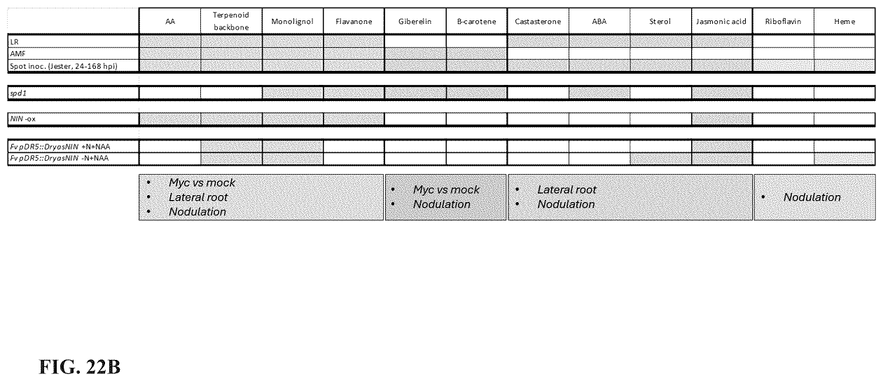

To understand the significance of the DEGs in the roots with NLS in strawberry, the available transcriptomes of M. truncatula were analyzed, including: Mock vs Myc—DEGs activated during mycorrhization; Lateral root—DEGs involved in lateral root development; Nodulation—spot inoculation with S. meliloti; Spontaneous nodulation mutant—NIN overexpression; and spd1 (spontaneous nodule development) susceptible zone (FIGS. 21A-21C). These datasets were selected to identify the metabolic pathways that are affected during mycorrhization (Mock vs Myc), organogenesis (Mock vs Myc and nodulation), root organ development (lateral root development and nodulation), and nodule organogenesis devoid of infection (spontaneous nodulation in NIN overexpression and spd1). Gene set enrichment analysis showed that there were multiple pathways that were commonly represented between the above-mentioned conditions and strawberry NLS data (+N+P+NAA and −N+P+NAA) (FIGS. 22A-22B). The most significant of these pathways include Terpenoid biosynthesis, Monolignol biosynthesis, Sterol biosynthesis, Jasmonic acid biosynthesis, and Heme biosynthesis pathways (FIG. 22B). Of these, the Sterol, Jasmonic acid, and Heme biosynthesis pathways are common for lateral organ development and spontaneous nodulation. Thus, from the comparative transcriptomic analysis a certain signature was determined that could justify the unique proto-nodule identity of the NLS structures in strawberry.

### Example 2: Dryas NIN was Able to Induce Organogenesis in Barley with Auxin Treatment

The following example describes assays to test the effects of pDR5:DdNIN in barley.

### Materials and Methods

The experiments described in this Example are performed as described in Example 1 unless otherwise noted.

### Barley Stable Lines

### A. Plant Growing Conditions

Barley (Hordeum vulgare L. cv. Golden Promise) were raised using A. tumefaciens-mediated transformation (Bartlett et al., 2008). Barley seeds were treated with 70% ethanol for 2 minutes, followed with three times wash in sterile water. The seeds were then surface sterilized by 5% sodium hypochlorite solution for 4 minutes and rinsed with sterile water 4-5 times. The sterilized seeds were plated on 1% water agar plates and imbibed at 4° C. for 3 days, then germinated in the dark at 22° C. for 2-3 days (Li et al., 2022). STARTS transgenics were raised using A. tumefaciens-mediated transformation (Imani et al., 2011).

### B. Hormone Treatment of Barley

Tissue culture STARTS plants were grown in rooting media for approximately 4 weeks after transfer, and barley seeds were grown on water-agar plates for seven days after germination, after which seedlings were transferred to 1% Hoagland's media plates. Seedlings were treated under control conditions, nitrogen deficient conditions, and each of the above in combination with the addition of exogenous auxin analogue NAA (100⁻⁶M 1-naphthalene acetic acid), 0.8% agarose (Sigma). The root portion of the plates was covered with aluminum foil. The plants were maintained under a 16 h-day and 8 h-night cycle at 24-22° C. Tissue was harvested four weeks after treatment for phenotyping and RNA extraction.

Phenotypic Analysis—H. vulgare Whole Transgenics and STARTS

Whole plants were imaged using a stereo microscope DX105 F (Leica) using LAS 3.4 software.

### Results and Discussion

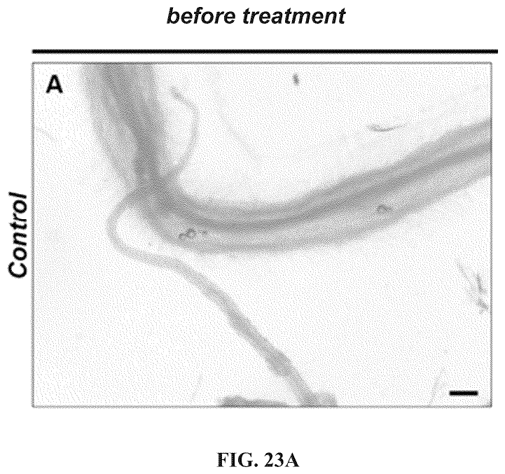

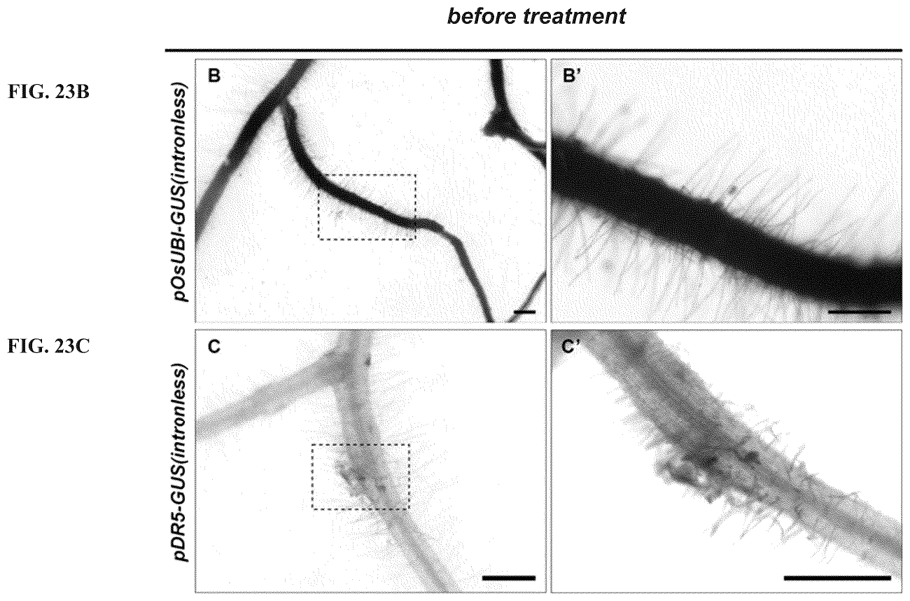

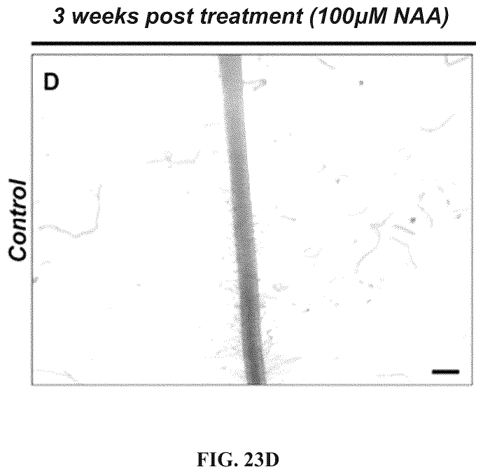

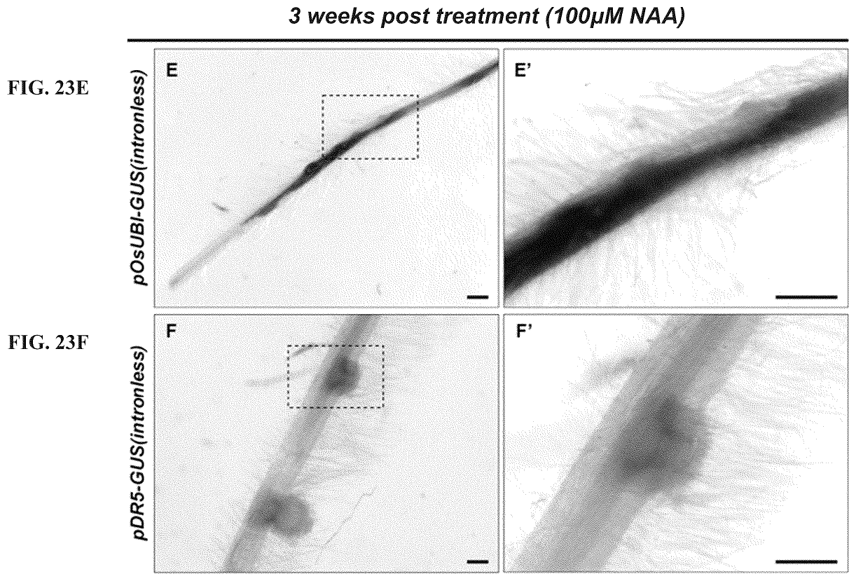

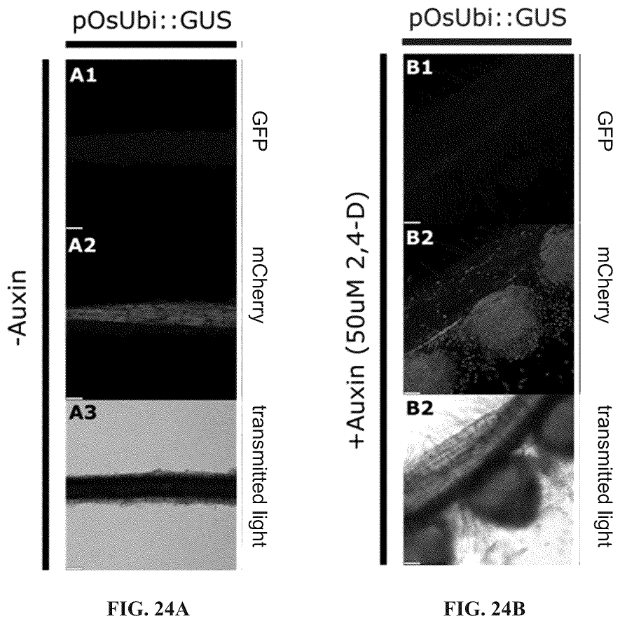

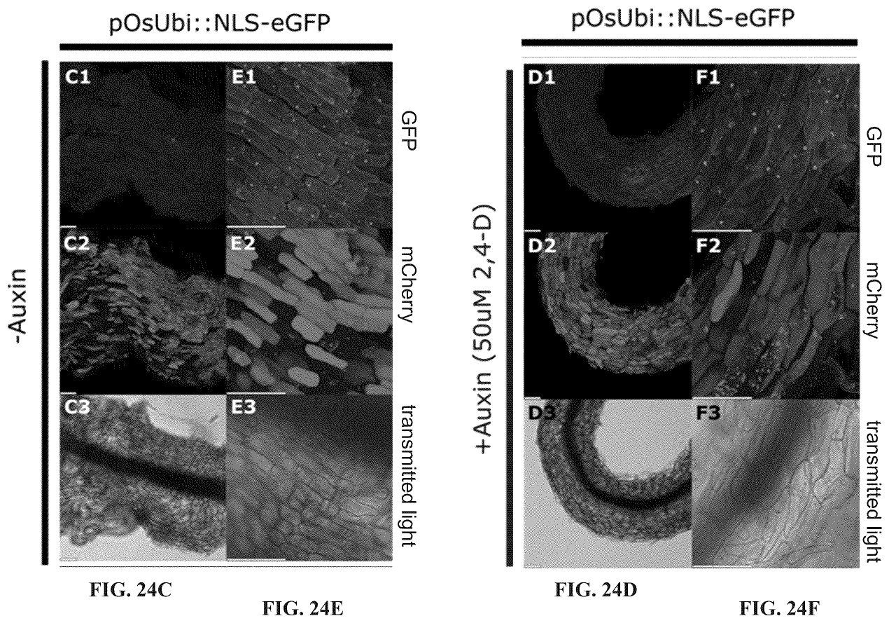

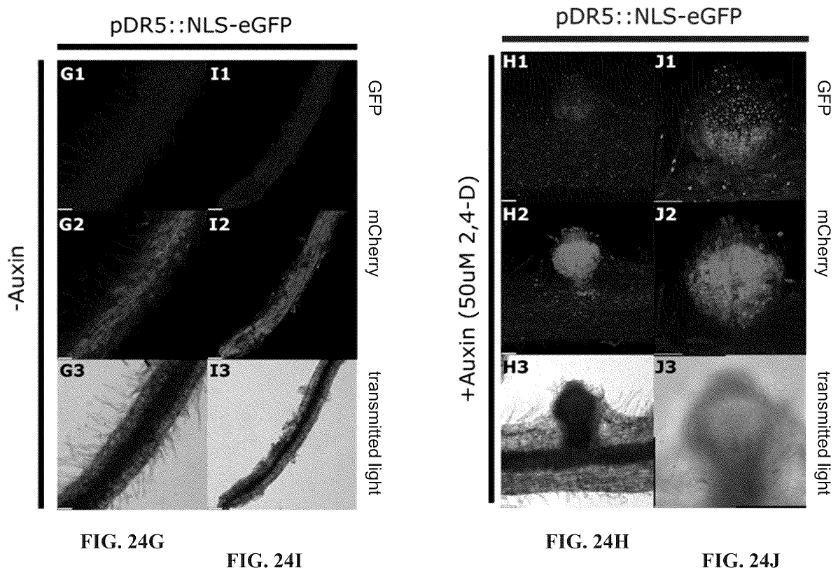

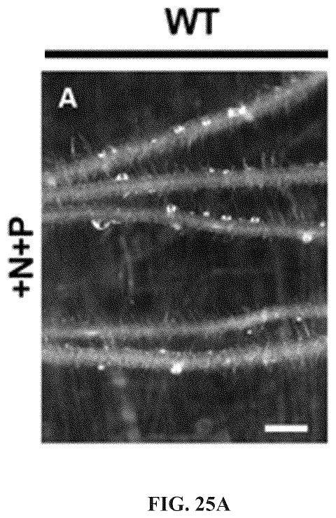

Auxin treatment was reported to induce nodule like-structures (NLS) in rice (Hiltenbrand et al., 2016). Since it was established in the strawberry system that expressing pDR5:DryasNIN could form NLS with some proto-nodule identity under the influence of auxin, lines in barley (Hordeum vulgare var Golden promise) were raised with the same cassette. To establish the efficacy of the pDR5 promoter in barely system, some transgenics were also raised with pDR5:GUS and pDR5:NLS-eGFP (FIGS. 23A-23F, 24A-24J). In comparison to overexpressing pOsUbi:GUS, pDR5:GUS expression under control condition was very weak (FIGS. 23B-23C). However, with 100 μM NAA (auxin) treatment GUS staining was visible in the bump-like structures (FIGS. 23E-23F), indicating its responsiveness to auxin treatment. The GUS staining pattern was corroborated by the pDR5:NLS-eGFP data where eGFP localization could be seen in the nucleus after 50 μM 2,4D (auxin) treatment (FIGS. 24A-24J, top sections).

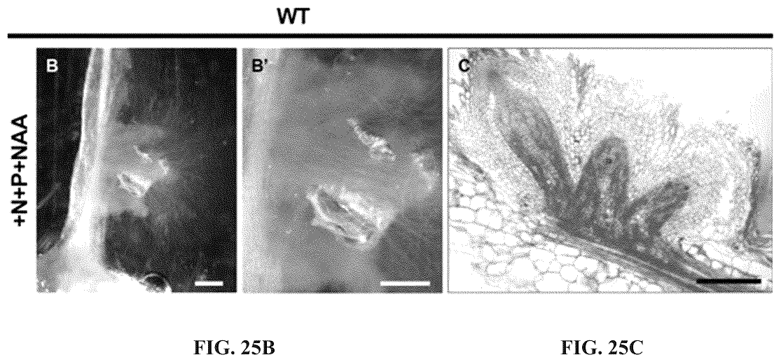

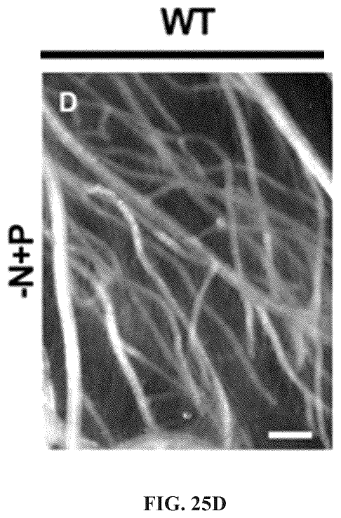

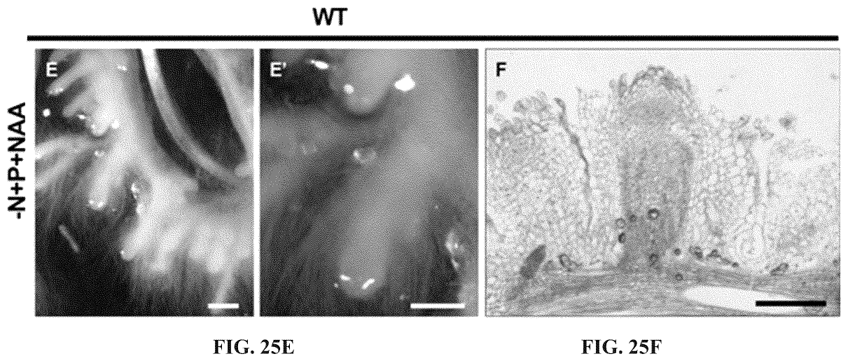

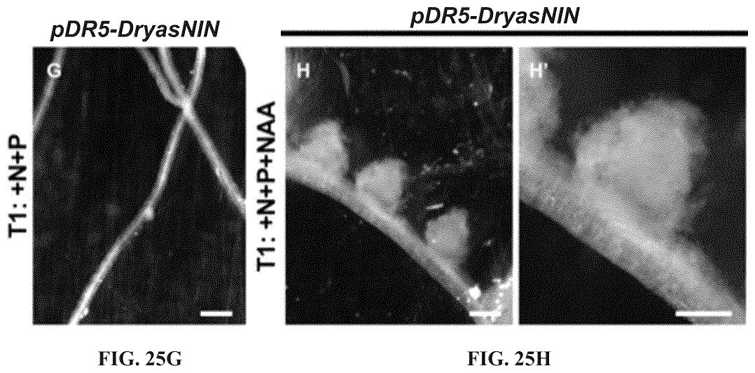

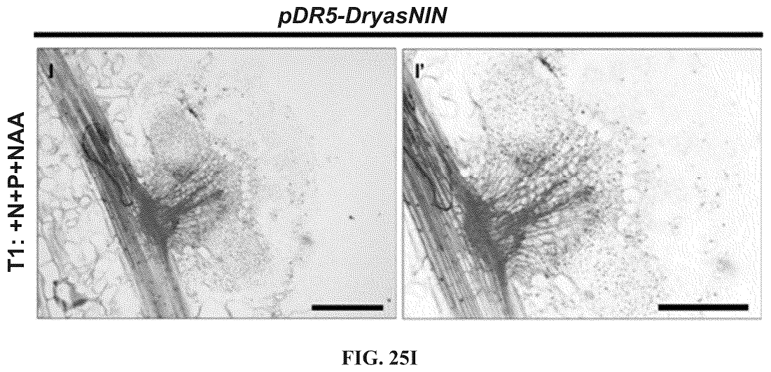

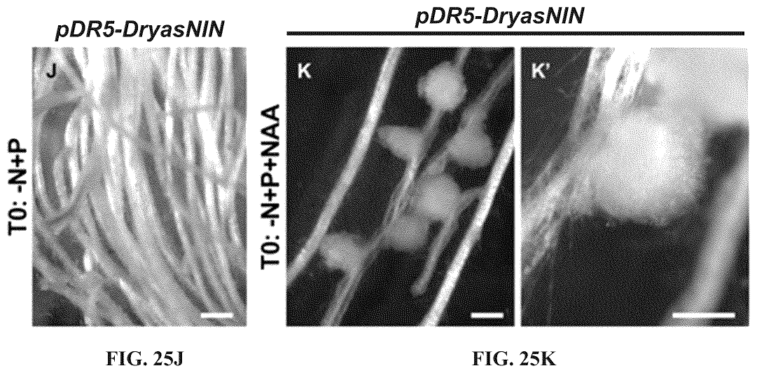

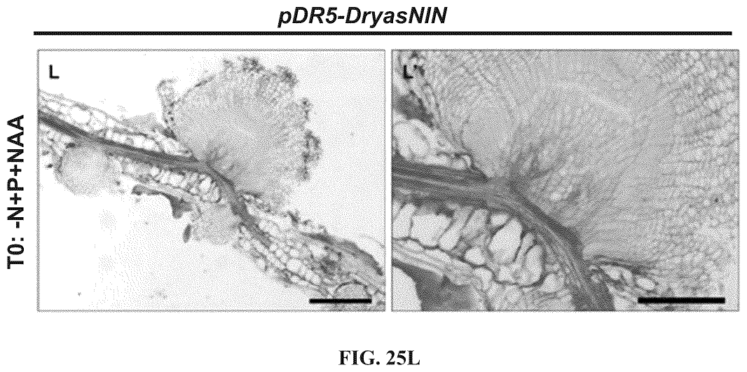

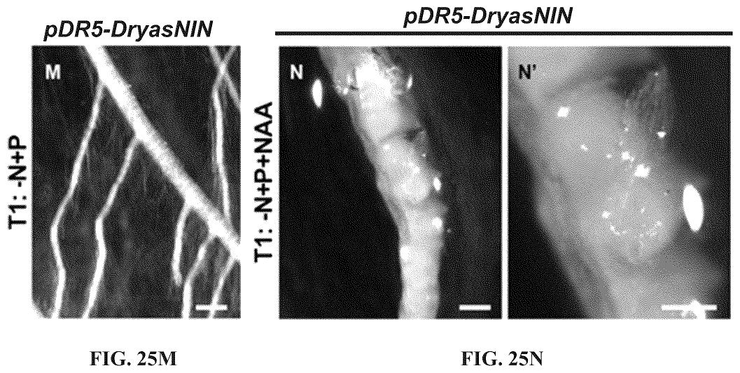

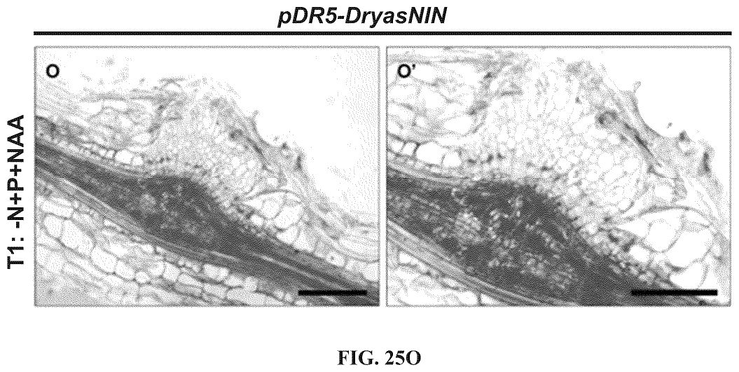

Having established the auxin responsiveness of the pDR5 promoter, the pDR5:DryasNIN barley lines were treated with auxin (100 μM NAA) under both N repleted (+N+P+NAA) and depleted (−N+P+NAA) conditions (FIGS. 25A-25O). It is important to note that the TO and T1 lines with pDR5:DryasNIN showed the formation of NLS after 4 weeks of treatment (FIGS. 25G-25O). Ultrastructure analysis of the NLS showed the presence of multiple vascular bundles that were often peripheral as compared to a more central vasculature in the bump-like structure of control plants (FIGS. 25A-25F compared to FIGS. 25G-25O). The formation of the NLS indicated the cross species potential of the pDR5:DryasNIN in inducing NLS.

### Example 3: Spatiotemporal Expression Pattern of DgNIN2 During Intercellular Actinorhizal Nodule Development

The following example describes the spatiotemporal expression pattern of Datisca glomerata NIN2 during intercellular actinorhizal nodule development.

### Materials and Methods

The experiments described in this Example are performed as described in Example 1 unless otherwise noted.

### Results and Discussion

To check whether there is any conservation in the expression pattern of the NIN2 promoter between legume and actinorhizal plants, the pDgNIN2_5kb:GUS is also transformed into the hairy root of M. truncatula A17 plants, and expression of GUS is assayed.

### Example 4: Complementation Assay of Actinorhizal DgNIN2 for Medicago Mutant Nin1

The following example describes assays to assess the ability of DgNIN2 to complement the Medicago nin1 mutant.

### Materials and Methods

The experiments described in this Example are performed as described in Example 1 unless otherwise noted.

### Results and Discussion

To check whether there is cross-species conservation of the NIN protein function, rescue of the nin1 mutant is tested by hairy root transformation using the synthetic M. truncatula NIN promoter driving the DgNIN2. As a readout of the rescue efficiency, infection thread phenotype and the nodule organogenesis are scored.

### REFERENCES

Bartlett, J. G., Alves, S. C., Smedley, M., Snape, J. W., & Harwood, W. A. (2008). High-throughput Agrobacterium-mediated barley transformation. Plant Methods, 4(1).
Bertioli, D. J., Cannon, S. B., Froenicke, L., Huang, G., Farmer, A. D., Cannon, E. K. S., Liu, X., Gao, D., Clevenger, J., Dash, S., Ren, L., Moretzsohn, M. C., Shirasawa, K., Huang, W., Vidigal, B., Abernathy, B., Chu, Y., Niederhuth, C. E., Umale, P., . . . Ozias-Akins, P. (2016). The genome sequences of Arachis duranensis and Arachis ipaensis, the diploid ancestors of cultivated peanut. Nature Genetics, 48(4), 438-446.
Bhattacharjee, O., Raul, B., Ghosh, A., Bhardwaj, A., Bandyopadhyay, K., & Sinharoy, S. (2022). Nodule INception-independent epidermal events lead to bacterial entry during nodule development in peanut (Arachis hypogaea). New Phytologist, 236(6), 2265-2281.
Boisson-Dernier, A., Chabaud, M., Garcia, F., Bécard, G., Rosenberg, 889 C., & Barker, D. G. (2001). Agrobacterium rhizogenes-Transformed Roots of Medicago truncatula for the Study of Nitrogen-Fixing and Endomycorrhizal Symbiotic Associations. Molecular Plant-Microbe Interactions®, 14(6), 695-700.
Bray, N. L., Pimentel, H., Melsted, P., & Pachter, L. (2016). Near-optimal probabilistic RNA-seq quantification. Nature Biotechnology, 34(5), 525-527.
Clavijo, F., Diedhiou, I., Vaissayre, V., Brottier, L., Acolatse, J., Moukouanga, D., Crabos, A., Auguy, F., Franche, C., Gherbi, H., Champion, A., Hocher, V., Barker, D., Bogusz, D., Tisa, L. S., & Svistoonoff, S. (2015). The Casuarina NIN gene is transcriptionally activated throughout Frankia root infection as well as in response to bacterial diffusible signals. New Phytologist, 208(3), 887-903.
Crombez, H., Motte, H., & Beeckman, T. (2019). Tackling Plant Phosphate Starvation by the Roots. In Developmental Cell (Vol. 48, Issue 5, pp. 599-615). Cell Press.
Demina, I. V., Maity, P. J., Nagchowdhury, A., Ng, J. L. P., van der Graaff, E., Demchenko, K. N., Roitsch, T., Mathesius, U., & Pawlowski, K. (2019). Accumulation of and Response to Auxins in Roots and Nodules of the Actinorhizal Plant Datisca glomerata Compared to the Model Legume Medicago truncatula. Frontiers in Plant Science, 10.
Fan, H. M., Sun, C. H., Wen, L. Z., Liu, B. W., Ren, H., Sun, X., Ma, F. F., & Zheng, C. S. (2019). CmTCP20 Plays a Key Role in Nitrate and Auxin Signaling-Regulated Lateral Root Development in Chrysanthemum. Plant and Cell Physiology, 60(7), 1581-1594.
Freire-Rios, A., Tanaka, K., Crespo, I., van der Wijk, E., Sizentsova, Y., Levitsky, V., Lindhoud, S., Fontana, M., Hohlbein, J., Boer, D. R., Mironova, V., & Weijers, D. (2020). Architecture of DNA elements mediating ARF transcription factor binding and auxin responsive gene expression in Arabidopsis. Proceedings of the National Academy of Sciences, 117(39), 24557-24566.
Fu, M., Sun, J., Li, X., Guan, Y., & Xie, F. (2022). Asymmetric redundancy of soybean Nodule Inception (NIN) genes in root nodule symbiosis. Plant Physiology, 188(1), 477-489.
Gauthier-Coles, C., White, R. G., & Mathesius, U. (2019). Nodulating legumes are distinguished by a sensitivity to cytokinin in the root cortex leading to pseudonodule development. Frontiers in Plant Science, 9.
Gilbert, G. A., Knight, J. D., Vance, C. P., & Allan, D. L. (2000). Proteoid root development of phosphorus deficient lupin is mimicked by auxin and phosphonate. Annals of Botany, 85(6), 921-928.
Goodstein, D. M., Shu, S., Howson, R., Neupane, R., Hayes, R. D., Fazo, J., Mitros, T., Dirks, W., Hellsten, U., Putnam, N., & Rokhsar, D. S. (2012). Phytozome: A comparative platform for green plant genomics. Nucleic Acids Research, 40(D1).
Griesmann, M., Chang, Y., Liu, X., Song, Y., Haberer, G., Crook, M. B., Billault-Penneteau, B., Lauressergues, D., Keller, J., Imanishi, L., Roswanjaya, Y. P., Kohlen, W., Pujic, P., Battenberg, K., Alloisio, N., Liang, Y., Hilhorst, H., Salgado, M. G., Hocher, V., . . . Cheng, S. (2018). Phylogenomics reveals multiple losses of nitrogen-fixing root nodule symbiosis. Science, 361(6398).
Guan, P., Ripoll, J. J., Wang, R., Vuong, L., Bailey-Steinitz, L. J., Ye, D., & Crawford, N. M. (2017). Interacting TCP and NLP transcription factors control plant responses to nitrate availability. Proceedings of the National Academy of Sciences of the United States of America, 114(9), 2419-2424.
Hane, J. K., Ming, Y., Kamphuis, L. G., Nelson, M. N., Garg, G., Atkins, C. A., Bayer, P. E., Bravo, A., Bringans, S., Cannon, S., Edwards, D., Foley, R., Gao, L. L., Harrison, M. J., Huang, W., Hurgobin, B., Li, S., Liu, C. W., McGrath, A., . . . Singh, K. B. (2017). A comprehensive draft genome sequence for lupin (Lupinus angustifolius), an emerging health food: insights into plant-microbe interactions and legume evolution. Plant Biotechnology Journal, 15(3), 318-330.
Hiltenbrand, R., Thomas, J., McCarthy, H., Dykema, K. J., Spurr, A., Newhart, H., Winn, M. E., & Mukherjee, A. (2016). A developmental and molecular view of formation of auxin-induced nodule-like structures in land plants. Frontiers in Plant Science, 7(NOVEMBER2016).
Ibáñez, F., Wall, L., & Fabra, A. (2017). Starting points in plant-bacteria nitrogen-fixing symbioses: Intercellular invasion of the roots. In Journal of Experimental Botany (Vol. 68, Issue 8, pp. 1905-1918). Oxford University Press.
Imani, J., Li, L., Schäfer, P., & Kogel, K. H. (2011). STARTS—A stable root transformation system for rapid functional analyses of proteins of the monocot model plant barley. Plant Journal, 67(4), 726-735.
Irving, T. B., Chakraborty, S., Maia, L. G. S., Knaack, S., Conde, D., Schmidt, H. W., Triozzi, P. M., Simmons, C. H., Roy, S., Kirst, M., & Ané, J. M. (2022). An LCO-responsive homolog of NODULE INCEPTION positively regulates lateral root formation in Populus sp. Plant Physiology, 190(3), 1699-1714.
Journet, E. P., El-Gachtouli, N., Vernoud, V., de Billy, F., Pichon, M., Dedieu, A., Arnould, C., Morandi, D., Barker, D. G., & Gianinazzi-Pearson, V. (2001). Medicago truncatula ENOD11: A Novel RPRP-Encoding Early Nodulin Gene Expressed During Mycorrhization in Arbuscule-Containing Cells. Molecular Plant-Microbe Interactions®, 14(6), 737-748.
Kohlen, W., Ng, J. L. P., Deinum, E. E., & Mathesius, U. (2018a). Auxin transport, metabolism, and signaling during nodule initiation: Indeterminate and determinate nodules. In Journal of Experimental Botany (Vol. 69, Issue 2, pp. 229-244). Oxford University Press.
Kohlen, W., Ng, J. L. P., Deinum, E. E., & Mathesius, U. (2018b). Auxin transport, metabolism, and signaling during nodule initiation: Indeterminate and determinate nodules. In Journal of Experimental Botany (Vol. 69, Issue 2, pp. 229-244). Oxford University Press.
Konishi, M., & Yanagisawa, S. (2013). Arabidopsis NIN-like transcription factors have a central role in nitrate signaling. Nature Communications, 4.
Lerouge, P., Roche, P., Fauchert, C., Maillett, F., Truchett, G., Prome, J. C., & Denariet, J. (1990). Symbiotic host-specificity of Rhizobium me/i/oti is determined by a sulphated and acylated glucosamine oligosaccharide signal LETTERS TO NATURE. In NATURE (Vol. 344).
Li, X. R., Sun, J., Albinsky, D., Zarrabian, D., Hull, R., Lee, T., Jarratt-Barnham, E., Chiu, C. H., Jacobsen, A., Soumpourou, E., Albanese, A., Kohlen, W., Luginbuehl, L. H., Guillotin, B., Lawrensen, T., Lin, H., Murray, J., Wallington, E., Harwood, W., . . . Oldroyd, G. E. D. (2022). Nutrient regulation of lipochitooligosaccharide recognition in plants via NSP1 and NSP2. Nature Communications, 13(1).
Liao, Y., Smyth, G. K., & Shi, W. (2014). FeatureCounts: An efficient general purpose program for assigning sequence reads to genomic features. Bioinformatics, 30(7), 923-930.
Liu, J., & Bisseling, T. (2020). Evolution of nin and NIN-like genes in relation to nodule symbiosis. In Genes (Vol. 11, Issue 7, pp. 1-15). MDPI AG.
Liu, J., Rasing, M., Zeng, T., Klein, J., Kulikova, O., & Bisseling, T. (2021). NIN is essential for development of symbiosomes, suppression of defence and premature senescence in Medicago truncatula nodules. New Phytologist, 230(1), 290-303.
Liu, J., Rutten, L., Limpens, E., Van Der Molen, T., Van Velzen, R., Chen, R., Chen, Y., Geurts, R., Kohlen, W., Kulikova, O., & Bisseling, T. (2019). A remote cis-regulatory region is required for nin expression in the pericycle to initiate nodule primordium formation in Medicago truncatula. Plant Cell, 31(1), 68-83.
Liu, K. H., Niu, Y., Konishi, M., Wu, Y., Du, H., Sun Chung, H., Li, L., Boudsocq, M., McCormack, M., Maekawa, S., Ishida, T., Zhang, C., Shokat, K., Yanagisawa, S., & Sheen, J. (2017). Discovery of nitrate-CPK-NLP signaling in central nutrient-growth networks. Nature, 545(7654), 311-316.
Liu, K. H., Liu, M., Lin, Z., Wang, Z. F., Chen, B., Liu, C., Guo, A., Konishi, M., Yanagisawa, S., Wagner, G., & Sheen, J. (2022). NIN-like protein 7 transcription factor is a plant nitrate sensor. Science, 377(6613), 1419-1425.
Marchive, C., Roudier, F., Castaings, L., Bréhaut, V., Blondet, E., Colot, V., Meyer, C., & Krapp, A. (2013). Nuclear retention of the transcription factor NLP7 orchestrates the early response to nitrate in plants. Nature Communications, 4.
Marsh, J. F., Rakocevic, A., Mitra, R. M., Brocard, L., Sun, J., Eschstruth, A., Long, S. R., Schultze, M., Ratet, P., & Oldroyd, G. E. D. (2007). Medicago truncatula NIN is essential for rhizobial-independent nodule organogenesis induced by autoactive calcium/calmodulin-dependent protein kinase. Plant Physiology, 144(1), 324-335.
Mironova, V. V., Omelyanchuk, N. A., Wiebe, D. S., & Levitsky, V. G. (2014). Computational analysis of auxin responsive elements in the Arabidopsis thaliana L. genome. BMC Genomics, 15.
Mu, X., & Luo, J. (2019). Evolutionary analyses of NIN-like proteins in plants and their roles in nitrate signaling. In Cellular and Molecular Life Sciences (Vol. 76, Issue 19, pp. 3753-3764). Birkhauser Verlag AG.
Ng, J. L. P., Hassan, S., Truong, T. T., Hocart, C. H., Laffont, C., Frugier, F., & Mathesiusa, U. (2015). Flavonoids and auxin transport inhibitors rescue symbiotic nodulation in the Medicago truncatula cytokinin perception mutant crel. Plant Cell, 27(8), 2210-2226.
Nguyen, T. Van, Wibberg, D., Battenberg, K., Blom, J., Vanden Heuvel, B., Berry, A. M., Kalinowski, J., & Pawlowski, K. (2016). An assemblage of Frankia Cluster II strains from California contains the canonical nod genes and also the sulfotransferase gene nodH. BMC Genomics, 17(1).
Pawlowski, K., & Demchenko, K. N. (2012). The diversity of actinorhizal symbiosis. In Protoplasma (Vol. 249, Issue 4, pp. 967-979). Springer-Verlag Wien.
Persson, T., Battenberg, K., Demina, I. V., Vigil-Stenman, T., Heuvel, B. Vanden, Pujic, P., Facciotti, M. T., Wilbanks, E. G., O'Brien, A., Fournier, P., Hernandez, M. A. C., Herrera, A. M., Médigue, C., Normand, P., Pawlowski, K., & Berry, A. M. (2015). Candidatus Frankia datiscae Dg1, the Actinobacterial microsymbiont of Datisca glomerata, expresses the canonical nod genes NodABC in symbiosis with its host plant. PLoS ONE, 10(5).
Rightmyer, A. P., & Long, S. R. (2011). Pseudonodule Formation by Wild-Type and Symbiotic Mutant Medicago truncatula in Response to Auxin Transport Inhibitors. Molecular Plant-Microbe Interactions MPMI, 24(11), 1372-1384.
Roy, S., Robson, F., Lilley, J., Liu, C. W., Cheng, X., Wen, J., Walker, S., Sun, J., Cousins, D., Bone, C., Bennett, M. J., Downie, J. A., Swarup, R., Oldroyd, G., & Murray, J. D. (2017). MtLAX2, a functional homologue of the arabidopsis auxin influx transporter AUX1, is required for nodule organogenesis. Plant Physiology, 174(1), 326-338.
S. Andrews. (2010). FASTQC. A quality control tool for high throughput sequence data.
Sato, S., Nakamura, Y., Kaneko, T., Asamizu, E., Kato, T., Nakao, M., Sasamoto, S., Watanabe, A., Ono, A., Kawashima, K., Fujishiro, T., Katoh, M., Kohara, M., Kishida, Y., Minami, C., Nakayama, S., Nakazaki, N., Shimizu, Y., Shinpo, S., . . . Tabata, S. (2008). Genome structure of the legume, Lotus japonicus. DNA Research, 15(4), 227-239.
Schauser, L., Roussis, A., Stiller, J., & Stougaard, J. (1999). A plant regulator controlling development of symbiotic root nodules. Nature, 402(6758), 191-195.
Schiessl, K., Lilley, J. L. S., Lee, T., Tamvakis, I., Kohlen, W., Bailey, P. C., Thomas, A., Luptak, J., Ramakrishnan, K., Carpenter, M. D., Mysore, K. S., Wen, J., Ahnert, S., Grieneisen, V. A., & Oldroyd, G. E. D. (2019). NODULE INCEPTION Recruits the Lateral Root Developmental Program for Symbiotic Nodule Organogenesis in Medicago truncatula. Current Biology, 29(21), 3657-3668.e5.
Schmutz, J., Cannon, S. B., Schlueter, J., Ma, J., Mitros, T., Nelson, W., Hyten, D. L., Song, Q., Thelen, J. J., Cheng, J., Xu, D., Hellsten, U., May, G. D., Yu, Y., Sakurai, T., Umezawa, T., Bhattacharyya, M. K., Sandhu, D., Valliyodan, B., . . . Jackson, S. A. (2010). Genome sequence of the palaeopolyploid soybean. Nature, 463(7278), 178-183.
Shrestha, A., Zhong, S., Therrien, J., Huebert, T., Sato, S., Mun, T., Andersen, S. U., Stougaard, J., Lepage, A., Niebel, A., Ross, L., & Szczyglowski, K. (2021). Lotus japonicus Nuclear Factor YA1, a nodule emergence stage-specific regulator of auxin signaling. New Phytologist, 229(3), 1535-1552.
Skene, K. R. (2001). Cluster roots: model experimental tools for key biological problems. Journal of Experimental Botany, 52(suppl 1), 479-485.
Soyano, T., Hirakawa, H., Sato, S., Hayashi, M., & Kawaguchi, M. (2014). NODULE INCEPTION creates a long-distance negative feedback loop involved in homeostatic regulation of nodule organ production. Proceedings of the National Academy of Sciences of the United States of America, 111(40), 14607-14612.
Soyano, T., Kouchi, H., Hirota, A., & Hayashi, M. (2013). NODULE INCEPTION Directly Targets NF-Y Subunit Genes to Regulate Essential Processes of Root Nodule Development in Lotus japonicus. PLoS Genetics, 9(3).
Soyano, T., Shimoda, Y., Kawaguchi, M., & Hayashi, M. (2019). A shared gene drives lateral root development and root nodule symbiosis pathways in Lotus. Science, 366(6468), 1021-1023.
Suzuki, W., Konishi, M., & Yanagisawa, S. (2013). The evolutionary events necessary for the emergence of symbiotic nitrogen fixation in legumes may involve a loss of nitrate responsiveness of the NIN transcription factor. Plant Signaling and Behavior, 8(10).
Tang, H., Krishnakumar, V., Bidwell, S., Rosen, B., Chan, A., Zhou, S., Gentzbittel, L., Childs, K. L., Yandell, M., Gundlach, H., Mayer, K. F., Schwartz, D. C., & Town, C. D. (2014). An improved genome release (version Mt4.0) for the model legume Medicago truncatula. BMC Genomics, 15(1), 312.
Tuskan, G. A., DiFazio, S., Jansson, S., Bohlmann, J., Grigoriev, I., Hellsten, U., Putnam, N., Ralph, S., Rombauts, S., Salamov, A., Schein, J., Sterck, L., Aerts, A., Bhalerao, R. R., Bhalerao, R. P., Blaudez, D., Boerjan, W., Brun, A., Brunner, A., . . . Rokhsar, D. (2006). The Genome of Black Cottonwood, Populus trichocarpa (Torr. &amp; Gray). Science, 313(5793), 1596-1604.
Ulmasov, T., Hagen, G., & Guilfoyle, T. J. (1997). ARF1, a Transcription Factor That Binds to Auxin Response Elements. Science, 276(5320), 1865-1868.
van Velzen, R., Holmer, R., Bu, F., Rutten, L., van Zeijl, A., Liu, W., Santuari, L., Cao, Q., Sharma, T., Shen, D., Roswanjaya, Y., Wardhani, T. A. K., Kalhor, M. S., Jansen, J., van den Hoogen, J., Güngör, B., Hartog, M., Hontelez, J., Verver, J., . . . Geurts, R. (2018). Comparative genomics of the nonlegume Parasponia reveals insights into evolution of nitrogen-fixing rhizobium symbioses. Proceedings of the National Academy of Sciences, 115(20).
Varshney, R. K., Chen, W., Li, Y., Bharti, A. K., Saxena, R. K., Schlueter, J. A., Donoghue, M. T. A., Azam, S., Fan, G., Whaley, A. M., Farmer, A. D., Sheridan, J., Iwata, A., Tuteja, R., Penmetsa, R. V., Wu, W., Upadhyaya, H. D., Yang, S. P., Shah, T., . . . Jackson, S. A. (2012). Draft genome sequence of pigeonpea (Cajanus cajan), an orphan legume crop of resource-poor farmers. Nature 1074 Biotechnology, 30(1), 83-89.
Varshney, R. K., Song, C., Saxena, R. K., Azam, S., Yu, S., Sharpe, A. G., Cannon, S., Baek, J., Rosen, B. D., Tar'an, B., Millan, T., Zhang, X., Ramsay, L. D., Iwata, A., Wang, Y., Nelson, W., Farmer, A. D., Gaur, P. M., Soderlund, C., . . . Cook, D. R. (2013). Draft genome sequence of chickpea (Cicer arietinum) provides a resource for trait improvement. Nature Biotechnology, 31(3), 240-246.
Vernié, T., Kim, J., Frances, L., Ding, Y., Sun, J., Guan, D., Niebel, A., Gifford, M. L., de Carvalho-Niebel, F., & Oldroyd, G. E. D. (2015). The NIN transcription factor coordinates diverse nodulation programs in different tissues of the Medicago truncatula root. Plant Cell, 27(12), 3410-3424.
Vidal, E. A., Araus, V., Lu, C., Parry, G., Green, P. J., Coruzzi, G. M., & Gutiérrez, R. A. (2010). Nitrate-responsive miR393/AFB3 regulatory module controls root system architecture in Arabidopsis thaliana. Proceedings of the National Academy of Sciences of the United States of America, 107(9), 4477-4482.
Wang, X., Pearse, S. J., & Lambers, H. (2013). Cluster-root formation and carboxylate release in three Lupinus species as dependent on phosphorus supply, internal phosphorus concentration and relative growth rate. Annals of Botany, 112(7), 1449-1459.
Weber, E., Engler, C., Gruetzner, R., Werner, S., & Marillonnet, S. (2011). A modular cloning system for standardized assembly of multigene constructs. PLoS ONE, 6(2).
Wu, T. Y., Goh, H. Z., Azodi, C. B., Krishnamoorthi, S., Liu, M. J., & Urano, D. (2021). Evolutionarily conserved hierarchical gene regulatory networks for plant salt stress response. Nature Plants, 7(6), 787-799.
Xie, F., Murray, J. D., Kim, J., Heckmann, A. B., Edwards, A., Oldroyd, G. E. D., & Downie, J. A. (2012). Legume pectate lyase required for root infection by rhizobia. Proceedings of the National Academy of Sciences of the United States of America, 109(2), 633-638.
Zhang, H., & Forde, B. G. (1998). An Arabidopsis MADS Box Gene That Controls Nutrient-Induced Changes in Root Architecture. Science, 279(5349), 407-409.
Zhang, Y., Lee, C. W., Wehner, N., Imdahl, F., Svetlana, V., Weiste, C., Dröge-Laser, W., & Deeken, R. (2015). Regulation of Oncogene Expression in T-DNA-Transformed Host Plant Cells. PLoS Pathogens, 11(1), 1-27.

1. A genetically altered plant or part thereof comprising one or more genetic alterations that increase activity of a NODULE INCEPTION (NIN) protein or a NIN-like protein (NLP protein) in response to auxin signaling as compared to a control plant without the one or more genetic alterations.

2. The genetically altered plant or part thereof of claim 1, wherein the one or more genetic alterations comprise addition of one or more auxin response elements (AuxREs) operably linked to a nucleic acid encoding the NIN protein or the NLP protein.

3. The genetically altered plant or part thereof of claim 2, wherein the one or more genetic alterations comprise addition of two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more AuxREs.

4. The genetically altered plant or part thereof of claim 2, wherein the one or more AuxREs comprise a TGTCTC motif or a TGTCTN motif.

5. The genetically altered plant or part thereof of claim 2, wherein the one or more AuxREs are added between a cyclops binding site and a transcriptional start site of the nucleic acid encoding the NIN protein or the NLP protein.

6. The genetically altered plant or part thereof of claim 2, wherein the NIN protein or the NLP protein comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 649.

7. The genetically altered plant or part thereof of claim 2, wherein the nucleic acid encodes a NIN protein, and wherein the NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588.

8. (canceled)

9. The genetically altered plant or part thereof of claim 2, wherein the nucleic acid encodes a NIN/NLP1 orthogroup protein, and wherein the NIN/NLP1 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588.

10. The genetically altered plant or part thereof of claim 2, wherein the nucleic acid encodes a NLP2-3 orthogroup protein, and wherein the NLP2-3 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377.

11. The genetically altered plant or part thereof of claim 2, wherein the nucleic acid encodes a NLP4 orthogroup protein, and wherein the NLP4 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524.

12. The genetically altered plant or part thereof of claim 2, wherein the nucleic acid encodes a basal NIN/NLP orthogroup protein, and wherein the basal NIN/NLP orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550.

13. The genetically altered plant or part thereof of claim 2, wherein the one or more AuxREs are derived from a promoter of a nucleic acid encoding the NIN protein or the NLP protein in a legume or an actinorhizal plant species or wherein the one or more AuxREs are derived from a synthetic auxin responsive promoter, optionally wherein the synthetic auxin responsive promoter is pDR5.

14. The genetically altered plant or part thereof of claim 2, wherein the one or more AuxREs and/or the nucleic acid encoding the NIN protein or the NLP protein are heterologous or transfected.

15. The genetically altered plant or part thereof of claim 2, wherein the one or more AuxREs and/or the nucleic acid encoding the NIN protein or the NLP protein are endogenous.

16. (canceled)

17. (canceled)

18. The genetically altered plant or part thereof of claim 1, wherein the genetically altered plant is a monocot, and wherein the genetically altered plant is selected from the group consisting of corn, rice, wheat, barley, sorghum, millet, oat, and rye.

19. The genetically altered plant or part thereof of claim 1, wherein the genetically altered plant is selected from the group consisting of apple, pear, plum, apricot, peach, almond, walnut, cherry, strawberry, raspberry, blackberry, red currant, black currant, melon, cucumber, pumpkin, squash, grape, hemp, hops, birch, beech, jujube, cassava, poplar, chestnut, citrus, potato, tomato, sweet potato, Datisca spp., Trema spp., and Jatropha spp.

20. The genetically altered plant or part thereof of claim 1, wherein the genetically altered plant is a non-nodulating plant and the increased activity of the NODULE INCEPTION (NIN) protein or the NIN-like protein (NLP protein) in response to auxin signaling induces formation of actinorhizal nodule-like structures.

21. A method of producing the genetically altered plant or part thereof of claim 1, comprising introducing a genetic alteration to the plant comprising a first nucleic acid sequence comprising one or more AuxREs.

22. The method of claim 21, further comprising introducing a second nucleic acid sequence encoding the NIN protein or the NLP protein, wherein the first nucleic acid sequence and the second nucleic acid sequence are operably linked.

23. The method of claim 21, wherein the first nucleic acid sequence is inserted into the genome of the plant so that the nucleic acid sequence is operably linked to an endogenous nucleic acid sequence encoding the NIN protein or the NLP protein.

24. The method of claim 21, wherein the one or more AuxREs comprise a TGTCTC motif or a TGTCTN motif.

25. The method of claim 21, wherein the first nucleic acid sequence comprises a synthetic auxin responsive promoter, optionally wherein the synthetic auxin responsive promoter is pDR5.

26. The method of claim 22, wherein the NIN protein or the NLP protein comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 649.

27. The method of claim 22,
(i) wherein the NIN protein or NLP protein is a NIN protein, wherein the NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588;
(ii) wherein the NIN protein or the NLP protein is a NIN/NLP1 orthogroup protein, wherein the NIN/NLP1 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588;
(iii) wherein the NIN protein or the NLP protein is a NLP2-3 orthogroup protein, wherein the NLP2-3 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377;
(iv) wherein the NIN protein or the NLP protein is a NLP4 orthogroup protein, wherein the NLP4 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524; or
(v) wherein the NIN protein or the NLP protein is a basal NIN/NLP orthogroup protein, wherein the basal NIN/NLP orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550.

28. A method of producing the genetically altered plant or part thereof of claim 1, comprising genetically modifying the plant or part thereof by transforming the plant or part thereof with one or more gene editing components that target an endogenous nuclear genome sequence between a cyclops binding site and a transcriptional start site of the nucleic acid encoding the NIN protein or the NLP protein and introduce one or more AuxREs.

29. The method of claim 28, wherein the one or more gene editing components comprise:
(a) a ribonucleoprotein complex that targets the nuclear genome sequence; a vector comprising a TALEN protein encoding sequence, wherein the TALEN protein targets the nuclear genome sequence;
(b) a vector comprising a ZFN protein encoding sequence, wherein the ZFN protein targets the nuclear genome sequence;
(c) an oligonucleotide donor (OND), wherein the OND targets the nuclear genome sequence; or
(d) a vector CRISPR/Cas enzyme encoding sequence and a targeting sequence, wherein the targeting sequence targets the nuclear genome sequence.

30. The method of claim 28, wherein the one or more AuxREs comprise a TGTCTC motif or a TGTCTN motif.

31. The method of claim 28, wherein the NIN protein or the NLP protein comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 649.

32. The method of claim 28,
(i) wherein the NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588;
(ii) wherein the NIN protein or the NLP protein is a NIN/NLP1 orthogroup protein, wherein the NIN/NLP1 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588;
(iii) wherein the NIN protein or the NLP protein is a NLP2-3 orthogroup protein, wherein the NLP2-3 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377;
(iv) wherein the NIN protein or the NLP protein is a NLP4 orthogroup protein, wherein the NLP4 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524; or
(v) wherein the NIN protein or the NLP protein is a basal NIN/NLP orthogroup protein, wherein the basal NIN/NLP orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550.

33. An expression vector, isolated DNA molecule, or recombinant nucleic acid comprising a nucleic acid sequence comprising one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more AuxREs from an endogenous plant promoter, or a synthetic promoter comprising one or more, two or more, three or more, four or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more AuxREs.

34. The expression vector, isolated DNA molecule, or recombinant nucleic acid of claim 33, wherein the one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, or twenty-four or more AuxREs are operably linked to a second nucleic acid sequence encoding a NIN protein or a NLP protein.

35. The expression vector, isolated DNA molecule, or recombinant nucleic acid of claim 33, wherein the one or more AuxREs comprise a TGTCTC motif or a TGTCTN motif.

36. The expression vector, isolated DNA molecule, or recombinant nucleic acid of claim 34, wherein the NIN protein or the NLP protein comprises an amino acid sequence with at least 80%, at least 85%, at least 90%, at least 91%, at least 92%, at least 93%, at least 94%, at least 95%, at least 96%, at least 97%, at least 98%, at least 99%, or 100% identity to SEQ ID NO: 649.

37. The expression vector, isolated DNA molecule, or recombinant nucleic acid of claim 34,
(i) wherein the NIN protein or NLP protein is a NIN protein, wherein the NIN protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588;
(ii) wherein the NIN protein or NLP protein is a NIN/NLP1 orthogroup protein, wherein the NIN/NLP1 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22; SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 108, SEQ ID NO: 109, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 177, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 196, SEQ ID NO: 197, SEQ ID NO: 198, SEQ ID NO: 199, SEQ ID NO: 200, SEQ ID NO: 201, SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216, SEQ ID NO: 217, SEQ ID NO: 218, SEQ ID NO: 219, SEQ ID NO: 220, SEQ ID NO: 221, SEQ ID NO: 222, SEQ ID NO: 223, SEQ ID NO: 224, SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, SEQ ID NO: 234, SEQ ID NO: 235, SEQ ID NO: 236, SEQ ID NO: 551, SEQ ID NO: 552, SEQ ID NO: 553, SEQ ID NO: 554, SEQ ID NO: 555, SEQ ID NO: 556, SEQ ID NO: 557, SEQ ID NO: 558, SEQ ID NO: 559, SEQ ID NO: 560, SEQ ID NO: 561, SEQ ID NO: 562, SEQ ID NO: 563, SEQ ID NO: 564, SEQ ID NO: 565, SEQ ID NO: 566, SEQ ID NO: 567, SEQ ID NO: 568, SEQ ID NO: 569, SEQ ID NO: 570, SEQ ID NO: 571, SEQ ID NO: 572, SEQ ID NO: 573, SEQ ID NO: 574, SEQ ID NO: 575, SEQ ID NO: 576, SEQ ID NO: 577, SEQ ID NO: 578, SEQ ID NO: 579, SEQ ID NO: 580, SEQ ID NO: 581, SEQ ID NO: 582, SEQ ID NO: 583, SEQ ID NO: 584, SEQ ID NO: 585, SEQ ID NO: 586, SEQ ID NO: 587, and SEQ ID NO: 588;
(iii) wherein the NIN protein or NLP protein is a NLP2-3 orthogroup protein, wherein the NLP2-3 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 237, SEQ ID NO: 238, SEQ ID NO: 239, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 250, SEQ ID NO: 251, SEQ ID NO: 252, SEQ ID NO: 253, SEQ ID NO: 254, SEQ ID NO: 255, SEQ ID NO: 256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265, SEQ ID NO: 266, SEQ ID NO: 267, SEQ ID NO: 268, SEQ ID NO: 269, SEQ ID NO: 270, SEQ ID NO: 271, SEQ ID NO: 273, SEQ ID NO: 274, SEQ ID NO: 275, SEQ ID NO: 276, SEQ ID NO: 277, SEQ ID NO: 278, SEQ ID NO: 279, SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, SEQ ID NO: 289, SEQ ID NO: 290, SEQ ID NO: 291, SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 371, SEQ ID NO: 372, SEQ ID NO: 373, SEQ ID NO: 374, SEQ ID NO: 375, SEQ ID NO: 376, and SEQ ID NO: 377;
(iv) wherein the NIN protein or NLP protein is an NLP4 orthogroup protein, wherein the NLP4 orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, SEQ ID NO: 408, SEQ ID NO: 409, SEQ ID NO: 410, SEQ ID NO: 411, SEQ ID NO: 412, SEQ ID NO: 413, SEQ ID NO: 414, SEQ ID NO: 415, SEQ ID NO: 417, SEQ ID NO: 418, SEQ ID NO: 419, SEQ ID NO: 420, SEQ ID NO: 421, SEQ ID NO: 422, SEQ ID NO: 423, SEQ ID NO: 424, SEQ ID NO: 425, SEQ ID NO: 426, SEQ ID NO: 427, SEQ ID NO: 428, SEQ ID NO: 429, SEQ ID NO: 430, SEQ ID NO: 431, SEQ ID NO: 432, SEQ ID NO: 433, SEQ ID NO: 434, SEQ ID NO: 435, SEQ ID NO: 436, SEQ ID NO: 437, SEQ ID NO: 438, SEQ ID NO: 439, SEQ ID NO: 440, SEQ ID NO: 441, SEQ ID NO: 442, SEQ ID NO: 443, SEQ ID NO: 444, SEQ ID NO: 445, SEQ ID NO: 446, SEQ ID NO: 447, SEQ ID NO: 448, SEQ ID NO: 449, SEQ ID NO: 450, SEQ ID NO: 451, SEQ ID NO: 452, SEQ ID NO: 453, SEQ ID NO: 455, SEQ ID NO: 456, SEQ ID NO: 457, SEQ ID NO: 458, SEQ ID NO: 459, SEQ ID NO: 460, SEQ ID NO: 461, SEQ ID NO: 462, SEQ ID NO: 463, SEQ ID NO: 464, SEQ ID NO: 465, SEQ ID NO: 466, SEQ ID NO: 467, SEQ ID NO: 468, SEQ ID NO: 469, SEQ ID NO: 470, SEQ ID NO: 471, SEQ ID NO: 472, SEQ ID NO: 473, SEQ ID NO: 474, SEQ ID NO: 475, SEQ ID NO: 476, SEQ ID NO: 477, SEQ ID NO: 478, SEQ ID NO: 479, SEQ ID NO: 480, SEQ ID NO: 481, SEQ ID NO: 482, SEQ ID NO: 483, SEQ ID NO: 484, SEQ ID NO: 485, SEQ ID NO: 486, SEQ ID NO: 487, SEQ ID NO: 488, SEQ ID NO: 489, SEQ ID NO: 490, SEQ ID NO: 491, SEQ ID NO: 492, SEQ ID NO: 493, SEQ ID NO: 494, SEQ ID NO: 495, SEQ ID NO: 496, SEQ ID NO: 497, SEQ ID NO: 498, SEQ ID NO: 499, SEQ ID NO: 500, SEQ ID NO: 501, SEQ ID NO: 502, SEQ ID NO: 504, SEQ ID NO: 505, SEQ ID NO: 506, SEQ ID NO: 507, SEQ ID NO: 508, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, and SEQ ID NO: 524; or
(v) wherein the NIN protein or NLP protein is a basal NIN/NLP orthogroup protein, wherein the basal NIN/NLP orthogroup protein has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to an amino acid sequence selected from the group consisting of SEQ ID NO: 525, SEQ ID NO: 526, SEQ ID NO: 527, SEQ ID NO: 528, SEQ ID NO: 529, SEQ ID NO: 530, SEQ ID NO: 531, SEQ ID NO: 532, SEQ ID NO: 533, SEQ ID NO: 534, SEQ ID NO: 535, SEQ ID NO: 536, SEQ ID NO: 537, SEQ ID NO: 538, SEQ ID NO: 539, SEQ ID NO: 540, SEQ ID NO: 541, SEQ ID NO: 542, SEQ ID NO: 543, SEQ ID NO: 544, SEQ ID NO: 545, SEQ ID NO: 546, SEQ ID NO: 547, SEQ ID NO: 548, SEQ ID NO: 549, and SEQ ID NO: 550.

38. A bacterial cell or an Agrobacterium cell comprising the expression vector, isolated DNA molecule, or recombinant nucleic acid of claim 33.

39. A genetically modified plant, plant part, plant cell, or seed comprising the expression vector, isolated DNA molecule, or recombinant nucleic acid of claim 33.

40. A composition or kit comprising the expression vector, isolated DNA molecule, or recombinant nucleic acid of claim 33.

41. (canceled)

42. A method of inducing symbiotic organogenesis, inducing symbiotic organogenesis in the absence of rhizobial bacteria recognized by the plant, and/or inducing infection of a symbiotic organ comprising introducing a genetic alteration via the expression vector, isolated DNA molecule, or recombinant nucleic acid of claim 33.

43. (canceled)

44. (canceled)
